SEQUENCE LISTING (96 sequences)

SEQ ID NO: 1 - protein, 11 aa, synthetic construct
  REGION 1..11: note = synthetic polypeptide
VSGWRLFKKIS

SEQ ID NO: 2 - protein, 11 aa, synthetic construct
  REGION 1..11: note = synthetic polypeptide
GSMLFRVTINS

SEQ ID NO: 3 - protein, 148 aa, synthetic construct
  REGION 1..148: note = synthetic polypeptide
MVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPD

SEQ ID NO: 4 - protein, 18 aa, synthetic construct
  REGION 1..18: note = synthetic polypeptide
GGGSGHHHHHHHHGGGSG

SEQ ID NO: 5 - DNA, 1549 bp, synthetic construct
  misc_feature 1..1549: note = beta-9-L73J (light chain) nucleotide sequence
  source 1..1549: mol_type = other DNA
atgggtagtatgctgtttcgtgttaccattaacagcggtggtggtagcggtcatcatcaccaccatcaccatcatggtggtggcagtggtatggcaagcgatattcagatgacccagagcccgagcagcctgagcgcaagcgttggtgatcgtgttaccattacctgtcgtgcaagccagagcgttagcagcgcggttgcatggtatcagcagaaaccgggtaaagcaccgaaactgctgatttatagcgcaagcagcctgtatagcggtgttccgagccgttttagcggtagccgtagcggcaccgattttaccctgaccattagcagcctgcagccggaagattttgcaacctattattgtcagcagggctattattacccgtttacctttggtcagggcaccaaagttgaaattaaacgtaccgttgcagcaccgagcgtttttatctttccgcctagcgatagccagctgaaaagcggtacagccagcgttgtttgtctgctgaataacttttatccgcgtgaagcaaaagttcagtggaaagttgataatgcactgcagagcggtaatagccaagaaagcgttaccgaacaggatagcaaagatagcacctatagcctgagcagcaccctgaccctgagcaaagcagattatgaaaaacacaaagtgtatgcctgcgaagttacccatcagggtctgagcagtccggttaccaaaagctttaatcgtggtgaatgttaattaactcgaggctgagcaaagcagactactaataacataaagtctacgccggacgcatcgtggccctagtacgcaagttcacgtaaaaagggtaactagaggttgaggtgattttatggaaattagcgaagttcagctggttgaaagcggtggtggtctggttcagcctggtggtagcctgcgtctgagctgtgcagcaagcggttttaatctgtatagcagctatattcattgggttcgtcaggcaccgggtaaaggtctggaatgggttgcaagcatttatccgtatagctcatataccagctatgccgatagcgttaaaggtcgttttaccattagcgcagataccagcaaaaataccgcatacctgcagatgaatagtctgcgtgcagaggataccgcagtgtattattgtgcacgttattatggtttcgccatggattattggggtcagggcaccctggttaccgttagcagcgcaagcaccaaaggtccgagcgtttttccgctggcaccgagcagcaaaagcaccagcggtggcaccgcagcactgggttgtctggttaaagattattttccggaaccggttaccgtgagctggaatagcggtgcactgaccagtggtgttcatacctttccggcagttctgcagagcagcggtctgtatagtctgagcagcgttgttaccgttccgagcagtagcctgggcacccagacctatatttgtaatgttaatcataaaccgagcaacaccaaagtggataaaaaagttgaaccgaaaagctgcgataaaacccatacctaa

SEQ ID NO: 6 - protein, 247 aa, synthetic construct
  REGION 1..247: note = Beta9-His Tag-light chain
MGSMLFRVTINSGGGSGHHHHHHHHGGGSGMASDIQMTQSPSSLSASVGDRVTITCRASQSVSSAVAWYQQKPGKAPKLLIYSASSLYSGVPSRFSGSRSGTDFTLTISSLQPEDFATYYCQQGYYYPFTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 229 aa, synthetic construct
  REGION 1..229: note = heavy chain
MEISEVQLVESGGGLVQPGGSLRLSCAASGFNLYSSYIHWVRQAPGKGLEWVASIYPYSSYTSYADSVKGRFTISADTSKNTAYLQMNSLRAEDTAVYYCARYYGFAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 8 - DNA, 1549 bp, synthetic construct
  misc_feature 1..1549: note = Beta10*-L73J
  source 1..1549: mol_type = other DNA
atggtgagcggttggcgtctgttcaaaaaaatcagcggtggtggtagcggtcatcatcaccaccatcaccatcatggtggtggcagtggtatggcaagcgatattcagatgacccagagcccgagcagcctgagcgcaagcgttggtgatcgtgttaccattacctgtcgtgcaagccagagcgttagcagcgcggttgcatggtatcagcagaaaccgggtaaagcaccgaaactgctgatttatagcgcaagcagcctgtatagcggtgttccgagccgttttagcggtagccgtagcggcaccgattttaccctgaccattagcagcctgcagccggaagattttgcaacctattattgtcagcagggctattattacccgtttacctttggtcagggcaccaaagttgaaattaaacgtaccgttgcagcaccgagcgtttttatctttccgcctagcgatagccagctgaaaagcggtacagccagcgttgtttgtctgctgaataacttttatccgcgtgaagcaaaagttcagtggaaagttgataatgcactgcagagcggtaatagccaagaaagcgttaccgaacaggatagcaaagatagcacctatagcctgagcagcaccctgaccctgagcaaagcagattatgaaaaacacaaagtgtatgcctgcgaagttacccatcagggtctgagcagtccggttaccaaaagctttaatcgtggtgaatgttaattaactcgaggctgagcaaagcagactactaataacataaagtctacgccggacgcatcgtggccctagtacgcaagttcacgtaaaaagggtaactagaggttgaggtgattttatggaaattagcgaagttcagctggttgaaagcggtggtggtctggttcagcctggtggtagcctgcgtctgagctgtgcagcaagcggttttaatctgtatagcagctatattcattgggttcgtcaggcaccgggtaaaggtctggaatgggttgcaagcatttatccgtatagctcatataccagctatgccgatagcgttaaaggtcgttttaccattagcgcagataccagcaaaaataccgcatacctgcagatgaatagtctgcgtgcagaggataccgcagtgtattattgtgcacgttattatggtttcgccatggattattggggtcagggcaccctggttaccgttagcagcgcaagcaccaaaggtccgagcgtttttccgctggcaccgagcagcaaaagcaccagcggtggcaccgcagcactgggttgtctggttaaagattattttccggaaccggttaccgtgagctggaatagcggtgcactgaccagtggtgttcatacctttccggcagttctgcagagcagcggtctgtatagtctgagcagcgttgttaccgttccgagcagtagcctgggcacccagacctatatttgtaatgttaatcataaaccgagcaacaccaaagtggataaaaaagttgaaccgaaaagctgcgataaaacccatacctaa

SEQ ID NO: 9 - protein, 247 aa, synthetic construct
  REGION 1..247: note = Beta10*-His Tag-light chain
MVSGWRLFKKISGGGSGHHHHHHHHGGGSGMASDIQMTQSPSSLSASVGDRVTITCRASQSVSSAVAWYQQKPGKAPKLLIYSASSLYSGVPSRFSGSRSGTDFTLTISSLQPEDFATYYCQQGYYYPFTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 229 aa, synthetic construct; identical to SEQ ID NO: 7
  REGION 1..229: note = heavy chain

SEQ ID NO: 11 - DNA, 1546 bp, synthetic construct
  misc_feature 1..1546: note = 73JH-Beta9
  source 1..1546: mol_type = other DNA
atggcaagcgatattcagatgacccagagcccgagcagcctgagcgcaagcgttggtgatcgtgttaccattacctgtcgtgcaagccagagcgttagcagcgcggttgcatggtatcagcagaaaccgggtaaagcaccgaaactgctgatttatagcgcaagcagcctgtatagcggtgttccgagccgttttagcggtagccgtagcggcaccgattttaccctgaccattagcagcctgcagccggaagattttgcaacctattattgtcagcagggctattattacccgtttacctttggtcagggcaccaaagttgaaattaaacgtaccgttgcagcaccgagcgtttttatctttccgcctagcgatagccagctgaaaagcggtacagccagcgttgtttgtctgctgaataacttttatccgcgtgaagcaaaagttcagtggaaagttgataatgcactgcagagcggtaatagccaagaaagcgttaccgaacaggatagcaaagatagcacctatagcctgagcagcaccctgaccctgagcaaagcagattatgaaaaacacaaagtgtatgcctgcgaagttacccatcagggtctgagcagtccggttaccaaaagctttaatcgtggtgaatgttaattaactcgaggctgagcaaagcagactactaataacataaagtctacgccggacgcatcgtggccctagtacgcaagttcacgtaaaaagggtaactagaggttgaggtgattttatggaaattagcgaagttcagctggttgaaagcggtggtggtctggttcagcctggtggtagcctgcgtctgagctgtgcagcaagcggttttaatctgtatagcagctatattcattgggttcgtcaggcaccgggtaaaggtctggaatgggttgcaagcatttatccgtatagctcatataccagctatgccgatagcgttaaaggtcgttttaccattagcgcagataccagcaaaaataccgcatacctgcagatgaatagtctgcgtgcagaggataccgcagtgtattattgtgcacgttattatggtttcgccatggattattggggtcagggcaccctggttaccgttagcagcgcaagcaccaaaggtccgagcgtttttccgctggcaccgagcagcaaaagcaccagcggtggcaccgcagcactgggttgtctggttaaagattattttccggaaccggttaccgtgagctggaatagcggtgcactgaccagtggtgttcatacctttccggcagttctgcagagcagcggtctgtatagtctgagcagcgttgttaccgttccgagcagtagcctgggcacccagacctatatttgtaatgttaatcataaaccgagcaacaccaaagtggataaaaaagttgaaccgaaaagctgcgataaaacccataccggtggtggtagcggtcatcatcaccaccatcaccatcatggtggtggcagtggtggtagtatgctgtttcgtgttaccattaacagctaa

SEQ ID NO: 12 - protein, 217 aa, synthetic construct
  REGION 1..217: note = light chain
MASDIQMTQSPSSLSASVGDRVTITCRASQSVSSAVAWYQQKPGKAPKLLIYSASSLYSGVPSRFSGSRSGTDFTLTISSLQPEDFATYYCQQGYYYPFTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 258 aa, synthetic construct
  REGION 1..258: note = heavy chain-His Tag-Beta9
MEISEVQLVESGGGLVQPGGSLRLSCAASGFNLYSSYIHWVRQAPGKGLEWVASIYPYSSYTSYADSVKGRFTISADTSKNTAYLQMNSLRAEDTAVYYCARYYGFAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGSGHHHHHHHHGGGSGGSMLFRVTINS

SEQ ID NO: 14 - DNA, 1546 bp, synthetic construct
  misc_feature 1..1546: note = 73JH-Beta10*
  source 1..1546: mol_type = other DNA
atggcaagcgatattcagatgacccagagcccgagcagcctgagcgcaagcgttggtgatcgtgttaccattacctgtcgtgcaagccagagcgttagcagcgcggttgcatggtatcagcagaaaccgggtaaagcaccgaaactgctgatttatagcgcaagcagcctgtatagcggtgttccgagccgttttagcggtagccgtagcggcaccgattttaccctgaccattagcagcctgcagccggaagattttgcaacctattattgtcagcagggctattattacccgtttacctttggtcagggcaccaaagttgaaattaaacgtaccgttgcagcaccgagcgtttttatctttccgcctagcgatagccagctgaaaagcggtacagccagcgttgtttgtctgctgaataacttttatccgcgtgaagcaaaagttcagtggaaagttgataatgcactgcagagcggtaatagccaagaaagcgttaccgaacaggatagcaaagatagcacctatagcctgagcagcaccctgaccctgagcaaagcagattatgaaaaacacaaagtgtatgcctgcgaagttacccatcagggtctgagcagtccggttaccaaaagctttaatcgtggtgaatgttaattaactcgaggctgagcaaagcagactactaataacataaagtctacgccggacgcatcgtggccctagtacgcaagttcacgtaaaaagggtaactagaggttgaggtgattttatggaaattagcgaagttcagctggttgaaagcggtggtggtctggttcagcctggtggtagcctgcgtctgagctgtgcagcaagcggttttaatctgtatagcagctatattcattgggttcgtcaggcaccgggtaaaggtctggaatgggttgcaagcatttatccgtatagctcatataccagctatgccgatagcgttaaaggtcgttttaccattagcgcagataccagcaaaaataccgcatacctgcagatgaatagtctgcgtgcagaggataccgcagtgtattattgtgcacgttattatggtttcgccatggattattggggtcagggcaccctggttaccgttagcagcgcaagcaccaaaggtccgagcgtttttccgctggcaccgagcagcaaaagcaccagcggtggcaccgcagcactgggttgtctggttaaagattattttccggaaccggttaccgtgagctggaatagcggtgcactgaccagtggtgttcatacctttccggcagttctgcagagcagcggtctgtatagtctgagcagcgttgttaccgttccgagcagtagcctgggcacccagacctatatttgtaatgttaatcataaaccgagcaacaccaaagtggataaaaaagttgaaccgaaaagctgcgataaaacccataccggtggtggtagcggtcatcatcaccaccatcaccatcatggtggtggcagtggtgtgagcggttggcgtctgttcaaaaaaatcagctaa

SEQ ID NO: 15 - protein, 217 aa, synthetic construct; identical to SEQ ID NO: 12
  REGION 1..217: note = light chain

SEQ ID NO: 16 - protein, 258 aa, synthetic construct
  REGION 1..258: note = heavy chain-HisTag-Beta9
MEISEVQLVESGGGLVQPGGSLRLSCAASGFNLYSSYIHWVRQAPGKGLEWVASIYPYSSYTSYADSVKGRFTISADTSKNTAYLQMNSLRAEDTAVYYCARYYGFAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGSGHHHHHHHHGGGSGVSGWRLFKKIS

SEQ ID NO: 17 - DNA, 1555 bp, synthetic construct
  misc_feature 1..1555: note = Beta9-HTras
  source 1..1555: mol_type = other DNA
atggcaagcgatattcagatgacccagagcccgagcagcctgagcgcaagcgttggtgatcgtgttaccattacctgtcgtgcaagccaggatgttaataccgcagttgcatggtatcagcagaaaccgggtaaagcaccgaaactgctgatttatagcgcaagctttctgtatagcggtgttccgagccgttttagcggtagccgtagcggcaccgattttaccctgaccattagcagcctgcagccggaagattttgcaacctattattgtcagcagcattacaccacccctccgacctttggtcagggcaccaaagttgaaattaaacgtaccgttgcagcaccgagcgtttttatctttccgcctagcgatgagcagctgaaaagcggtacagccagcgttgtttgtctgctgaataacttttatccgcgtgaagcaaaagttcagtggaaagttgataatgcactgcagagcggtaatagccaagaaagcgttaccgaacaggatagcaaagatagcacctatagcctgagcagcaccctgaccctgagcaaagcagattatgaaaaacacaaagtgtatgcctgcgaagttacccatcagggtctgagcagtccggttaccaaaagctttaatcgtggtgaatgttaattaactcgaggctgagcaaagcagactactaataacataaagtctacgccggacgcatcgtggccctagtacgcaagttcacgtaaaaagggtaactagaggttgaggtgattttatgggtagtatgctgtttcgtgttaccattaacagcggtggtggtagcggtcatcatcaccaccatcaccatcatggtggtggcagtggtgaaattagcgaagttcagctggttgaaagcggtggtggtctggttcagcctggtggtagcctgcgtctgagctgtgcagcaagcggttttaacattaaagatacctatatccactgggttcgtcaggcaccgggtaaaggtctggaatgggttgcacgtatttatccgaccaatggttatacccgttatgccgatagcgttaaaggtcgttttaccattagcgcagataccagcaaaaataccgcatacctgcagatgaatagtctgcgtgcagaggataccgcagtgtattattgtagccgttggggtggtgatggtttttatgcaatggattattggggtcagggcaccctggttaccgttagcagcgcaagcaccaaaggtccgagcgtttttccgctggcaccgagcagcaaaagcaccagcggtggcaccgcagcactgggttgtctggttaaagattattttccggaaccggttaccgtgagctggaatagcggtgcactgaccagtggtgttcatacctttccggcagttctgcagagcagcggtctgtatagtctgagcagcgttgttaccgttccgagcagtagcctgggcacccagacctatatttgtaatgttaatcataaaccgagcaacaccaaagtggataaaaaagttgaaccgaaaagctgcgataaaacccatacctaa

SEQ ID NO: 18 - protein, 217 aa, synthetic construct
  REGION 1..217: note = light chain
MASDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 261 aa, synthetic construct
  REGION 1..261: note = Beta9-His Tag-heavy chain
MGSMLFRVTINSGGGSGHHHHHHHHGGGSGEISEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 20 - DNA, 1555 bp, synthetic construct
  misc_feature 1..1555: note = Beta10*-HTras
  source 1..1555: mol_type = other DNA
atggcaagcgatattcagatgacccagagcccgagcagcctgagcgcaagcgttggtgatcgtgttaccattacctgtcgtgcaagccaggatgttaataccgcagttgcatggtatcagcagaaaccgggtaaagcaccgaaactgctgatttatagcgcaagctttctgtatagcggtgttccgagccgttttagcggtagccgtagcggcaccgattttaccctgaccattagcagcctgcagccggaagattttgcaacctattattgtcagcagcattacaccacccctccgacctttggtcagggcaccaaagttgaaattaaacgtaccgttgcagcaccgagcgtttttatctttccgcctagcgatgagcagctgaaaagcggtacagccagcgttgtttgtctgctgaataacttttatccgcgtgaagcaaaagttcagtggaaagttgataatgcactgcagagcggtaatagccaagaaagcgttaccgaacaggatagcaaagatagcacctatagcctgagcagcaccctgaccctgagcaaagcagattatgaaaaacacaaagtgtatgcctgcgaagttacccatcagggtctgagcagtccggttaccaaaagctttaatcgtggtgaatgttaattaactcgaggctgagcaaagcagactactaataacataaagtctacgccggacgcatcgtggccctagtacgcaagttcacgtaaaaagggtaactagaggttgaggtgattttatggtgagcggttggcgtctgttcaaaaaaatcagcggtggtggtagcggtcatcatcaccaccatcaccatcatggtggtggcagtggtgaaattagcgaagttcagctggttgaaagcggtggtggtctggttcagcctggtggtagcctgcgtctgagctgtgcagcaagcggttttaacattaaagatacctatatccactgggttcgtcaggcaccgggtaaaggtctggaatgggttgcacgtatttatccgaccaatggttatacccgttatgccgatagcgttaaaggtcgttttaccattagcgcagataccagcaaaaataccgcatacctgcagatgaatagtctgcgtgcagaggataccgcagtgtattattgtagccgttggggtggtgatggtttttatgcaatggattattggggtcagggcaccctggttaccgttagcagcgcaagcaccaaaggtccgagcgtttttccgctggcaccgagcagcaaaagcaccagcggtggcaccgcagcactgggttgtctggttaaagattattttccggaaccggttaccgtgagctggaatagcggtgcactgaccagtggtgttcatacctttccggcagttctgcagagcagcggtctgtatagtctgagcagcgttgttaccgttccgagcagtagcctgggcacccagacctatatttgtaatgttaatcataaaccgagcaacaccaaagtggataaaaaagttgaaccgaaaagctgcgataaaacccatacctaa

SEQ ID NO: 21 - protein, 217 aa, synthetic construct; identical to SEQ ID NO: 18
  REGION 1..217: note = light chain

SEQ ID NO: 22 - protein, 261 aa, synthetic construct
  REGION 1..261: note = Beta10*-His Tag-heavy chain
MVSGWRLFKKISGGGSGHHHHHHHHGGGSGEISEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 23 - DNA, 1555 bp, synthetic construct
  misc_feature 1..1555: note = TrasH-Beta9
  source 1..1555: mol_type = other DNA
atggcaagcgatattcagatgacccagagcccgagcagcctgagcgcaagcgttggtgatcgtgttaccattacctgtcgtgcaagccaggatgttaataccgcagttgcatggtatcagcagaaaccgggtaaagcaccgaaactgctgatttatagcgcaagctttctgtatagcggtgttccgagccgttttagcggtagccgtagcggcaccgattttaccctgaccattagcagcctgcagccggaagattttgcaacctattattgtcagcagcattacaccacccctccgacctttggtcagggcaccaaagttgaaattaaacgtaccgttgcagcaccgagcgtttttatctttccgcctagcgatgagcagctgaaaagcggtacagccagcgttgtttgtctgctgaataacttttatccgcgtgaagcaaaagttcagtggaaagttgataatgcactgcagagcggtaatagccaagaaagcgttaccgaacaggatagcaaagatagcacctatagcctgagcagcaccctgaccctgagcaaagcagattatgaaaaacacaaagtgtatgcctgcgaagttacccatcagggtctgagcagtccggttaccaaaagctttaatcgtggtgaatgttaattaactcgaggctgagcaaagcagactactaataacataaagtctacgccggacgcatcgtggccctagtacgcaagttcacgtaaaaagggtaactagaggttgaggtgattttatggaaattagcgaagttcagctggttgaaagcggtggtggtctggttcagcctggtggtagcctgcgtctgagctgtgcagcaagcggttttaacattaaagatacctatatccactgggttcgtcaggcaccgggtaaaggtctggaatgggttgcacgtatttatccgaccaatggttatacccgttatgccgatagcgttaaaggtcgttttaccattagcgcagataccagcaaaaataccgcatacctgcagatgaatagtctgcgtgcagaggataccgcagtgtattattgtagccgttggggtggtgatggtttttatgcaatggattattggggtcagggcaccctggttaccgttagcagcgcaagcaccaaaggtccgagcgtttttccgctggcaccgagcagcaaaagcaccagcggtggcaccgcagcactgggttgtctggttaaagattattttccggaaccggttaccgtgagctggaatagcggtgcactgaccagtggtgttcatacctttccggcagttctgcagagcagcggtctgtatagtctgagcagcgttgttaccgttccgagcagtagcctgggcacccagacctatatttgtaatgttaatcataaaccgagcaacaccaaagtggataaaaaagttgaaccgaaaagctgcgataaaacccataccggtggtggtagcggtcatcatcaccaccatcaccatcatggtggtggcagtggtggtagtatgctgtttcgtgttaccattaacagctaa

SEQ ID NO: 24 - protein, 217 aa, synthetic construct; identical to SEQ ID NO: 18
  REGION 1..217: note = light chain

SEQ ID NO: 25 - protein, 261 aa, synthetic construct
  REGION 1..261: note = heavy chain-His Tag-Beta9
MEISEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGSGHHHHHHHHGGGSGGSMLFRVTINS

SEQ ID NO: 26 - DNA, 1555 bp, synthetic construct
  misc_feature 1..1555: note = TrasH-Beta10*
  source 1..1555: mol_type = other DNA
atggcaagcgatattcagatgacccagagcccgagcagcctgagcgcaagcgttggtgatcgtgttaccattacctgtcgtgcaagccaggatgttaataccgcagttgcatggtatcagcagaaaccgggtaaagcaccgaaactgctgatttatagcgcaagctttctgtatagcggtgttccgagccgttttagcggtagccgtagcggcaccgattttaccctgaccattagcagcctgcagccggaagattttgcaacctattattgtcagcagcattacaccacccctccgacctttggtcagggcaccaaagttgaaattaaacgtaccgttgcagcaccgagcgtttttatctttccgcctagcgatgagcagctgaaaagcggtacagccagcgttgtttgtctgctgaataacttttatccgcgtgaagcaaaagttcagtggaaagttgataatgcactgcagagcggtaatagccaagaaagcgttaccgaacaggatagcaaagatagcacctatagcctgagcagcaccctgaccctgagcaaagcagattatgaaaaacacaaagtgtatgcctgcgaagttacccatcagggtctgagcagtccggttaccaaaagctttaatcgtggtgaatgttaattaactcgaggctgagcaaagcagactactaataacataaagtctacgccggacgcatcgtggccctagtacgcaagttcacgtaaaaagggtaactagaggttgaggtgattttatggaaattagcgaagttcagctggttgaaagcggtggtggtctggttcagcctggtggtagcctgcgtctgagctgtgcagcaagcggttttaacattaaagatacctatatccactgggttcgtcaggcaccgggtaaaggtctggaatgggttgcacgtatttatccgaccaatggttatacccgttatgccgatagcgttaaaggtcgttttaccattagcgcagataccagcaaaaataccgcatacctgcagatgaatagtctgcgtgcagaggataccgcagtgtattattgtagccgttggggtggtgatggtttttatgcaatggattattggggtcagggcaccctggttaccgttagcagcgcaagcaccaaaggtccgagcgtttttccgctggcaccgagcagcaaaagcaccagcggtggcaccgcagcactgggttgtctggttaaagattattttccggaaccggttaccgtgagctggaatagcggtgcactgaccagtggtgttcatacctttccggcagttctgcagagcagcggtctgtatagtctgagcagcgttgttaccgttccgagcagtagcctgggcacccagacctatatttgtaatgttaatcataaaccgagcaacaccaaagtggataaaaaagttgaaccgaaaagctgcgataaaacccataccggtggtggtagcggtcatcatcaccaccatcaccatcatggtggtggcagtggtgtgagcggttggcgtctgttcaaaaaaatcagctaa

SEQ ID NO: 27 - protein, 217 aa, synthetic construct; identical to SEQ ID NO: 18
  REGION 1..217: note = light chain

SEQ ID NO: 28 - protein, 261 aa, synthetic construct
  REGION 1..261: note = heavy chain-His Tag-Beta10*)
MEISEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGSGHHHHHHHHGGGSGVSGWRLFKKIS

SEQ ID NO: 29 - DNA, 1555 bp, synthetic construct
  misc_feature 1..1555: note = Beta9-LPert
  source 1..1555: mol_type = other DNA
atgggtagtatgctgtttcgtgttaccattaacagcggtggtggtagcggtcatcatcaccaccatcaccatcatggtggtggcagtggtatggcaagcgatattcagatgacccagagcccgagcagcctgagcgcaagcgttggtgatcgtgttaccattacctgtaaggcaagccaggatgttagcattggtgttgcatggtatcagcagaaaccgggtaaagcaccgaaactgctgatttatagcgcaagctatcgttataccggtgttccgagccgttttagcggtagcggcagcggcaccgattttaccctgaccattagcagcctgcagccggaagattttgcaacctattattgtcagcagtactacatctatccgtacacctttggtcagggcaccaaagttgaaattaaacgtaccgttgcagcaccgagcgtttttatcttcccgcctagcgatgagcagctgaaaagcggtacagccagcgttgtttgtctgctgaataacttttatccgcgtgaggcaaaagttcagtggaaagttgataatgcactgcagagcggtaatagccaagaaagcgttaccgaacaggatagcaaagatagcacctatagcctgagcagcaccctgaccctgagcaaagcagattatgaaaaacacaaagtgtatgcctgcgaagttacccatcagggtctgagcagtccggttaccaaaagctttaatcgtggtgaatgttaattaactcgaggctgagcaaagcagactactaataacataaagtctacgccggacgcatcgtggccctagtacgcaagttcacgtaaaaagggtaactagaggttgaggtgattttatggaaattagcgaagttcagctggttgaaagcggtggtggtctggttcagcctggtggtagcctgcgtctgagctgtgcagcaagcggttttacctttaccgattacaccatggattgggttcgtcaggcaccgggtaaaggtctggaatgggttgcagatgttaatccgaatagcggtggtagcatttataaccagcgttttaaaggtcgttttaccctgagcgttgatcgtagcaaaaataccctgtatctgcaaatgaatagtctgcgtgcagaggataccgcagtgtattattgtgcacgtaatctgggtccgagcttctattttgattattggggtcagggcaccctggttaccgttagcagcgcaagcaccaaaggtccgagcgtttttccgctggcaccgagcagcaaaagcaccagcggtggcaccgcagcactgggttgtctggttaaagattattttccggaaccggttaccgtgagctggaatagcggtgcactgaccagtggtgttcatacctttccggcagttctgcagagcagcggtctgtatagtctgagcagcgttgttaccgttccgagcagtagcctgggcacccagacctatatttgtaatgttaatcataaaccgagcaacaccaaagtggataaaaaagttgaaccgaaaagctgcgataaaacccatacctaa

SEQ ID NO: 30 - protein, 247 aa, synthetic construct
  REGION 1..247: note = Beta9-His Tag-light chain
MGSMLFRVTINSGGGSGHHHHHHHHGGGSGMASDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 231 aa, synthetic construct
  REGION 1..231: note = heavy chain
MEISEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 32 - DNA, 1555 bp, synthetic construct
  misc_feature 1..1555: note = Beta10*-LPert
  source 1..1555: mol_type = other DNA
atggtgagcggttggcgtctgttcaaaaaaatcagcggtggtggtagcggtcatcatcaccaccatcaccatcatggtggtggcagtggtatggcaagcgatattcagatgacccagagcccgagcagcctgagcgcaagcgttggtgatcgtgttaccattacctgtaaggcaagccaggatgttagcattggtgttgcatggtatcagcagaaaccgggtaaagcaccgaaactgctgatttatagcgcaagctatcgttataccggtgttccgagccgttttagcggtagcggcagcggcaccgattttaccctgaccattagcagcctgcagccggaagattttgcaacctattattgtcagcagtactacatctatccgtacacctttggtcagggcaccaaagttgaaattaaacgtaccgttgcagcaccgagcgtttttatctttccgcctagcgatgagcagctgaaaagcggtacagccagcgttgtttgtctgctgaataacttttatccgcgtgaagcaaaagttcagtggaaagttgataatgcactgcagagcggtaatagccaagaaagcgttaccgaacaggatagcaaagatagcacctatagcctgagcagcaccctgaccctgagcaaagcagattatgaaaaacacaaagtgtatgcctgcgaagttacccatcagggtctgagcagtccggttaccaaaagctttaatcgtggtgaatgttaattaactcgaggctgagcaaagcagactactaataacataaagtctacgccggacgcatcgtggccctagtacgcaagttcacgtaaaaagggtaactagaggttgaggtgattttatggaaattagcgaagttcagctggttgaaagcggtggtggtctggttcagcctggtggtagcctgcgtctgagctgtgcagcaagcggttttacctttaccgattacaccatggattgggttcgtcaggcaccgggtaaaggtctggaatgggttgcagatgttaatccgaatagcggtggtagcatttataaccagcgttttaaaggtcgttttaccctgagcgttgatcgtagcaaaaataccctgtatctgcaaatgaatagtctgcgtgcagaggataccgcagtgtattattgtgcacgtaatctgggtccgagcttctattttgattattggggtcagggcaccctggttaccgttagcagcgcaagcaccaaaggtccgagcgtttttccgctggcaccgagcagcaaaagcaccagcggtggcaccgcagcactgggttgtctggttaaagattattttccggaaccggttaccgtgagctggaatagcggtgcactgaccagtggtgttcatacctttccggcagttctgcagagcagcggtctgtatagtctgagcagcgttgttaccgttccgagcagtagcctgggcacccagacctatatttgtaatgttaatcataaaccgagcaacaccaaagtggataaaaaagttgaaccgaaaagctgcgataaaacccatacctaa

SEQ ID NO: 33 - protein, 247 aa, synthetic construct
  REGION 1..247: note = Beta10*-His Tag-light chain
MVSGWRLFKKISGGGSGHHHHHHHHGGGSGMASDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 231 aa, synthetic construct; identical to SEQ ID NO: 31
  REGION 1..231: note = heavy chain

SEQ ID NO: 35 - DNA, 1552 bp, synthetic construct
  misc_feature 1..1552: note = PertH-Beta9
  source 1..1552: mol_type = other DNA
atggcaagcgatattcagatgacccagagcccgagcagcctgagcgcaagcgttggtgatcgtgttaccattacctgtaaggcaagccaggatgttagcattggtgttgcatggtatcagcagaaaccgggtaaagcaccgaaactgctgatttatagcgcaagctatcgttataccggtgttccgagccgttttagcggtagcggcagcggcaccgattttaccctgaccattagcagcctgcagccggaagattttgcaacctattattgtcagcagtactacatctatccgtacacctttggtcagggcaccaaagttgaaattaaacgtaccgttgcagcaccgagcgtttttatctttccgcctagcgatgagcagctgaaaagcggtacagccagcgttgtttgtctgctgaataacttttatccgcgtgaagcaaaagttcagtggaaagttgataatgcactgcagagcggtaatagccaagaaagcgttaccgaacaggatagcaaagatagcacctatagcctgagcagcaccctgaccctgagcaaagcagattatgaaaaacacaaagtgtatgcctgcgaagttacccatcagggtctgagcagtccggttaccaaaagctttaatcgtggtgaatgttaattaactcgaggctgagcaaagcagactactaataacataaagtctacgccggacgcatcgtggccctagtacgcaagttcacgtaaaaagggtaactagaggttgaggtgattttatggaaattagcgaagttcagctggttgaaagcggtggtggtctggttcagcctggtggtagcctgcgtctgagctgtgcagcaagcggttttacctttaccgattacaccatggattgggttcgtcaggcaccgggtaaaggtctggaatgggttgcagatgttaatccgaatagcggtggtagcatttataaccagcgttttaaaggtcgttttaccctgagcgttgatcgtagcaaaaataccctgtatctgcaaatgaatagtctgcgtgcagaggataccgcagtgtattattgtgcacgtaatctgggtccgagcttctattttgattattggggtcagggcaccctggttaccgttagcagcgcaagcaccaaaggtccgagcgtttttccgctggcaccgagcagcaaaagcaccagcggtggcaccgcagcactgggttgtctggttaaagattattttccggaaccggttaccgtgagctggaatagcggtgcactgaccagtggtgttcatacctttccggcagttctgcagagcagcggtctgtatagtctgagcagcgttgttaccgttccgagcagtagcctgggcacccagacctatatttgtaatgttaatcataaaccgagcaacaccaaagtggataaaaaagttgaaccgaaaagctgcgataaaacccataccggtggtggtagcggtcatcatcaccaccatcaccatcatggtggtggcagtggtggtagtatgctgtttcgtgttaccattaacagctaa

SEQ ID NO: 36 - protein, 217 aa, synthetic construct
  REGION 1..217: note = light chain
MASDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 260 aa, synthetic construct
  REGION 1..260: note = heavy chain- His Tag-Beta9
MEISEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGSGHHHHHHHHGGGSGGSMLFRVTINS

SEQ ID NO: 38 - DNA, 1552 bp, synthetic construct
  misc_feature 1..1552: note = PertH-Beta10*
  source 1..1552: mol_type = other DNA
atggcaagcgatattcagatgacccagagcccgagcagcctgagcgcaagcgttggtgatcgtgttaccattacctgtaaggcaagccaggatgttagcattggtgttgcatggtatcagcagaaaccgggtaaagcaccgaaactgctgatttatagcgcaagctatcgttataccggtgttccgagccgttttagcggtagcggcagcggcaccgattttaccctgaccattagcagcctgcagccggaagattttgcaacctattattgtcagcagtactacatctatccgtacacctttggtcagggcaccaaagttgaaattaaacgtaccgttgcagcaccgagcgtttttatctttccgcctagcgatgagcagctgaaaagcggtacagccagcgttgtttgtctgctgaataacttttatccgcgtgaagcaaaagttcagtggaaagttgataatgcactgcagagcggtaatagccaagaaagcgttaccgaacaggatagcaaagatagcacctatagcctgagcagcaccctgaccctgagcaaagcagattatgaaaaacacaaagtgtatgcctgcgaagttacccatcagggtctgagcagtccggttaccaaaagctttaatcgtggtgaatgttaattaactcgaggctgagcaaagcagactactaataacataaagtctacgccggacgcatcgtggccctagtacgcaagttcacgtaaaaagggtaactagaggttgaggtgattttatggaaattagcgaagttcagctggttgaaagcggtggtggtctggttcagcctggtggtagcctgcgtctgagctgtgcagcaagcggttttacctttaccgattacaccatggattgggttcgtcaggcaccgggtaaaggtctggaatgggttgcagatgttaatccgaatagcggtggtagcatttataaccagcgttttaaaggtcgttttaccctgagcgttgatcgtagcaaaaataccctgtatctgcaaatgaatagtctgcgtgcagaggataccgcagtgtattattgtgcacgtaatctgggtccgagcttctattttgattattggggtcagggcaccctggttaccgttagcagcgcaagcaccaaaggtccgagcgtttttccgctggcaccgagcagcaaaagcaccagcggtggcaccgcagcactgggttgtctggttaaagattattttccggaaccggttaccgtgagctggaatagcggtgcactgaccagtggtgttcatacctttccggcagttctgcagagcagcggtctgtatagtctgagcagcgttgttaccgttccgagcagtagcctgggcacccagacctatatttgtaatgttaatcataaaccgagcaacaccaaagtggataaaaaagttgaaccgaaaagctgcgataaaacccataccggtggtggtagcggtcatcatcaccaccatcaccatcatggtggtggcagtggtgtgagcggttggcgtctgttcaaaaaaatcagctaa

SEQ ID NO: 39 - protein, 217 aa, synthetic construct; identical to SEQ ID NO: 36
  REGION 1..217: note = light chain

SEQ ID NO: 40 - protein, 260 aa, synthetic construct
  REGION 1..260: note = heavy chain-His Tag-Beta10*
MEISEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGSGHHHHHHHHGGGSGVSGWRLFKKIS

SEQ ID NO: 41 - DNA, 465 bp, synthetic construct
  misc_feature 1..465: note = Beta9-G3
  source 1..465: mol_type = other DNA
atgggtagtatgctgtttcgtgttaccattaacagcggtggtggtagcggtcatcatcaccaccatcaccatcatggtggtggcagtggtgatctgggtaaaaaactgctggaagcagcacgtgcaggtcaggatgatgaagttcgtattctgatggcaaatggtgcagatgttaatgccaaagatgaatatggtctgacaccgctgtatctggcaaccgcacatggtcatctggaaattgttgaagttctgctgaaaaacggtgccgatgtgaatgcagttgatgcaattggttttactcctctgcatctggcagcatttattggccacctggaaatcgccgaggtgctgctgaaacatggtgcggacgttaacgcacaggataaatttggtaaaaccgcctttgatattagcattggcaatggcaatgaagatctggcagaaatcctgcagaaactgaactaa

SEQ ID NO: 42 - protein, 154 aa, synthetic construct
  REGION 1..154: note = Beta9-His Tag-G3
MGSMLFRVTINSGGGSGHHHHHHHHGGGSGDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLN

SEQ ID NO: 43 - DNA, 465 bp, synthetic construct
  misc_feature 1..465: note = Beta10*-G3
  source 1..465: mol_type = other DNA
atggtgagcggttggcgtctgttcaaaaaaatcagcggtggtggtagcggtcatcatcaccaccatcaccatcatggtggtggcagtggtgatctgggtaaaaaactgctggaagcagcacgtgcaggtcaggatgatgaagttcgtattctgatggcaaatggtgcagatgttaatgccaaagatgaatatggtctgacaccgctgtatctggcaaccgcacatggtcatctggaaattgttgaagttctgctgaaaaacggtgccgatgtgaatgcagttgatgcaattggttttactcctctgcatctggcagcatttattggccacctggaaatcgccgaggtgctgctgaaacatggtgcggacgttaacgcacaggataaatttggtaaaaccgcctttgatattagcattggcaatggcaatgaagatctggcagaaatcctgcagaaactgaactaa

SEQ ID NO: 44 - protein, 154 aa, synthetic construct
  REGION 1..154: note = Beta10*-His Tag-G3
MVSGWRLFKKISGGGSGHHHHHHHHGGGSGDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLN

SEQ ID NO: 45 - DNA, 489 bp, synthetic construct
  misc_feature 1..489: note = G3-Beta9
  source 1..489: mol_type = other DNA
atgcgtggtagccatcatcatcaccatcatggtagcgatctgggtaaaaaactgctggaagcagcacgtgcaggtcaggatgatgaagttcgtattctgatggcaaatggtgcagatgttaatgccaaagatgaatatggtctgacaccgctgtatctggcaaccgcacatggtcatctggaaattgttgaagttctgctgaaaaacggtgccgatgtgaatgcagttgatgcaattggttttactcctctgcatctggcagcatttattggccacctggaaatcgccgaggtgctgctgaaacatggtgcggacgttaacgcacaggataaatttggtaaaaccgcctttgatattagcattggcaatggcaatgaagatctggcagaaatcctgcagaaactgaacggtggtggtagcggtggtggtggctcaggtggcggtggttctggtggtagtatgctgtttcgtgttaccattaacagctaa

SEQ ID NO: 46 - protein, 162 aa, synthetic construct
  REGION 1..162: note = His Tag-G3-Beta9
MRGSHHHHHHGSDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLNGGGSGGGGSGGGGSGGSMLFRVTINS

SEQ ID NO: 47 - DNA, 489 bp, synthetic construct
  misc_feature 1..489: note = G3-Beta10*
  source 1..489: mol_type = other DNA
atgcgtggtagccatcatcatcaccatcatggtagcgatctgggtaaaaaactgctggaagcagcacgtgcaggtcaggatgatgaagttcgtattctgatggcaaatggtgcagatgttaatgccaaagatgaatatggtctgacaccgctgtatctggcaaccgcacatggtcatctggaaattgttgaagttctgctgaaaaacggtgccgatgtgaatgcagttgatgcaattggttttactcctctgcatctggcagcatttattggccacctggaaatcgccgaggtgctgctgaaacatggtgcggacgttaacgcacaggataaatttggtaaaaccgcctttgatattagcattggcaatggcaatgaagatctggcagaaatcctgcagaaactgaacggtggtggtagcggtggtggtggctcaggtggcggtggttctggtgtgagcggttggcgtctgttcaaaaaaatcagctaa

SEQ ID NO: 48 - protein, 162 aa, synthetic construct
  REGION 1..162: note = His Tag-G3-Beta10*
MRGSHHHHHHGSDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLNGGGSGGGGSGGGGSGVSGWRLFKKIS

SEQ ID NO: 49 - DNA, 567 bp, synthetic construct
  misc_feature 1..567: note = Beta9-9.29
  source 1..567: mol_type = other DNA
atgggtagtatgctgtttcgtgttaccattaacagcggtggtggtagcggtcatcatcaccaccatcaccatcatggtggtggcagtggtggtagcgatctgggtaaaaaactgctggaagcagcacgtgcaggtcaggatgatgaagttcgtattctgatggcaaatggtgcagatgttaatgcccatgatttttatggtattacaccgctgcatctggcagcaaattttggtcatctggaaattgttgaagtgctgctgaaacatggtgccgatgtgaacgcatttgattatgataatacccctctgcacctggctgcagatgctggccacctggaaatcgtagaggttctgctgaaatacggtgcggacgttaatgcaagcgatcgtgatggtcataccccactgcatctggcagcccgtgaaggccatctggaaatcgtggaagtactgctgaaaaatggcgcagacgtgaatgcacaggataaatttggtaaaaccccgtttgatctggccattgataatggcaatgaagatattgccgaggtgctgcagaaagcagcataa

SEQ ID NO: 50 - protein, 188 aa, synthetic construct
  REGION 1..188: note = Beta9-His Tag-9.29
MGSMLFRVTINSGGGSGHHHHHHHHGGGSGGSDLGKKLLEAARAGQDDEVRILMANGADVNAHDFYGITPLHLAANFGHLEIVEVLLKHGADVNAFDYDNTPLHLAADAGHLEIVEVLLKYGADVNASDRDGHTPLHLAAREGHLEIVEVLLKNGADVNAQDKFGKTPFDLAIDNGNEDIAEVLQKAA

SEQ ID NO: 51 - DNA, 567 bp, synthetic construct
  misc_feature 1..567: note = Beta10*-9.29
  source 1..567: mol_type = other DNA
atggtgagcggttggcgtctgttcaaaaaaatcagcggtggtggtagcggtcatcatcaccaccatcaccatcatggtggtggcagtggtggtagcgatctgggtaaaaaactgctggaagcagcacgtgcaggtcaggatgatgaagttcgtattctgatggcaaatggtgcagatgttaatgcccatgatttttatggtattacaccgctgcatctggcagcaaattttggtcatctggaaattgttgaagtgctgctgaaacatggtgccgatgtgaacgcatttgattatgataatacccctctgcacctggctgcagatgctggccacctggaaatcgtagaggttctgctgaaatacggtgcggacgttaatgcaagcgatcgtgatggtcataccccactgcatctggcagcccgtgaaggccatctggaaatcgtggaagtactgctgaaaaatggcgcagacgtgaatgcacaggataaatttggtaaaaccccgtttgatctggccattgataatggcaatgaagatattgccgaggtgctgcagaaagcagcataa

SEQ ID NO: 52 - protein, 188 aa, synthetic construct
  REGION 1..188: note = Beta10*-His Tag-9.29
MVSGWRLFKKISGGGSGHHHHHHHHGGGSGGSDLGKKLLEAARAGQDDEVRILMANGADVNAHDFYGITPLHLAANFGHLEIVEVLLKHGADVNAFDYDNTPLHLAADAGHLEIVEVLLKYGADVNASDRDGHTPLHLAAREGHLEIVEVLLKNGADVNAQDKFGKTPFDLAIDNGNEDIAEVLQKAA

SEQ ID NO: 53 - DNA, 567 bp, synthetic construct
  misc_feature 1..567: note = 9.29-Beta9
  source 1..567: mol_type = other DNA
atgggtagcgatctgggtaaaaaactgctggaagcagcacgtgcaggtcaggatgatgaagttcgtattctgatggcaaatggtgcagatgttaatgcccatgatttttatggtattacaccgctgcatctggcagcaaattttggtcatctggaaattgttgaagtgctgctgaaacatggtgccgatgtgaacgcatttgattatgataatacccctctgcacctggctgcagatgctggccacctggaaatcgtagaggttctgctgaaatacggtgcggacgttaatgcaagcgatcgtgatggtcataccccactgcatctggcagcccgtgaaggccatctggaaatcgtggaagtactgctgaaaaatggcgcagacgtgaatgcacaggataaatttggtaaaaccccgtttgatctggccattgataatggcaatgaagatattgccgaggtgctgcagaaagcagcaggtggtggtagcggtcatcatcaccaccatcaccatcatggtggtggcagtggtggtagtatgctgtttcgtgttaccattaacagctaa

SEQ ID NO: 54 - protein, 188 aa, synthetic construct
  REGION 1..188: note = 9.29-His Tag-Beta9
MGSDLGKKLLEAARAGQDDEVRILMANGADVNAHDFYGITPLHLAANFGHLEIVEVLLKHGADVNAFDYDNTPLHLAADAGHLEIVEVLLKYGADVNASDRDGHTPLHLAAREGHLEIVEVLLKNGADVNAQDKFGKTPFDLAIDNGNEDIAEVLQKAAGGGSGHHHHHHHHGGGSGGSMLFRVTINS

SEQ ID NO: 55 - DNA, 567 bp, synthetic construct
  misc_feature 1..567: note = 9.29-Beta10*
  source 1..567: mol_type = other DNA
atgggtagcgatctgggtaaaaaactgctggaagcagcacgtgcaggtcaggatgatgaagttcgtattctgatggcaaatggtgcagatgttaatgcccatgatttttatggtattacaccgctgcatctggcagcaaattttggtcatctggaaattgttgaagtgctgctgaaacatggtgccgatgtgaacgcatttgattatgataatacccctctgcacctggctgcagatgctggccacctggaaatcgtagaggttctgctgaaatacggtgcggacgttaatgcaagcgatcgtgatggtcataccccactgcatctggcagcccgtgaaggccatctggaaatcgtggaagtactgctgaaaaatggcgcagacgtgaatgcacaggataaatttggtaaaaccccgtttgatctggccattgataatggcaatgaagatattgccgaggtgctgcagaaagcagcaggtggtggtagcggtcatcatcaccaccatcaccatcatggtggtggcagtggtgtgagcggttggcgtctgttcaaaaaaatcagctaa

SEQ ID NO: 56 - protein, 188 aa, synthetic construct
  REGION 1..188: note = 9.29-His Tag-Beta10*
MGSDLGKKLLEAARAGQDDEVRILMANGADVNAHDFYGITPLHLAANFGHLEIVEVLLKHGADVNAFDYDNTPLHLAADAGHLEIVEVLLKYGADVNASDRDGHTPLHLAAREGHLEIVEVLLKNGADVNAQDKFGKTPFDLAIDNGNEDIAEVLQKAAGGGSGHHHHHHHHGGGSGVSGWRLFKKIS

SEQ ID NO: 57 - DNA, 1479 bp, synthetic construct
  misc_feature 1..1479: note = HT-Beta9-TNF
  source 1..1479: mol_type = other DNA
atgaaacatcatcaccatcaccacgcagaaatcggtactggctttccattcgacccccattatgtggaagtcctgggcgagcgcatgcactacgtcgatgttggtccgcgcgatggcacccctgtgctgttcctgcacggtaacccgacctcctcctacgtgtggcgcaacatcatcccgcatgttgcaccgacccatcgctgcattgctccagacctgatcggtatgggcaaatccgacaaaccagacctgggttatttcttcgacgaccacgtccgcttcatggatgccttcatcgaagccctgggtctggaagaggtcgtcctggtcattcacgactggggctccgctctgggtttccactgggccaagcgcaatccagagcgcgtcaaaggtattgcatttatggagttcatccgccctatcccgacctgggacgaatggccagaatttgcccgcgagaccttccaggccttccgcaccaccgacgtcggccgcaagctgatcatcgatcagaacgtttttatcgagggtacgctgccgatgggtgtcgtccgcccgctgactgaagtcgagatggaccattaccgcgagccgttcctgaatcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccaaaagcctgcctaactgcaaggctgtggacatcggcccgggtctgaatctgctgcaagaagacaacccggacctgatcggcagcgagatcgcgcgctggctgtcgacgctcgagatttccggcgagccaaccactgaggatctgtactttcagggtagtatgctgtttcgtgttaccattaacagcggtggtggtagcggtggtggtggcagtggtgttcgtagcagcagtcgtaccccgagcgataaaccggttgcacatgttgttgcaaatccgcaggccgaaggtcagctgcagtggctgaatcgtcgtgcaaatgcactgctggcaaatggtgttgaactgcgtgataatcagctggttgttccgagcgaaggtctgtatctgatttatagccaggttctgtttaaaggtcagggttgtccgagcacacatgttctgctgacccataccattagccgtattgcagttagctatcagaccaaagttaatctgctgagcgcaattaaaagcccgtgtcagcgtgaaacaccggaaggtgccgaagcaaaaccgtggtatgaaccgatttatcttggtggtgtttttcagctggaaaagggtgatcgtctgagcgcagaaattaatcgtccggattatctggattttgcagaaagcggtcaggtgtattttggcattattgcattataa

SEQ ID NO: 58 - protein, 492 aa, synthetic construct
  REGION 1..492: note = HT-Beta9-TNF
MKHHHHHHAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGEPTTEDLYFQGSMLFRVTINSGGGSGGGGSGVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 59 - DNA, 1482 bp, synthetic construct
  misc_feature 1..1482: note = HT-TNF-Beta9
  source 1..1482: mol_type = other DNA
atgaaacatcatcaccatcaccacgcagaaatcggtactggctttccattcgacccccattatgtggaagtcctgggcgagcgcatgcactacgtcgatgttggtccgcgcgatggcacccctgtgctgttcctgcacggtaacccgacctcctcctacgtgtggcgcaacatcatcccgcatgttgcaccgacccatcgctgcattgctccagacctgatcggtatgggcaaatccgacaaaccagacctgggttatttcttcgacgaccacgtccgcttcatggatgccttcatcgaagccctgggtctggaagaggtcgtcctggtcattcacgactggggctccgctctgggtttccactgggccaagcgcaatccagagcgcgtcaaaggtattgcatttatggagttcatccgccctatcccgacctgggacgaatggccagaatttgcccgcgagaccttccaggccttccgcaccaccgacgtcggccgcaagctgatcatcgatcagaacgtttttatcgagggtacgctgccgatgggtgtcgtccgcccgctgactgaagtcgagatggaccattaccgcgagccgttcctgaatcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccaaaagcctgcctaactgcaaggctgtggacatcggcccgggtctgaatctgctgcaagaagacaacccggacctgatcggcagcgagatcgcgcgctggctgtcgacgctcgagatttccggcgagccaaccactgaggatctgtactttcagggtgttcgtagcagcagtcgtaccccgagcgataaaccggttgcacatgttgttgcaaatccgcaggccgaaggtcagctgcagtggctgaatcgtcgtgcaaatgcactgctggcaaatggtgttgaactgcgtgataatcagctggttgttccgagcgaaggtctgtatctgatttatagccaggttctgtttaaaggtcagggttgtccgagcacacatgttctgctgacccataccattagccgtattgcagttagctatcagaccaaagttaatctgctgagcgcaattaaaagcccgtgtcagcgtgaaacaccggaaggtgccgaagcaaaaccgtggtatgaaccgatttatcttggtggtgtttttcagctggaaaagggtgatcgtctgagcgcagaaattaatcgtccggattatctggattttgcagaaagcggtcaggtgtattttggcattattgcattaggcggtggttcaggtggtggcggttctggtggtagtatgctgtttcgtgttaccattaacagctaa

SEQ ID NO: 60 - protein, 493 aa, synthetic construct
  REGION 1..493: note = HT-TNF-Beta9
MKHHHHHHAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGEPTTEDLYFQGVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGGSGGGGSGGSMLFRVTINS

SEQ ID NO: 61 - DNA, 1482 bp, synthetic construct
  misc_feature 1..1482: note = HT-Beta10*-TNF
  source 1..1482: mol_type = other DNA
atgaaacatcatcaccatcaccacgcagaaatcggtactggctttccattcgacccccattatgtggaagtcctgggcgagcgcatgcactacgtcgatgttggtccgcgcgatggcacccctgtgctgttcctgcacggtaacccgacctcctcctacgtgtggcgcaacatcatcccgcatgttgcaccgacccatcgctgcattgctccagacctgatcggtatgggcaaatccgacaaaccagacctgggttatttcttcgacgaccacgtccgcttcatggatgccttcatcgaagccctgggtctggaagaggtcgtcctggtcattcacgactggggctccgctctgggtttccactgggccaagcgcaatccagagcgcgtcaaaggtattgcatttatggagttcatccgccctatcccgacctgggacgaatggccagaatttgcccgcgagaccttccaggccttccgcaccaccgacgtcggccgcaagctgatcatcgatcagaacgtttttatcgagggtacgctgccgatgggtgtcgtccgcccgctgactgaagtcgagatggaccattaccgcgagccgttcctgaatcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccaaaagcctgcctaactgcaaggctgtggacatcggcccgggtctgaatctgctgcaagaagacaacccggacctgatcggcagcgagatcgcgcgctggctgtcgacgctcgagatttccggcgagccaaccactgaggatctgtactttcagggtgtgagcggttggcgtctgttcaaaaaaatcagcggtggtggtagcggtggtggtggcagtggtgttcgtagcagcagtcgtaccccgagcgataaaccggttgcacatgttgttgcaaatccgcaggccgaaggtcagctgcagtggctgaatcgtcgtgcaaatgcactgctggcaaatggtgttgaactgcgtgataatcagctggttgttccgagcgaaggtctgtatctgatttatagccaggttctgtttaaaggtcagggttgtccgagcacacatgttctgctgacccataccattagccgtattgcagttagctatcagaccaaagttaatctgctgagcgcaattaaaagcccgtgtcagcgtgaaacaccggaaggtgccgaagcaaaaccgtggtatgaaccgatttatcttggtggtgtttttcagctggaaaagggtgatcgtctgagcgcagaaattaatcgtccggattatctggattttgcagaaagcggtcaggtgtattttggcattattgcattataa

SEQ ID NO: 62 - protein, 493 aa, synthetic construct
  REGION 1..493: note = HT-Beta10*-TNF
MKHHHHHHAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGEPTTEDLYFQGVSGWRLFKKISGGGSGGGGSGVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 63 - DNA, 1482 bp, synthetic construct
  misc_feature 1..1482: note = HT-TNF-Beta10*
  source 1..1482: mol_type = other DNA
atgaaacatcatcaccatcaccacgcagaaatcggtactggctttccattcgacccccattatgtggaagtcctgggcgagcgcatgcactacgtcgatgttggtccgcgcgatggcacccctgtgctgttcctgcacggtaacccgacctcctcctacgtgtggcgcaacatcatcccgcatgttgcaccgacccatcgctgcattgctccagacctgatcggtatgggcaaatccgacaaaccagacctgggttatttcttcgacgaccacgtccgcttcatggatgccttcatcgaagccctgggtctggaagaggtcgtcctggtcattcacgactggggctccgctctgggtttccactgggccaagcgcaatccagagcgcgtcaaaggtattgcatttatggagttcatccgccctatcccgacctgggacgaatggccagaatttgcccgcgagaccttccaggccttccgcaccaccgacgtcggccgcaagctgatcatcgatcagaacgtttttatcgagggtacgctgccgatgggtgtcgtccgcccgctgactgaagtcgagatggaccattaccgcgagccgttcctgaatcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccaaaagcctgcctaactgcaaggctgtggacatcggcccgggtctgaatctgctgcaagaagacaacccggacctgatcggcagcgagatcgcgcgctggctgtcgacgctcgagatttccggcgagccaaccactgaggatctgtactttcagggtgttcgtagcagcagtcgtaccccgagcgataaaccggttgcacatgttgttgcaaatccgcaggccgaaggtcagctgcagtggctgaatcgtcgtgcaaatgcactgctggcaaatggtgttgaactgcgtgataatcagctggttgttccgagcgaaggtctgtatctgatttatagccaggttctgtttaaaggtcagggttgtccgagcacacatgttctgctgacccataccattagccgtattgcagttagctatcagaccaaagttaatctgctgagcgcaattaaaagcccgtgtcagcgtgaaacaccggaaggtgccgaagcaaaaccgtggtatgaaccgatttatcttggtggtgtttttcagctggaaaagggtgatcgtctgagcgcagaaattaatcgtccggattatctggattttgcagaaagcggtcaggtgtattttggcattattgcattaggcggtggttcaggtggtggcggttctggtgttagcggttggcgtctgtttaaaaagattagctaa

SEQ ID NO: 64 - protein, 493 aa, synthetic construct
  REGION 1..493: note = HT-TNF-Beta10*
MKHHHHHHAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGEPTTEDLYFQGVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGGSGGGGSGVSGWRLFKKIS

SEQ ID NO: 65 - DNA, 564 bp, synthetic construct
  misc_feature 1..564: note = Beta9-TNF
  source 1..564: mol_type = other DNA
atgggtagtatgctgtttcgtgttaccattaacagcggtggtggtagcggtcatcatcaccaccatcaccatcatggtggtggcagtggtgttcgtagcagcagtcgtaccccgagcgataaaccggttgcacatgttgttgcaaatccgcaggccgaaggtcagctgcagtggctgaatcgtcgtgcaaatgcactgctggcaaatggtgttgaactgcgtgataatcagctggttgttccgagcgaaggtctgtatctgatttatagccaggttctgtttaaaggtcagggttgtccgagcacacatgttctgctgacccataccattagccgtattgcagttagctatcagaccaaagttaatctgctgagcgcaattaaaagcccgtgtcagcgtgaaacaccggaaggtgccgaagcaaaaccgtggtatgaaccgatttatcttggtggtgtttttcagctggaaaagggtgatcgtctgagcgcagaaattaatcgtccggattatctggattttgcagaaagcggtcaggtgtattttggcattattgcattataa

SEQ ID NO: 66 - protein, 187 aa, synthetic construct
  REGION 1..187: note = Beta9-TNF
MGSMLFRVTINSGGGSGHHHHHHHHGGGSGVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 67 - DNA, 564 bp, synthetic construct
  misc_feature 1..564: note = TNF-Beta9
  source 1..564: mol_type = other DNA
atggttcgtagcagcagtcgtaccccgagcgataaaccggttgcacatgttgttgcaaatccgcaggccgaaggtcagctgcagtggctgaatcgtcgtgcaaatgcactgctggcaaatggtgttgaactgcgtgataatcagctggttgttccgagcgaaggtctgtatctgatttatagccaggttctgtttaaaggtcagggttgtccgagcacacatgttctgctgacccataccattagccgtattgcagttagctatcagaccaaagttaatctgctgagcgcaattaaaagcccgtgtcagcgtgaaacaccggaaggtgccgaagcaaaaccgtggtatgaaccgatttatcttggtggtgtttttcagctggaaaagggtgatcgtctgagcgcagaaattaatcgtccggattatctggattttgcagaaagcggtcaggtgtattttggcattattgcattaggtggtggtagcggtcatcatcaccaccatcaccatcatggtggtggcagtggtggtagtatgctgtttcgtgttaccattaacagctaa

SEQ ID NO: 68 - protein, 187 aa, synthetic construct
  REGION 1..187: note = TNF-Beta9
MVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGGSGHHHHHHHHGGGSGGSMLFRVTINS

SEQ ID NO: 69 - DNA, 564 bp, synthetic construct
  misc_feature 1..564: note = Beta10*-TNF
  source 1..564: mol_type = other DNA
atggtgagcggttggcgtctgttcaaaaaaatcagcggtggtggtagcggtcatcatcaccaccatcaccatcatggtggtggcagtggtgttcgtagcagcagtcgtaccccgagcgataaaccggttgcacatgttgttgcaaatccgcaggccgaaggtcagctgcagtggctgaatcgtcgtgcaaatgcactgctggcaaatggtgttgaactgcgtgataatcagctggttgttccgagcgaaggtctgtatctgatttatagccaggttctgtttaaaggtcagggttgtccgagcacacatgttctgctgacccataccattagccgtattgcagttagctatcagaccaaagttaatctgctgagcgcaattaaaagcccgtgtcagcgtgaaacaccggaaggtgccgaagcaaaaccgtggtatgaaccgatttatcttggtggtgtttttcagctggaaaagggtgatcgtctgagcgcagaaattaatcgtccggattatctggattttgcagaaagcggtcaggtgtattttggcattattgcattataa

SEQ ID NO: 70 - protein, 187 aa, synthetic construct
  REGION 1..187: note = Beta10*-TNF
MVSGWRLFKKISGGGSGHHHHHHHHGGGSGVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 71 - DNA, 564 bp, synthetic construct
  misc_feature 1..564: note = TNF-Beta10*
  source 1..564: mol_type = other DNA
atggttcgtagcagcagtcgtaccccgagcgataaaccggttgcacatgttgttgcaaatccgcaggccgaaggtcagctgcagtggctgaatcgtcgtgcaaatgcactgctggcaaatggtgttgaactgcgtgataatcagctggttgttccgagcgaaggtctgtatctgatttatagccaggttctgtttaaaggtcagggttgtccgagcacacatgttctgctgacccataccattagccgtattgcagttagctatcagaccaaagttaatctgctgagcgcaattaaaagcccgtgtcagcgtgaaacaccggaaggtgccgaagcaaaaccgtggtatgaaccgatttatcttggtggtgtttttcagctggaaaagggtgatcgtctgagcgcagaaattaatcgtccggattatctggattttgcagaaagcggtcaggtgtattttggcattattgcattaggtggtggtagcggtcatcatcaccaccatcaccatcatggtggtggcagtggtgtgagcggttggcgtctgttcaaaaaaatcagctaa

SEQ ID NO: 72 - protein, 187 aa, synthetic construct
  REGION 1..187: note = TNF-Beta10*
MVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGGSGHHHHHHHHGGGSGVSGWRLFKKIS

SEQ ID NO: 73 - DNA, 282 bp, synthetic construct
  misc_feature 1..282: note = Beta9-PpL
  source 1..282: mol_type = other DNA
atgggtagtatgctgtttcgtgttaccattaacagcggtggtggtagcggtcatcatcaccaccatcaccatcatggtggtggcagtggtagcgaagtgaccattaaagtgaacctgatttttgcggatggcaaaattcagaccgcagaatttaaaggcacctttgaagaagcaacagccgaagcatatcgttatgcagcactgctggcaaaagttaatggtgaatataccgcagatctggaagatggtggtaatcacatgaatatcaaattcgcaggctaa

SEQ ID NO: 74 - protein, 93 aa, synthetic construct
  REGION 1..93: note = Beta9-PpL
MGSMLFRVTINSGGGSGHHHHHHHHGGGSGSEVTIKVNLIFADGKIQTAEFKGTFEEATAEAYRYAALLAKVNGEYTADLEDGGNHMNIKFAG

SEQ ID NO: 75 - DNA, 282 bp, synthetic construct
  misc_feature 1..282: note = PpL-Beta9
  source 1..282: mol_type = other DNA
atgagcgaagtgaccattaaagtgaacctgatttttgcggatggcaaaattcagaccgcagaatttaaaggcacctttgaagaagcaacagccgaagcatatcgttatgcagcactgctggcaaaagttaatggtgaatataccgcagatctggaagatggtggtaatcacatgaatatcaaattcgcaggcggtggtggtagcggtcatcatcaccaccatcaccatcatggtggtggcagtggtggtagtatgctgtttcgtgttaccattaacagctaa

SEQ ID NO: 76 - protein, 93 aa, synthetic construct
  REGION 1..93: note = PpL-Beta9
MSEVTIKVNLIFADGKIQTAEFKGTFEEATAEAYRYAALLAKVNGEYTADLEDGGNHMNIKFAGGGGSGHHHHHHHHGGGSGGSMLFRVTINS

SEQ ID NO: 77 - DNA, 282 bp, synthetic construct
  misc_feature 1..282: note = Beta10-PpL
  source 1..282: mol_type = other DNA
atggtgagcggttggcgtctgttcaaaaaaatcagcggtggtggtagcggtcatcatcaccaccatcaccatcatggtggtggcagtggtagcgaagtgaccattaaagtgaacctgatttttgcggatggcaaaattcagaccgcagaatttaaaggcacctttgaagaagcaacagccgaagcatatcgttatgcagcactgctggcaaaagttaatggtgaatataccgcagatctggaagatggtggtaatcacatgaatatcaaattcgcaggctaa

SEQ ID NO: 78 - protein, 93 aa, synthetic construct
  REGION 1..93: note = Beta10-PpL
MVSGWRLFKKISGGGSGHHHHHHHHGGGSGSEVTIKVNLIFADGKIQTAEFKGTFEEATAEAYRYAALLAKVNGEYTADLEDGGNHMNIKFAG

SEQ ID NO: 79 - DNA, 282 bp, synthetic construct
  misc_feature 1..282: note = PpL-Beta10*
  source 1..282: mol_type = other DNA
atgagcgaagtgaccattaaagtgaacctgatttttgcggatggcaaaattcagaccgcagaatttaaaggcacctttgaagaagcaacagccgaagcatatcgttatgcagcactgctggcaaaagttaatggtgaatataccgcagatctggaagatggtggtaatcacatgaatatcaaattcgcaggcggtggtggtagcggtcatcatcaccaccatcaccatcatggtggtggcagtggtgtgagcggttggcgtctgttcaaaaaaatcagctaa

SEQ ID NO: 80 - protein, 93 aa, synthetic construct
  REGION 1..93: note = PpL-Beta10*
MSEVTIKVNLIFADGKIQTAEFKGTFEEATAEAYRYAALLAKVNGEYTADLEDGGNHMNIKFAGGGGSGHHHHHHHHGGGSGVSGWRLFKKIS

SEQ ID NO: 81 - DNA, 258 bp, synthetic construct
  misc_feature 1..258: note = Beta9-SpA
  source 1..258: mol_type = other DNA
atgggtagtatgctgtttcgtgttaccattaacagcggtggtggtagcggtcatcatcaccaccatcaccatcatggtggtggcagtggtagctacaacaaagatcagcagagcgccttttatgaaattctgaatatgccgaatctgaatgaagcacagcgcaatggttttattcagagcctgaaagatgatccgagccagagcaccaatgttctgggtgaagcaaaaaaactgaatgaaagccaggcaccgaaataa

SEQ ID NO: 82 - protein, 85 aa, synthetic construct
  REGION 1..85: note = Beta9-SpA
MGSMLFRVTINSGGGSGHHHHHHHHGGGSGSYNKDQQSAFYEILNMPNLNEAQRNGFIQSLKDDPSQSTNVLGEAKKLNESQAPK

SEQ ID NO: 83 - DNA, 258 bp, synthetic construct
  misc_feature 1..258: note = SpA-Beta9
  source 1..258: mol_type = other DNA
atgagctacaacaaagatcagcagagcgccttttatgaaattctgaatatgccgaatctgaatgaagcacagcgcaatggttttattcagagcctgaaagatgatccgagccagagcaccaatgttctgggtgaagcaaaaaaactgaatgaaagccaggcaccgaaaggtggtggtagcggtcatcatcaccaccatcaccatcatggtggtggcagtggtggtagtatgctgtttcgtgttaccattaacagctaa

SEQ ID NO: 84 - protein, 85 aa, synthetic construct
  REGION 1..85: note = SpA-Beta9
MSYNKDQQSAFYEILNMPNLNEAQRNGFIQSLKDDPSQSTNVLGEAKKLNESQAPKGGGSGHHHHHHHHGGGSGGSMLFRVTINS

SEQ ID NO: 85 - DNA, 258 bp, synthetic construct
  misc_feature 1..258: note = Beta10*-SpA
  source 1..258: mol_type = other DNA
atggtgagcggttggcgtctgttcaaaaaaatcagcggtggtggtagcggtcatcatcaccaccatcaccatcatggtggtggcagtggtagctacaacaaagatcagcagagcgccttttatgaaattctgaatatgccgaatctgaatgaagcacagcgcaatggttttattcagagcctgaaagatgatccgagccagagcaccaatgttctgggtgaagcaaaaaaactgaatgaaagccaggcaccgaaataa

SEQ ID NO: 86 - protein, 85 aa, synthetic construct
  REGION 1..85: note = Beta10*-SpA
MVSGWRLFKKISGGGSGHHHHHHHHGGGSGSYNKDQQSAFYEILNMPNLNEAQRNGFIQSLKDDPSQSTNVLGEAKKLNESQAPK

SEQ ID NO: 87 - DNA, 258 bp, synthetic construct
  misc_feature 1..258: note = SpA-Beta10*
  source 1..258: mol_type = other DNA
atgagctacaacaaagatcagcagagcgccttttatgaaattctgaatatgccgaatctgaatgaagcacagcgcaatggttttattcagagcctgaaagatgatccgagccagagcaccaatgttctgggtgaagcaaaaaaactgaatgaaagccaggcaccgaaaggtggtggtagcggtcatcatcaccaccatcaccatcatggtggtggcagtggtgtgagcggttggcgtctgttcaaaaaaatcagctaa

SEQ ID NO: 88 - protein, 85 aa, synthetic construct
  REGION 1..85: note = SpA-Beta10*
MSYNKDQQSAFYEILNMPNLNEAQRNGFIQSLKDDPSQSTNVLGEAKKLNESQAPKGGGSGHHHHHHHHGGGSGVSGWRLFKKIS

SEQ ID NO: 89 - DNA, 267 bp, synthetic construct
  misc_feature 1..267: note = Beta9-SpGA1
  source 1..267: mol_type = other DNA
atgggtagtatgctgtttcgtgttaccattaacagcggtggtggtagcggtcatcatcaccaccatcaccatcatggtggtggcagtggtgcagtgaccacctataaactggttattaatggtcgtaccctgagcggttataccaccaccaccgcagttgatgcagaaaccgcagaaaaagcatttaaacagtatgcctatgtgcatgaagttgatggtgtttggacctatgatgatgcaaccaaaacctttaccgttaccgaataa

SEQ ID NO: 90 - protein, 88 aa, synthetic construct
  REGION 1..88: note = Beta9-SpGA1
MGSMLFRVTINSGGGSGHHHHHHHHGGGSGAVTTYKLVINGRTLSGYTTTTAVDAETAEKAFKQYAYVHEVDGVWTYDDATKTFTVTE

SEQ ID NO: 91 - DNA, 267 bp, synthetic construct
  misc_feature 1..267: note = SpGA1-Beta9
  source 1..267: mol_type = other DNA
atggcagtgaccacctataaactggttattaatggtcgtaccctgagcggttataccaccaccaccgcagttgatgcagaaaccgcagaaaaagcatttaaacagtatgcctatgtgcatgaagttgatggtgtttggacctatgatgatgcaaccaaaacctttaccgttaccgaaggtggtggtagcggtcatcatcaccaccatcaccatcatggtggtggcagtggtggtagtatgctgtttcgtgttaccattaacagctaa

SEQ ID NO: 92 - protein, 88 aa, synthetic construct
  REGION 1..88: note = SpGA1-Beta9
MAVTTYKLVINGRTLSGYTTTTAVDAETAEKAFKQYAYVHEVDGVWTYDDATKTFTVTEGGGSGHHHHHHHHGGGSGGSMLFRVTINS

SEQ ID NO: 93 - DNA, 267 bp, synthetic construct
  misc_feature 1..267: note = Beta10*-SpGA1
  source 1..267: mol_type = other DNA
atggtgagcggttggcgtctgttcaaaaaaatcagcggtggtggtagcggtcatcatcaccaccatcaccatcatggtggtggcagtggtgcagtgaccacctataaactggttattaatggtcgtaccctgagcggttataccaccaccaccgcagttgatgcagaaaccgcagaaaaagcatttaaacagtatgcctatgtgcatgaagttgatggtgtttggacctatgatgatgcaaccaaaacctttaccgttaccgaataa

SEQ ID NO: 94 - protein, 88 aa, synthetic construct
  REGION 1..88: note = Beta10*-SpGA1
MVSGWRLFKKISGGGSGHHHHHHHHGGGSGAVTTYKLVINGRTLSGYTTTTAVDAETAEKAFKQYAYVHEVDGVWTYDDATKTFTVTE

SEQ ID NO: 95 - DNA, 267 bp, synthetic construct
  misc_feature 1..267: note = SpGA1-Beta10
  source 1..267: mol_type = other DNA
atggcagtgaccacctataaactggttattaatggtcgtaccctgagcggttataccaccaccaccgcagttgatgcagaaaccgcagaaaaagcatttaaacagtatgcctatgtgcatgaagttgatggtgtttggacctatgatgatgcaaccaaaacctttaccgttaccgaaggtggtggtagcggtcatcatcaccaccatcaccatcatggtggtggcagtggtgtgagcggttggcgtctgttcaaaaaaatcagctaa

SEQ ID NO: 96 - protein, 88 aa, synthetic construct
  REGION 1..88: note = SpGA1-Beta10*
MAVTTYKLVINGRTLSGYTTTTAVDAETAEKAFKQYAYVHEVDGVWTYDDATKTFTVTEGGGSGHHHHHHHHGGGSGVSGWRLFKKIS

(19) United States
(12) Patent Application Publication
(10) Pub. No.: US 2024/0210406 A1
(43) Pub. Date: Jun. 27, 2024

(54) TARGET-BINDING ACTIVATED SPLIT REPORTER SYSTEMS FOR ANALYTE DETECTION AND RELATED COMPONENTS AND METHODS

(71) Applicant: UNIVERSITY OF UTAH RESEARCH FOUNDATION, Salt Lake City, UT (US)

(72) Inventors: Andrew S. Dixon, Salt Lake City, UT (US); Shawn Owen, Salt Lake City, UT (US)

(21) Appl. No.: 18/453,745

(22) Filed: Aug. 22, 2023

Related U.S. Application Data
(63) Continuation of application No. 16/840,713, filed on Apr. 6, 2020, now Pat. No. 11,774,453.
Application No. 16/840,713 is a continuation of application No. 15/498,291, filed on Apr. 26, 2017, now Pat. No. 10,634,680.
(60) Provisional application No. 62/327,920, filed on Apr. 26, 2016.

(51) Int. Cl. G01N 33/574; C07K 16/24; C12Q 1/66; C12Q 1/6804; G01N 33/542; G01N 33/58

(52) U.S. Cl. CPC G01N 33/57488; C07K 16/241; C12Q 1/66; G01N 33/542; G01N 33/581; C07K 2317/24; C12Q 1/6804; G01N 2333/525

(57) ABSTRACT

Split enzyme reporter systems are disclosed for detecting an analyte in a mixture. Fragments of the split enzyme may be covalently bound to targeting domains that bind to target regions of an analyte, thereby causing formation of an active complex. Some split enzyme reporter systems can be used to detect an analyte without the use of analyte immobilization, blocking, or wash steps. Some reporter systems also enable rapid detection of the analyte of interest.

### RELATED APPLICATIONS

This application claims the benefit of U.S. Provisional Application No. 62/327,920, filed on Apr. 26, 2016 and titled “TARGET-BINDING ACTIVATED SPLIT REPORTER SYSTEMS FOR ANALYTE DETECTION AND RELATED COMPONENTS AND METHODS,” which is hereby incorporated by reference in its entirety.

### TECHNICAL FIELD

The present disclosure relates generally to the fields of biotechnology and analyte detection. More particularly, the present disclosure relates to split reporters, such as split reporter proteins that are configured to bind to and facilitate detection of an analyte or analytes in a mixture.

### BACKGROUND

Modern research and medicine involve qualitatively assessing and/or quantitatively measuring one or more analytes in a mixture. For example, in research settings, the identification and quantification of biomolecules, such as proteins, nucleic acids, sugars, lipids, etc., is important to understanding the mechanistic underpinnings of cellular processes. In clinical settings, the detection of biomarkers can enable the diagnosis of a disease, facilitate a more accurate prognosis for the patient, and/or provide a mechanism for monitoring therapeutic outcomes. As such, the ability to accurately and reliably detect analytes (e.g., biomolecules) in a timely manner is highly important.

For decades, researchers have relied on various immunoassays, such as ELISAs, western blots, immunocytochemistry, and/or immunohistochemistry for biomolecule (e.g., protein) detection. Such immunoassays may be time-consuming (e.g., >6 hours) and labor-intensive. One factor contributing to the labor and time required for some immunoassays is the need to remove excess (i.e., unbound) antibodies. For example, some immunoassays require numerous wash steps before and after addition of a primary antibody and a secondary antibody. Some immunoassays additionally or alternatively require one or more blocking steps and/or immobilization of the analyte. Some immunoassays cannot be carried out in a homogeneous solution.

Split proteins have been used for the detection and/or quantification of protein interactions. Various names have been given to the processes used for such detection and/or quantification, such as protein-fragment complementation assays (Michnick et al., Nat Rev Drug Discov 6, 569-82 (2007); Remy & Michnick, Methods Mol Biol 1278, 467-81 (2015)), split protein complementation (Shekhawat & Ghosh, Curr Opin Chem Biol 15, 789-97 (2011)), or bimolecular fluorescence complementation (Miller et al., J Mol Biol 427, 2039-55 (2015); Kerppola, T. K., Chem Soc Rev 38, 2876-2886 (2009)). In these split protein systems, each fragment of the split protein is individually inactive. However, when the fragments of a split protein are combined at high concentrations, the fragments can form an active protein complex. This ability to turn on the activity of the split protein can be exploited to monitor protein interactions by fusing each peptide fragment of the split protein to different proteins that have affinity for one another. The interaction between these different proteins creates a high local concentration of the two peptide fragments, thereby causing the separate fragments of the split protein to bind to one another to form an active protein complex.

Several split proteins have been used in complementation assays, including β-lactamase, β-galactosidase, dihydrofolate reductase, green fluorescent protein, ubiquitin, and TEV protease (Morrell et al., FEBS Lett 583, 1684-91 (2009). One split protein that has been used to detect and quantify protein interactions is NanoBiT® (Promega®). NanoBiT® is a split and modified form of NanoLuc® (Promega®), an engineered luciferase derived from a deep sea luminous shrimp (Dixon et al., ACS Chem Biol 11, 400-08 (2016)). The split NanoBiT® enzyme includes a relatively short peptide fragment (11 amino acids) and a relatively long peptide fragment (an 18 kDa polypeptide).

### BRIEF DESCRIPTION OF THE DRAWINGS

The written disclosure herein describes illustrative embodiments that are non-limiting and non-exhaustive. Reference is made to certain of such illustrative embodiments that are depicted in the figures, in which:

FIG. 1 is a schematic representation of components of a target-binding activated split reporter system.

FIG. 2 is a schematic that shows alternative targeting modes for activating complementation in a split reporter system.

FIG. 3 is a schematic showing binding of fragment complementation components to an immunoglobulin antibody.

FIG. 4 is a schematic showing binding of fragment complementation components to a modified protein.

FIG. 5 is a schematic representation of a tripartite split reporter assay for detecting an analyte in a mixture.

FIG. 6 is a schematic representation of a tripartite split reporter assay for detecting a molecular interaction.

FIG. 7A is a schematic representation of a tripartite split reporter assay for detecting an antibody.

FIG. 7B is a schematic representation of a tripartite split reporter assay for detecting an antibody.

FIG. 8 is a topological representation of an engineered split luciferase. (β10* is sometimes referred to as “86” in the drawings.)

FIG. 9 is a structural model of different binding states for a tripartite split reporter protein.

FIG. 10 is a bar graph showing luminescence of mixtures that have different components of a split reporter protein.

FIG. 11 is a scatterplot showing luminescence as a function of time for an analyte-detection assay.

FIG. 12 is a molecular model of HER2 bound to various targeting domains.

FIG. 13 is a bar chart showing luminescence of various targeted split enzyme reporter systems in the presence of a target antigen.

FIG. 14 is a bar chart showing luminescence of various targeted split enzyme reporter systems in the absence of a target antigen.

FIG. 15 is a bar graph depicting luminescent activity of various split enzyme reporter systems.

FIG. 16 is a bar graph depicting luminescent activity of various split enzyme systems.

FIG. 17 is a bar graph depicting luminescent activity of two targeted split enzyme reporter systems using purified fusions.

FIG. 18 is a plot showing luminescent activity as a function of the number of HER2⁺ cells.

FIG. 19 is a plot showing luminescent activity of a targeted split enzyme reporter system as a function of HER2 concentration.

FIG. 20 is a plot showing luminescent activity of another targeted split enzyme reporter system as a function of HER2 concentration.

FIG. 21 includes scatterplots that illustrate the binding kinetics of components of a targeted split enzyme reporter system.

FIG. 22 is a graph showing luminescent activity as a function of HER2 concentration in human serum.

FIG. 23 provides mass spectrometry data of a chemically conjugated agent that includes a peptide fragment and a targeting domain.

FIG. 24 is a bar graph showing luminescence from a targeted split enzyme reporter system that includes a chemically conjugated peptide fragment.

FIG. 25 is a scatterplot showing luminescent detection of IgG with a split enzyme reporter system having a first peptide fragment that is conjugated to Protein A and a second peptide fragment that is conjugated to Protein G.

FIG. 26 is a bar chart showing luminescence generated by various targeted split reporter systems.

FIG. 27 is a bar chart showing luminescence generated by various targeted split reporter systems.

FIG. 28 is a bar chart showing luminescence generated by various targeted split reporter systems.

FIG. 29 is a bar chart showing luminescence generated by various targeted split reporter systems.

FIG. 30 is a bar chart showing luminescence generated by various targeted split enzyme reporter systems.

FIG. 31 is a bar chart showing the signal-to-background ratio for luminescence generated by the targeted split enzyme reporter systems of FIG. 32.

FIG. 32 is a structural model showing the binding of protein A and tumor necrosis factor (TNF) to adalimumab.

FIG. 33 is a structural model showing the binding of protein L and tumor necrosis factor to adalimumab.

FIG. 34 is a bar chart showing both luminescence and the signal-to-background ratio for various split enzyme reporter systems.

### DETAILED DESCRIPTION

The following detailed description of various embodiments is not intended to limit the scope of the disclosure, but is merely representative of various embodiments. The present disclosure relates generally to split reporter systems, including split reporter systems for use in detecting and/or measuring an analyte.

“Enzymatic activity” includes the catalytic activity of a complex formed by fragment complementation. A “homomultimeric protein complex” includes homodimers, homotrimers, homotetramers, etc. A “heteromultimeric protein complex” includes heterodimers, heterotrimers, heterotetramers, etc. The term “domain” is not limited to protein domains. The term “peptide” or “peptide fragment” can refer to any chain of amino acids, regardless of length.

Immunoassays can be used to detect and/or quantify an analyte, such as a biomolecule. Many immunoassays require labor-intensive, time-consuming, multi-step protocols in order to remove unbound or non-specific targeting domains, thereby enabling specific detection due to binding of the analyte. Certain embodiments disclosed herein may provide advantages over known immunoassays, such as by decreasing the amount of labor (and thereby reducing the potential for human error), decreasing the time involved, decreasing the number of steps required to obtain a specific and/or measurable signal, and/or enabling detection/quantification under conditions that are not feasible with known immunoassays. Some embodiments can be used to monitor the presence, level, location, change in level, or change in location of an analyte.

Some embodiments disclosed herein involve the use of a split reporter protein, which may also be referred to as a split reporter or a split reporter protein complex. In some embodiments, the split reporter is a binary (i.e., two-part) reporter. In other embodiments, the split reporter is a ternary (i.e., three-part or tripartite) reporter. The individual fragments of the split reporter may be individually inactive. However, when combined with complementary peptide fragment(s) of the split reporter, the peptide fragments may bind to one another to form an active protein complex. Examples of split reporter proteins include split green fluorescent protein (Cabantous et al., Sci Rep 3, 2854 (2013)), NanoBiT®, and split β-lactamase. In some embodiments, the split reporter protein does not include a cysteine residue.

In some embodiments, the split reporter protein is a beta-barrel protein, such as a 10-stranded beta-barrel protein. In some embodiments, the split reporter protein is a split fluorescent protein, such as a split green fluorescent protein (e.g., a binary split fluorescent protein or a ternary split fluorescent protein). In other embodiments, the split reporter protein is a split enzyme. The split enzyme may catalyze the conversion of a substrate to a product. The activity of the split enzyme on the substrate may result in the emission of a detectable (e.g., luminescent) signal. For example, in some embodiments, the split enzyme is a split luciferase. In some embodiments, the substrate for the split enzyme is luciferin, furimazine, or some other luminogenic substrate or molecule. In some embodiments, the split enzyme catalyzes the conversion of furimazine to furimamide. In some embodiments, the enzymatic activity of the split enzyme is not natively found in mammals. In some embodiments, the split enzyme has no eukaryotic ortholog.

In some embodiments, the split reporter protein is a three-part (i.e., tripartite or ternary) complex that includes a first agent, a second agent, and a third agent. The first agent may include a first targeting domain and a first peptide fragment of a split reporter protein. The second agent may include a second targeting domain and a second peptide fragment of the split reporter protein. The third agent may include a third fragment of the split reporter protein.

The targeting domains of the first agent and the second agent may be any suitable targeting domain. For example, in some embodiments, the targeting domains of one or both of the first agent and the second agent comprise or consist of an antibody, an antigen, a designed ankyrin repeat protein (DARPin), an affibody, ubiquitin, a known interaction partner, a ligand, an aptamer, adnectin (monobody or Fibronectin type II domain), or a portion thereof. For example, in some embodiments, the targeting domains of one or both of the first agent and the second agent may be a fragment antigen binding fragment (Fab), a single-chain variable fragment, IgG, etc. In some embodiments, one or more targeting domains are monoclonal antibodies or portions thereof. In some embodiments, one or both targeting domains are polyclonal antibodies, or portions thereof. In some embodiments, one or more targeting domains are immunoglobulin-binding proteins, such as protein A, protein G, protein A/G, or protein L.

The first targeting domain may be configured to selectively bind to a first target region of an analyte, and the second targeting domain may be configured to selectively bind to a second target region of the analyte. For example, a first targeting domain may be configured to selectively bind to a first epitope of an analyte, while the second targeting domain is configured to selectively bind to a second epitope of the analyte. In some embodiments, the first target region and the second target region are different in structure (e.g., different epitopes). In other embodiments, the first target region and the second target region are identical or substantially identical in structure (e.g., identical or substantially identical epitopes), but are located at separate sites on the analyte (e.g., the protein). In some embodiments, the first target region and the second target region are separated by less than 300 (e.g., less than 150) angstroms. For example, in some embodiments, the first target region is separated from the second target region by 2-300 angstroms, 2-200 angstroms, 2-175 angstroms, 2-150 angstroms, 2-125 angstroms, 2-100 angstroms, 2-75 angstroms, 2-50 angstroms, 2-25 angstroms; 25-300 angstroms, 50-300 angstroms, 75-300 angstroms, 100-300 angstroms, 125-300 angstroms, 150-300 angstroms, 10-150 angstroms; 25-145 angstroms; 35-145 angstroms, 40-145 angstroms, 50-125 angstroms, or 60-100 angstroms. In some embodiments, the first and second target regions (e.g., epitopes) do not overlap.

The first peptide fragment and the second peptide fragment may be relatively short peptide fragments. Stated differently, one or both of the first peptide fragment and the second peptide fragment may have a mass of less than about 3 kDa. In some embodiments, the first peptide fragment is less than or equal to 15, 14, 13, 12, and/or 11 amino acids in length. In some embodiments, the second peptide fragment is less than or equal to 15, 14, 13, 12, and/or 11 amino acids in length. For example, in some embodiments, both the first peptide fragment and the second peptide fragment are each 11 amino acids in length. The relatively short length of the peptide fragments may facilitate expression, reduce aggregation, improve solubility, and/or improve the stability of agents that are produced as fusion proteins. Such small peptide fragments may also be less likely to interfere with binding of the targeting domain to the analyte.

In some embodiments, the first peptide fragment has at least 80%, 90%, or 100% sequence identity to the sequence of SEQ ID NO 1 (i.e., VSGWRLFKKIS). In some embodiments, the second peptide fragment has at least 80%, 90%, or 100% sequence identity to the sequence of SEQ ID NO 2 (i.e., GSMLFRVTINS).

In some embodiments, the first peptide fragment corresponds with a C-terminal beta sheet (β10) of a 10-stranded beta-barrel protein. In some embodiments, the second peptide fragment corresponds with the ninth beta sheet of a 10-stranded beta-barrel protein. In some embodiments, the first targeting domain of the first agent is positioned on the N-terminal side of a β10 fragment. In other embodiments, the first targeting domain of the first agent is positioned on the C-terminal side of a 10 fragment. In some embodiments, the second targeting domain of the second agent is positioned on the N-terminal side of a fragment corresponding to the ninth beta sheet. In other embodiments, the second targeting domain of the second agent is positioned on the C-terminal side of a fragment corresponding to the ninth beta sheet. In some embodiments, the peptide fragment and the targeting domain are connected via a linker. In some embodiments, the linker is a peptide. In other embodiments, the linker is not a peptide.

In some embodiments, one or both of the first agent and the second agent are recombinant fusion proteins. In some embodiments, the fusion proteins include a solubilizing protein or domain (e.g., HaloTag®, (Promega)). In other embodiments, one or both of the first agent and the second agent are formed by synthetically conjugating or enzymatically ligating a peptide fragment to a targeting domain. Such conjugation may facilitate the use of polyclonal antibodies or antibodies from hybridomas where the sequence of the targeting domain is not known. In some embodiments, a peptide fragment is conjugated to a targeting domain via an exposed sulfide (e.g., a cysteine residue) or an exposed amine (e.g., a lysine residue) on the targeting domain. In some embodiments, the targeting domain is modified to include a sulfhydryl group. For example, in some embodiments, 2-iminothiolane is used to modify a primary amine of a targeting domain to form an exposed sulfhydryl group. In some embodiments, a maleimide may be used to attach a peptide fragment to an exposed sulfhydryl group on a targeting domain (e.g., an antibody).

In some embodiments that include a third peptide fragment, the third peptide fragment has a mass of between 16 kDa and 17 kDa. In some embodiments, the third peptide fragment has at least 70%, 80%, 90%, 95% or 100% sequence identity to the sequence of SEQ ID NO 3. In some embodiments, the third peptide fragment has between 140 and 150 amino acids. For example, in some embodiments, the third peptide has between 145 and 150 amino acids (e.g., 148 amino acids).

The analyte to be detected and/or measured may be any suitable analyte. In some embodiments, the analyte is a biomolecule, such as a protein, nucleic acid, carbohydrate, or lipid. In some embodiments, the analyte has a monomeric quaternary structure (i.e., the analyte is not a dimer, trimer, etc.). In other embodiments, the analyte has a higher quaternary structure. For example, the analyte may be a dimer, a trimer, a tetramer, or any other multimeric complex. In some embodiments, the analyte is a modified protein, such as a phosphorylated protein, a glycosylated protein, or an antibody-drug conjugate (e.g., trastuzumab emtansine (Kadcyla®) or brentuximab vedotin (Adcetris®)). In some embodiments, the analyte is an antibody, such as a natural, synthetic, or recombinant antibody (or a portion thereof). In some embodiments, the analyte is an antibody formed in response to an allergen, a bacterial infection, or a viral infection.

As noted above, in some embodiments, the analyte is a monomeric protein. In some embodiments, the monomeric protein may have a first target region and a second target region that are different structures (e.g., epitopes) on the monomeric protein. In other words, the first target region and the second target region of the monomeric protein may be significantly different in structure. In other embodiments, the monomeric protein may have a first target region and a second target region that are substantially identical epitopes located at separate sites on the monomeric protein.

In some embodiments, the analyte is a multimeric protein complex. The multimeric protein complex may be a homomultimeric protein complex or a heteromultimeric protein complex. In some embodiments in which the analyte is a multimeric protein complex, the first targeting domain and the second targeting domain bind to adjacent proteins of the multimeric protein complex. Because the split reporter produces a signal only when the peptide fragments are in proximity to one another, the split reporter may be used to assess complex formation. Stated differently, split reporter systems that include both (1) a first peptide fragment that is attached to a first targeting domain that binds a first protein of a protein complex and (2) a second peptide fragment that is attached to a second targeting domain that binds to a second protein of a protein complex may be used to identify and/or quantify complex formation.

In some embodiments, the analyte is a soluble protein or biomolecule. For example, in some embodiments the analyte is a naturally occurring, endogenous, or xenobiotic protein or biomolecule. In some embodiments, the analyte is a growth factor, cytokine, hormone, a growth factor receptor, a cytokine receptor, or a hormone receptor. For example, the analyte may be selected from any of the following: adrenomedullin (AM), angiopoietin (Ang), autocrine motility factor, bone morphogenetic proteins (BMPs), ciliary neurotrophic factor family, ciliary neurotrophic factor (CNTF), leukemia inhibitory factor (LIF), interleukin-6 (IL-6), colony-stimulating factors, macrophage colony-stimulating factor (m-CSF), granulocyte colony-stimulating factor (G-CSF), granulocyte macrophage colony-stimulating factor (GM-CSF), epidermal growth factor (EGF), ephrins, ephrin A1, ephrin A2, ephrin A3, ephrin A4, ephrin A5, ephrin B1, ephrin B2, ephrin B3, erythropoietin (EPO), fibroblast growth factor (FGF) foetal bovine somatotrophin (FBS), a ligand from the GDNF family, glial cell line-derived neurotrophic factor (GDNF), neurturin, persephin, artemin, growth differentiation factor-9 (GDF9), hepatocyte growth factor (HGF), hepatoma-derived growth factor (HDGF), insulin, insulin-like growth factors, insulin-like growth factor-1 (IGF-1), insulin-like growth factor-2 (IGF-2), interleukins, IL-1, IL-2, IL-3, IL-4, IL-5, IL-6, IL-7, keratinocyte growth factor (KGF), migration-stimulating factor (MSF), macrophage-stimulating protein (also known as hepatocyte growth factor-like protein (HGFLP)), myostatin (GDF-8), neuregulins, neuregulin 1 (NRG1), neuregulin 2 (NRG2), neuregulin 3 (NRG3), neuregulin 4 (NRG4), neurotrophins, brain-derived neurotrophic factor (BDNF), nerve growth factor (NGF), neurotrophin-3 (NT-3), neurotrophin-4 (NT-4), placental growth factor (PGF), platelet-derived growth factor (PDGF), renalase (RNLS), T-cell growth factor (TCGF), thrombopoietin (TPO), transforming growth factors, transforming growth factor alpha (TGF-α), transforming growth factor beta (TGF-β), tumor necrosis factor (also known as tumor necrosis factor alpha (TNF-α)), vascular endothelial growth factor (VEGF), amylin (or islet amyloid polypeptide), anti-Müllerian hormone (or Müllerian-inhibiting factor or hormone), adiponectin, adrenocorticotropic hormone (or corticotropin), angiotensinogen, angiotensin, antidiuretic hormone (or vasopressin, arginine vasopressin), atrial-natriuretic peptide (or atriopeptin), brain natriuretic peptide, calcitonin, cholecystokinin, corticotropin-releasing hormone, cortistatin, enkephalin, endothelin, erythropoietin, follicle-stimulating hormone, galanin, gastric inhibitory polypeptide, gastrin, ghrelin, glucagon, glucagon-like peptide-1, gonadotropin-releasing hormone, growth hormone-releasing hormone, hepcidin, human chorionic gonadotropin, human placental lactogen, growth hormone, inhibin, insulin, insulin-like growth factor (or somatomedin), leptin, lipotropin, luteinizing hormone, melanocyte stimulating hormone, motilin, orexin, osteocalcin, oxytocin, pancreatic polypeptide, parathyroid hormone, pituitary adenylate cyclase-activating peptide, prolactin, prolactin releasing hormone, relaxin, renin, secretin, somatostatin, thrombopoietin, thyroid-stimulating hormone (or thyrotropin), thyrotropin-releasing hormone, vasoactive intestinal peptide, guanylin, uroguanylin, testosterone, dehydroepiandrosterone, androstenedione, dihydrotestosterone, aldosterone, estradiol, estrone, estriol, cortisol, progesterone, and calcitriol (1,25-dihydroxyvitamin D3).

In some embodiments, the analyte is a natural, synthetic, or recombinant immunoglobulin antibody (including a natural, synthetic, or recombinant antibody, or a portion thereof), an antibody fragment, or a derivative thereof. In some embodiments, the analyte is selected from any of the following: IgA, IgD, IgE, IgG, IgM, IgY, IgW, Vh, Vhh, a DARPin, a single-chain variable fragment (scFv), a monobody, a diabody, or a portion thereof.

In some embodiments, the analyte includes or consists of DNA or RNA, such as a polynucleotide formed from DNA, RNA, or a combination of DNA and RNA.

In some embodiments, the analyte is a biological product or biosimilar. In some embodiments, the analyte is selected from any of the following: abatacept, abciximab, abobotulinumtoxinA, adalimumab, adalimumab-atto, ado-trastuzumab emtansine, aflibercept, agalsidase beta, albiglutide, aldesleukin, alemtuzumab, alglucosidase alfa, alglucosidase alfa, alirocumab, alteplase, cathflo activase, anakinra, asfotase alfa, asparaginase, asparaginase Erwinia chrysanthemi, atezolizumab, basiliximab, becaplermin, belatacept, belimumab, bevacizumab, bezlotoxumab, blinatumomab, brentuximab vedotin, canakinumab, capromab pendetide, certolizumab pegol, cetuximab, collagenase, collagenase clostridium histolyticum, daclizumab, daclizumab, daratumumab, darbepoetin alfa, denileukin diftitox, denosumab, dinutuximab, dornase alfa, dulaglutide, ecallantide, eculizumab, elosulfase alfa, elotuzumab, Empliciti, epoetin alfa, etanercept, etanercept-szzs, evolocumab, filgrastim, filgrastim-sndz, galsulfase, glucarpidase, golimumab, ibritumomab tiuxetan, idarucizumab, idursulfase, incobotulinumtoxinA, infliximab, infliximab-dyyb, interferon alfa-2b, interferon alfa-n3, interferon beta-1a, interferon beta-1b, interferon beta-1, interferon gamma-1b, ipilimumab, ixekizumab, laronidase, mepolizumab, methoxy polyethylene glycol-epoetin beta, metreleptin, natalizumab, necitumumab, nivolumab, obiltoxaximab, obinutuzumab, ocriplasmin, ofatumumab, olaratumab, omalizumab, onabotulinumtoxinA, oprelvekin, palifermin, palivizumab, panitumumab, parathyroid hormone, pegaspargase, pegfilgrastim, peginterferon alfa-2a, peginterferon alfa-2b, peginterferon beta-1a, pegloticase, pembrolizumab, pertuzumab, ramucirumab, ranibizumab, rasburicase, raxibacumab, reslizumab, reteplase, rilonacept, rimabotulinumtoxinB, rituximab, romiplostim, sargramostim, sebelipase alfa, secukinumab, siltuximab, tbo-filgrastim, tenecteplase, tocilizumab, tocilizumab, trastuzumab, ustekinumab, vedolizumab, or ziv-aflibercept.

In some embodiments, the analyte is a therapeutic monoclonal antibody. For example, in some embodiments, the analyte is selected from any of the following monoclonal antibodies: abciximab (e.g., ReoPro), a chimeric antibody that targets and inhibits glycoprotein Ilb/Illa, and is used for the treatment of cardiovascular disease; adalimumab (e.g., Humira), a human antibody that inhibits tumor necrosis factor alpha (TNF-α) signaling, and is used for the treatment of rheumatoid arthritis, psoriatic arthritis, ankylosing spondylitis, Crohn's disease, ulcerative colitis, chronic psoriasis, hidradenitis suppurativa, and juvenile idiopathic arthritis; alemtuzumab (e.g., Campath), a humanized antibody that targets CD52, and is used for the treatment of chronic lymphocytic leukemia; basiliximab (e.g., Simulect), a chimeric antibody that targets the interleukin-2 receptor alpha chain (CD25), and is used for the treatment of transplant rejection; belimumab (e.g., Benlysta), a human antibody that targets and inhibits B-cell activating factor, and is used for the treatment of systemic lupus erythematosus; bevacizumab (e.g., Avastin), a humanized antibody that targets vascular endothelial growth factor (VEGF), and is used for the treatment of colorectal cancer, certain lung cancers, renal cancers, ovarian cancers, glioblastoma multiforme of the brain, and age related macular degeneration (off-label); brentuximab vedotin (e.g., Adcetris), a chimeric antibody that targets CD30, and is used for the treatment of anaplastic large cell lymphoma (ALCL) and Hodgkin lymphoma; canakinumab (e.g., llaris), a human antibody that targets interleukin 1β (IL-1β), and is used for the treatment of cryopyrin-associated periodic syndrome (CAPS); cetuximab (e.g., Erbitux), a chimeric antibody that targets epidermal growth factor receptor (EGFR), and is used for the treatment of colorectal cancer and head and neck cancer; certolizumab pegol (e.g., Cimzia), a humanized antibody that targets and inhibits TNF-α signaling, and is used for the treatment of Crohn's disease; daclizumab (e.g., Zenapax), a humanized antibody that targets interleukin-2 receptor alpha chain (CD25), and is used for the treatment of transplant rejection; daratumumab (e.g., Darzalex), a human antibody that targets CD38, and is used for the treatment of multiple myeloma; denosumab (e.g., Prolia and Xgeva), a human antibody that targets RANK ligand inhibitor, and is used for the treatment of postmenopausal osteoporosis and solid tumor bony metasteses; eculizumab (e.g., Soliris), a humanized antibody that targets complement system protein C5, and is used for the treatment of paroxysmal nocturnal hemoglobinuria; efalizumab (e.g., Raptiva), a humanized antibody that targets CD11a, and is used for the treatment of psoriasis; golimumab (e.g., Simponi), a human antibody that targets TNF-α inhibitor, and is used for the treatment of rheumatoid arthritis, psoriatic arthritis and ankylosing spondylitis; ibritumomab tiuxetan (e.g., Zevalin), a murine antibody that targets CD20, and is used for the treatment of non-Hodgkin lymphoma (in combination with yttrium-90 or indium-111); infliximab (e.g., Remicade), a chimeric antibody that targets and inhibits TNF-α signaling, and is used for the treatment of Crohn's disease, ulcerative colitis, psoriasis, psoriatic arthritis, ankylosing spondylitis and rheumatoid arthritis; ipilimumab (MDX-101) (e.g., Yervoy), a human antibody that targets and blocks CTLA-4, and is used for the treatment of melanoma; muromonab-CD3 (e.g., Orthoclone OKT3), a murine antibody that targets T cell CD3 receptor, and is used for the treatment of transplant rejection; natalizumab (e.g., Tysabri), a humanized antibody that targets alpha-4 integrin, and is used for the treatment of multiple sclerosis and Crohn's disease; nivolumab (e.g., Opdivo), a human antibody that targets and blocks programmed cell death protein 1 (PD-1), and is used for the treatment of melanoma and squamous-cell carcinoma of the lung; ofatumumab (e.g., Arzerra), a human antibody that targets CD20, and is used for the treatment of chronic lymphocytic leukemia; omalizumab (e.g., Xolair), a humanized antibody that targets immunoglobulin E (IgE), and is used for the treatment of mainly allergy-related asthma; palivizumab (e.g., Synagis), a humanized antibody that targets an epitope of the RSV F-protein, and is used for the treatment of respiratory syncytial virus; panitumumab (e.g., Vectibix), a human antibody that targets epidermal growth factor receptor (EGFR), and is used for the treatment of colorectal cancer; pembrolizumab (e.g., Keytruda), a humanized antibody that targets the programmed cell death 1 (PD-1) receptor, and is used for the treatment of melanoma and non-small cell lung cancer; ranibizumab (e.g., Lucentis), a humanized antibody that targets vascular endothelial growth factor A (VEGF-A), and is used for the treatment of macular degeneration; rituximab (e.g., Rituxan and Mabthera), a chimeric antibody that targets CD20, and is used for the treatment of non-Hodgkin lymphoma; tocilizumab or atlizumab (e.g., Actemra or RoActemra), a humanized antibody that targets interleukin 6 receptor (IL-6R), and is used for the treatment of rheumatoid arthritis and systemic juvenile idiopathic arthritis; tositumomab (e.g., Bexxar), a murine antibody that targets CD20, and is used for the treatment of non-Hodgkin lymphoma; trastuzumab (e.g., Herceptin), a humanized antibody that targets receptor tyrosine-protein kinase erbB-2 (ErbB2) or CD340, and is used for the treatment of breast cancer; ustekinumab (e.g., Stelara), a human antibody that targets interleukin 12 (IL-12) and interleukin 23 (IL-23), and is used for the treatment of psoriatic arthritis and plaque psoriasis; and vedolizumab (e.g., Entyvio), a humanized antibody that targets integrin a437, and is used for the treatment of Crohn's disease and ulcerative colitis.

In some embodiments, the analyte may be a cell surface protein or biomolecule, or be bound to a membrane of a cell. For example, in some embodiments, the analyte is a cell-surface marker and/or a cell-surface receptor. In some embodiments, the analyte is selected from any of the following: adrenergic receptor, olfactory receptors, receptor tyrosine kinases, epidermal growth factor receptor, insulin receptor, fibroblast growth factor receptors, high affinity neurotrophin receptors, ephrin receptors, integrins, low affinity nerve growth factor receptor, NMDA receptor, or immune receptors. In some embodiments, the analyte is HER2, a portion of HER2, or a multimeric protein complex that includes HER2. In some embodiments, the detected HER2 is obtained from serum.

In some embodiments, the analyte is a viral or bacterial protein, DNA, or RNA. Stated differently, in some embodiments, the analyte is DNA, RNA, or a protein from a virus or a bacterium, such as a pathogenic virus or bacterium. Exemplary viruses and bacteria for the analyte can be selected from any of the following: astrovirus, chickenpox, dengue virus, Ebola, foot-and-mouth disease virus, hepatitis A, hepatitis B, hepatitis C, herpes, human immunodeficiency virus, human papillomavirus, influenza, Japanese encephalitis, measles, mumps, Naples virus, parvovirus, rabies, rubella, shingles, smallpox, Toscana virus, Varicella Zoster virus, West Nile virus, yellow fever, Zika virus, Bacillus anthracis, Bacillus cereus, Bartonella henselae, Bartonella quintana, Bordetella pertussis, Borrelia burgdorferi, Borrelia garinii, Borrelia afzelii, Borrelia recurrentis, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Enterococcus faecalis, Enterococcus faecium, Escherichia coli, Francisella tularensis, Haemophilus influenzae, Helicobacter pylori, Legionella pneumophila, Leptospira interrogans, Leptospira santarosai, Leptospira weilii, Leptospira noguchii, Listeria monocytogenes, Mycobacterium leprae, Mycobacterium tuberculosis, Mycobacterium ulcerans, Mycoplasma pneumoniae, Neisseria gonorrhoeae, Neisseria meningitidis, Pseudomonas aeruginosa, Rickettsia rickettsii, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Ureaplasma urealyticum, Vibrio cholerae, Yersinia pestis, Yersinia enterocolitica, and Yersinia pseudotuberculosis.

In some embodiments, the analyte is a complex formed from protein interaction partners. Any suitable set of protein interaction partners may be used as an analyte. In some embodiments, an analyte may be a cell surface or membrane-bound complex, such as the EGFR-HER2 complex, the HER2-HER2 homodimer, or the HER2-HER3 heterodimer. In other embodiments, the analyte is a soluble complex, such as the P53-MDM2 complex, VEGF dimer, BAK-BclxL complex, or the XIAP-Smac complex.

As noted above, in some embodiments, the analyte is a modified protein. The modification to the protein may be any suitable modification (e.g., a post-translation modification). In some embodiments, the modification is a naturally occurring modification. In some embodiments, the modification is indicative of a normal state or a disease state. In some embodiments, the modification is a synthetic modification, such as a protein that has been modified to improve pharmacokinetics, improve efficacy, and/or to reduce toxicity. Exemplary post-translational modifications include phosphorylation, glycosylation, lipidation, or acylation. In some embodiments, the modified protein is a pegylated protein, such as, for example, pegadamase, pegaspargase, peginterferon-2b, peginterferon-2a, pegfilgrastim, pegvisomant, pegaptanib, mPEG-epoetin, certolizumab, PEG-uricase. In some embodiments, the protein is modified after removal from a cellular environment. Such modification can improve pharmacokinetics, enhance efficacy, and/or decrease toxicity of the modified protein. In some embodiments, the analyte is an antibody-drug conjugate. In some embodiments, the analyte is an antibody-enzyme conjugate. In some embodiments, the analyte is a fusion protein.

In some embodiments, the analyte is an autoantibody. In such embodiments, the analyte may be an autoantibody that recognizes and binds a self-antigen. In some embodiments, the analyte is selected from any of the following: antinuclear antibodies including anti-SSA/Ro autoantibodies, anti-La/SS-B autoantibodies, anti-centromere antibodies, anti-double-stranded DNA (dsDNA) antibodies, anti-Jo1 antibodies or anti-histidine-tRNA ligase antibodies, anti-ribonucleoprotein (RNP) antibodies, anti-snRNP core proteins antibodies or anti-Smith antibodies, anti-topoisomerase antibodies, anti-histone antibodies, anti-nucleoporin 62 antibodies or anti-p62 antibodies, anti-sp 100 or anti-sp 100 nuclear antigen antibodies, anti-nucleoporin 210 kDA antibodies or anti-glycoporin-210 antibodies, anti-transglutaminase antibodies including anti-tTG antibodies and anti-eTG antibodies, anti-ganglioside antibodies including anti-ganglioside GQ1B antibodies, anti-ganglioside GD3 antibodies, and anti-ganglioside GM1 antibodies, anti-actin antibodies, anti-cyclic citrulllinated peptide antibodies, anti-liver kidney microsomal type 1 antibodies, anti-lupus anticoagulant antibodies, anti-thrombin antibodies, anti-phospholipid antibodies, anti-neutrophil antibodies including anti-c-ANCA antibodies and anti-p-ANCA antibodies, anti-rheumatoid factor antibodies, anti-smooth muscle antibodies, anti-mitochondrial antibodies, anti-signal recognition particle (SRP) antibodies, anti-nicotinic acetylcholine receptor (AChR) antibodies, anti-muscle-specific kinase (MUSK) antibodies, anti-voltage-gated calcium channel (VGCC) antibodies including anti-P/Q-type voltage-gated calcium channel antibodies, anti-thyroid antibodies including anti-thyroid peroxidase (TPO) antibodies, anti-thyroglobulin antibodies (TgAbs), and anti-thyrotropin receptor antibodies (TRAbs), anti-Hu (ANNA-1) antibodies, anti-Yo antibodies, anti-Ma antibodies, anti-Ri (ANNA-2) antibodies, anti-Tr antibodies, anti-amphyiphysin antibodies, anti-glutamate decarboxylase (GAD) antibodies, anti-voltage-gated potassium channel (VGKC) antibodies, anti-collapsin response mediator protein 5 (CRMP-5) antibodies, anti-N-methyl-D-aspartate receptor (NMDAr) antibodies, and anti-aquaporin or anti-NMO antibodies. In some embodiments, the autoantibody binds to an acetylcholine receptor (e.g., a nicotinic acetylcholine receptor).

In some embodiments, when a particular autoantibody is known to interact with a particular self-antigen target to cause an autoimmune disease or disorder, the disclosed methods may be used to diagnose or confirm that a patient has, or is at risk of developing, a particular form of autoimmune disease or autoimmune-associated disease.

For example, when a patient is suspected of having systemic lupus erythematosus (SLE), the underlying cause of that SLE may be confirmed and/or attributed to the presence of anti-SSA/Ro autoantibodies, anti-double-stranded DNA (dsDNA) antibodies, anti-histone antibodies, anti-snRNP core proteins antibodies or anti-Smith antibodies and/or anti-lupus anticoagulant antibodies or anti-thrombin autoantibodies by the methods described herein.

When a patient is suspected of having neonatal heart block, the underlying cause may be confirmed and/or attributed to the presence of anti-SSA/Ro autoantibodies by the methods described herein.

When a patient is suspected of having primary Sjögren's syndrome the underlying cause of the syndrome may be confirmed and/or attributed to the presence of anti-SSA/Ro autoantibodies or anti-La/SS-B autoantibodies by the methods described herein.

When a patient is suspected of having CREST syndrome the underlying cause of the syndrome may be confirmed and/or attributed to the presence of anti-centromere antibodies by the methods described herein.

When a patient is suspected of having inflammatory myopathy, the underlying cause may be confirmed and/or attributed to the presence of anti-Jo1 antibodies or anti-histidine-tRNA ligase autoantibodies by the methods described herein.

When a patient is suspected of having mixed connective tissue disease, the underlying cause may be confirmed and/or attributed to the presence of anti-ribonucleoprotein or anti-RNP autoantibodies by the methods described herein.

When a patient is suspected of having systemic sclerosis, the underlying cause of the disease may be confirmed and/or attributed to the presence of anti-Scl-70 antibodies or anti-type I topoisomerase antibodies by the methods described herein.

When a patient is suspected of having primary biliary cirrhosis, the underlying cause of the disease may be confirmed and/or attributed to the presence of anti-nucleoporin 62 antibodies or anti-p62 antibodies, anti-sp100 or anti-sp100 nuclear antigen antibodies, anti-nucleoporin 210 kDA antibodies or anti-glycoporin-210 antibodies by the methods described herein.

When a patient is suspected of having celiac disease, the underlying cause of the disease may be confirmed and/or attributed to the presence of anti-transglutaminase (tTG) antibodies by the methods described herein.

When a patient is suspected of having dermatitis herpetiformis, the underlying cause of the disease may be confirmed and/or attributed to the presence of anti-transglutaminase (eTG) antibodies by the methods described herein.

When a patient is suspected of having Miller-Fisher syndrome the underlying cause of the syndrome may be confirmed and/or attributed to the presence of anti-ganglioside GQ1B antibodies by the methods described herein.

When a patient is suspected of having acute motor axonal neuropathy (AMAN) the underlying cause of the disease may be confirmed and/or attributed to the presence of anti-ganglioside GD3 antibodies by the methods described herein.

When a patient is suspected of having multifocal motor neuropathy with conduction block (MMN) the underlying cause of the disease may be confirmed and/or attributed to the presence of anti-ganglioside GM1 antibodies by the methods described herein.

When a patient is suspected of having rheumatoid arthritis the underlying cause of the disease may be confirmed and/or attributed to the presence of anti-cyclic citrullinated peptide (CCP), or anti-rheumatoid factor antibodies by the methods described herein.

When a patient is suspected of having autoimmune hepatitis, or chronic autoimmune hepatitis, the underlying cause of the disease may be confirmed and/or attributed to the presence of anti-liver kidney microsomal type 1 antibodies or anti-smooth muscle antibodies by the methods described herein.

When a patient is suspected of having antiphospholipid syndrome the underlying cause of the syndrome may be confirmed and/or attributed to the presence of anti-phospholipid antibodies by the methods described herein.

When a patient is suspected of having granulomatosis with polyangiitis the underlying cause of the disease may be confirmed and/or attributed to the presence of anti-neutrophil cytoplasmic (c-ANCA) antibodies by the methods described herein.

When a patient is suspected of having microscopic polyangiitis, eosinophilic granulaomatosis with polyangiitis, or systemic vasculitides, the underlying cause of the disease may be confirmed and/or attributed to the presence of anti-neutrophil perinuclear (p-ANCA) antibodies by the methods described herein.

When a patient is suspected of having primary biliary cirrhosis the underlying cause may be confirmed and/or attributed to the presence of anti-mitochondrial antibodies by the methods described herein.

When a patient is suspected of having polymyositis the underlying cause may be confirmed and/or attributed to the presence of anti-signal recognition particle (SRP) antibodies by the methods described herein.

When a patient is suspected of having scleromyositis the underlying cause may be confirmed and/or attributed to the presence of anti-exosome complex antibodies by the methods described herein.

When a patient is suspected of having myasthenia gravis the underlying cause may be confirmed and/or attributed to the presence of anti-acetylcholine receptor (anti-AChr), or anti-muscle-specific kinase (MUSK) antibodies by the methods described herein.

When a patient is suspected of having Lambert-Eaton myasthenic syndrome the underlying cause of the syndrome may be confirmed and/or attributed to the presence of anti-voltage-gated calcium channel (P/Q-type) (VGCC) antibodies by the methods described herein.

When a patient is suspected of having Hashimoto's thyroiditis or Graves' disease the underlying cause may be confirmed and/or attributed to the presence of anti-thyroid peroxidase (TPO) antibodies, anti-thyroglobulin antibodies (TgAbs), or anti-thyrotropin receptor antibodies (TRAbs) by the methods described herein.

When a patient is suspected of having paraneoplastic cerebellar degeneration, limbic encephalitis, encephalomyelitis, subacute sensory neuronopathy, or choreathetosis, the underlying cause of the disease may be confirmed and/or attributed to the presence of anti-Hu (ANNA-1) autoantibodies, anti-Yo autoantibodies, or anti-amphiphysin antibodies by the methods described herein.

When a patient is suspected of having opsoclonus myoclonus syndrome the underlying cause of the syndrome may be confirmed and/or attributed to the presence of anti-Ri (ANNA-2) antibodies by the methods described herein.

When a patient is suspected of having paraneoplastic cerebellar syndrome the underlying cause of the syndrome may be confirmed and/or attributed to the presence of anti-Tr or anti-glutamate receptor autoantibodies by the methods described herein.

When a patient is suspected of having stiff person syndrome the underlying cause of the syndrome may be confirmed and/or attributed to the presence of anti-amphiphysin or anti-glutamate decarboxylase (anti-GAD) autoantibodies by the methods described herein.

When a patient is suspected of having Isaac's syndrome (autoimmune neuromyotonia) or limbic encephalitis the underlying cause may be confirmed and/or attributed to the presence of anti-voltage-gated potassium channel (anti-VGKC) autoantibodies by the methods described herein.

When a patient is suspected of having optic neuropathy or chorea the underlying cause may be confirmed and/or attributed to the presence of anti-collapsin response mediator protein 5 (anti-CRMP-5) autoantibodies by the methods described herein.

When a patient is suspected of having Sydenham's chorea or pediatric autoimmune neuropsychiatric disease associated with Streptococcus (PANDAS), the underlying cause may be confirmed and/or attributed to the presence of anti-basal ganglial neuron autoantibodies by the methods described herein.

When a patient is suspected of having anti-N-methyl-D-aspartate (NDMA) receptor encephalitis the underlying cause may be confirmed and/or attributed to the presence of anti-NDMAr autoantibodies by the methods described herein.

When a patient is suspected of having neuromyelitis optica (Devic's syndrome) the underlying cause may be confirmed and/or attributed to the presence of anti-aquaporin-4 autoantibodies or NMO antibodies by the methods described herein.

The specific examples described above to confirm the underlying cause of a given autoimmune disease or autoimmune-related disease are only exemplary. Indeed, the disclosed methods may be used to diagnose a wide range of known autoimmune or autoimmune-related diseases, including: Acute Disseminated Encephalomyelitis (ADEM), Acute necrotizing hemorrhagic c leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenia purpura (ATP), Autoimmune thyroid disease, Autoimmune urticarial, Axonal & neuronal neuropathies, Balo disease, Behcet's disease, Bullous pemphigoid, Cardiomyopathy, Castleman disease, Celiac disease, Chagas disease, Chronic fatigue syndrome-associated autoimmune diseases, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibromyalgia-associated autoimmune diseases, Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenia purpura (ITP), IgA nephropathy, IgG4-related sclerosing disease, Immunoregulatory lipoproteins, Inclusion body myositis, Interstitial cystitis, Juvenile arthritis, Juvenile diabetes (Type 1 diabetes), Juvenile myositis, Kawasaki syndrome, Lambert-Eaton syndrome, Leukocytoclastic vasculitis, Lichen planus, Lichen sclerosus, Ligneous conjunctivitis, Linear IgA disease (LAD), Lupus (SLE), Lyme disease (chronic), Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatic, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenia purpura (TTP), Tolosa-Hunt syndrome, Transverse myelitis, Type 1 diabetes, Ulcerative colitis, Undifferentiated connective tissue disease (UCTD), Uveitis, Vasculitis, Vesiculobullous dermatosis, Vitiligo, and Wegener's granulomatosis (now termed Granulomatosis with Polyangiitis (GPA)).

In some embodiments, the affinity of the first and/or second targeting domains to the corresponding target regions may be greater than the affinity of the peptide fragments of the split protein to the remaining peptide fragment(s) of the split protein, thereby reducing background signal. In some embodiments the affinity of the first and/or second targeting domains to the corresponding target regions may be at least 1, 2, 3, and/or 4 orders of magnitude greater than the affinity of a peptide fragment of the split protein to the remaining peptide fragment(s) of the split protein complex. In some embodiments, the first targeting domain and the second targeting domain do not bind directly to each other. In other words, the first targeting domain and the second targeting domain may be brought near each other not through any direct interaction between the first targeting domain and the second targeting domain but through binding to the same analyte.

Related kits may be used to detect an analyte. In some embodiments, the kit includes a first vector. The first vector may include a first sequence that encodes a first peptide fragment of a tripartite split enzyme. The vector may be configured to facilitate insertion of a nucleotide sequence for a first targeting domain such that expression of the resulting vector yields a fusion protein that includes both the first targeting domain and the first peptide fragment.

In some embodiments, the kit may additionally or alternatively include a second vector. The second vector may include a second sequence that encodes a second peptide fragment of a tripartite split enzyme. The vector may be configured to facilitate insertion of a nucleotide sequence for a second targeting domain such that expression of the resulting vector yields a fusion protein that includes both the second targeting domain and the second peptide fragment.

In some embodiments, the kit may include a third fragment of the tripartite split enzyme. The third peptide fragment may have a mass of between 16 kDa and 17 kDa. In some embodiments, the third peptide fragment has at least 70%, 80%, 90%, 95%, or 100% sequence identity to the sequence of SEQ ID NO 3. In some embodiments, the third peptide fragment is between 140 and 150 amino acids. For example, in some embodiments, the third peptide is between 145 and 150 amino acids (e.g., 148 amino acids). In some embodiments, the kit may additionally or alternatively include a vector for expressing the third fragment of the tripartite split enzyme.

In other embodiments, a kit may include a first agent, a second agent, and a third agent, as defined above. The first agent may include a first targeting domain and a first peptide fragment of a split reporter protein. The second agent may include a second targeting domain and a second peptide fragment of the split reporter protein. The third agent may include a third fragment of the split reporter protein. In some such embodiments, the first and second targeting domains determine the antigen to be detected using the kit. Such a kit may optionally include the reagents necessary to generate a detectable signal when the first, second and third fragments of the split reporter protein are assembled into a functional reporter protein, such as a functional enzyme reporter. In such embodiments, the kit may optionally include the substrate for the assembled functional enzyme reporter, and instructions for use of the kit for detecting a specific antigen. In some embodiments of such kits, the specific analyte to be detected may be selected from any of the analytes disclosed herein. In some embodiments of such kits, the specific analyte to be detected may be selected from an antibody formed in response to an allergen, a bacterial infection, or a viral infection, a therapeutic antibody, or and autoantibody that binds a self-antigen.

Some embodiments within the scope of this disclosure may be detection reagents. Some detection reagents may include a targeting domain and a peptide fragment of a tripartite split reporter protein. The peptide fragment of the detection reagent may have a mass of less than 3 kDa. In some embodiments, the peptide fragment is 15, 14, 13, 12, 11, or 10 amino acids in length. In some embodiments, the peptide fragment has at least 80%, 90%, 95%, and/or 100% sequence identity to the sequence of SEQ ID NO 1. In other embodiments, the peptide fragment has at least 80%, 90%, 95%, and/or 100% sequence identity to the sequence of SEQ ID NO 2. The peptide fragment of a detection reagent may be capable of forming a complex with one or more other peptide fragments to form an intact split reporter protein.

Some embodiments may be nucleic acids. For example, some nucleic acids may include a sequence that includes a promoter, an insertion site, and a region that encodes a peptide fragment of a split reporter enzyme. The nucleic acid may be configured such that insertion of a sequence that encodes a targeting domain at the insertion site allows for expression of a fusion protein that includes the targeting domain and the peptide fragment. Stated differently, the sequence of the resulting nucleotide, which encodes a fusion protein, may be operably linked to a promoter.

Some embodiments include methods for detecting an analyte in a mixture (e.g., a mixture in a vessel). The method may include the step of delivering a plurality of agents into a mixture, wherein each of the agents includes a portion of a split reporter protein. For example, some methods may include the steps of delivering a first agent into the mixture, and delivering a second agent into the mixture. The first agent may include a first targeting domain and a first peptide fragment of the split reporter protein. The second agent may include a second targeting domain and a second peptide fragment of the split reporter protein. In some embodiments, the split reporter protein is a binary split reporter protein (i.e., the split reporter protein has only two fragments). In other embodiments (e.g., where the split reporter protein is a ternary split reporter protein), the method may further include the step of delivering a third agent into the mixture. The third agent may include or consist essentially of a third fragment of the split reporter protein. In some embodiments, the first agent and the second agent are delivered in substantially equimolar amounts (e.g., a ratio of 1:1 (+0.2)). In some embodiments, the first agent and the second agent are not delivered in substantially equimolar amounts. In some embodiments, the first agent and the second agent are delivered as purified proteins.

Upon delivery into the mixture, the first targeting domain of the first agent may bind to a first target region of the analyte. Similarly, the targeting domain of the second agent may bind to a second target region of the analyte. Such binding may increase the local concentration of these components. In embodiments that involve a binary split reporter protein, the first agent and the second agent may bind to one another to form an active complex. In embodiments that involve a ternary split reporter protein, the first agent and the second agent may bind (e.g., non-covalently) to a third agent to form an active complex. In both cases, the active complex is formed essentially only where the first agent and the second agent are in close proximity. These methods, in which binding of the individual peptide fragments to form an active complex is driven by the binding of targeting domains to an analyte, may be termed “target-binding activated complementation” or “target-engaged complementation.”

In some embodiments, the first agent and the second agent are delivered to the mixture prior to delivery of the third agent. In other embodiments, the first agent, the second agent, and the third agent are simultaneously delivered to the mixture. Numerous different ordering of steps is possible, as will be understood by a skilled artisan having the benefit of this disclosure.

In some embodiments, the method further includes the step of delivering a substrate of the active complex into the mixture. Some methods may include the step of detecting light emitted from the mixture after the first agent, the second agent, and the third agent have been delivered to the mixture. The amount of light emitted from the mixture may be proportional to the amount of analyte that is contained therein. In some embodiments, the method is capable of detecting the analyte at concentrations of less than 5 pM, such as concentrations of less than 1 pM.

In some embodiments, the method is practiced outside of living cells. Stated differently, in some embodiments, the analyte to be detected is not disposed within a cell. In some embodiments, the analyte is detected in or from a cell lysate. In some embodiments, the method is carried out in a biological fluid. For example, some methods may be carried out in human serum or in a mixture that includes human serum. Other methods may be carried out in saliva or in a mixture that includes saliva. Other methods may be carried out in urine or in a mixture that includes urine. Detection of one or more analytes from other biological fluids is also contemplated.

In some embodiments, the method for detecting the analyte does not include a blocking step. Stated differently, in some embodiments, the method does not require delivery of a blocking agent, such as bovine serum albumin or milk proteins.

In some embodiments, the method for detecting the analyte does not include a wash step. Stated differently, in some embodiments, an unbound first agent and second agent need not be removed from the mixture prior to detection.

In some embodiments, the method for detecting the analyte does not involve immobilization of the analyte. In other words, in some embodiments, the analyte may be in solution (e.g., a homogeneous solution) when detected. In other embodiments, the analyte is immobilized onto a surface. In other embodiments, the analyte is present on the surface of a tissue or tissue sample (e.g., a prepared tissue sample mounted on a microscope slide).

In some embodiments, the method does not require the use of protein disulfide isomerase. Stated differently, in some methods, none of the first peptide fragment, the second peptide fragment, or the third peptide fragment contacts a protein disulfide isomerase.

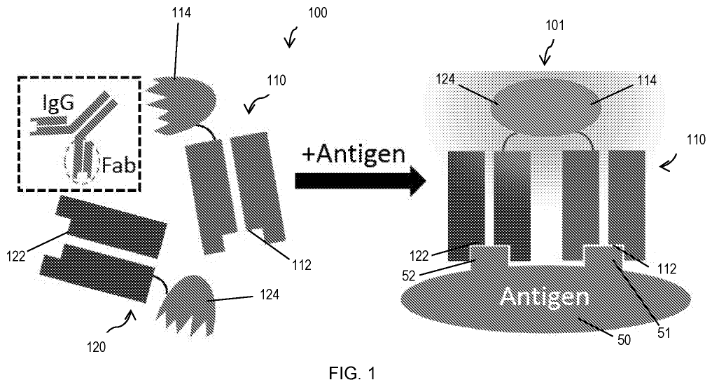

FIG. 1 shows an embodiment of a split reporter system 100. The split reporter system 100 includes a first agent 110 and a second agent 120. The first agent 110 includes a first targeting domain 112 and a first peptide fragment 114 of a split reporter protein 101. The second agent 120 includes a second targeting domain 122 and a second peptide fragment 124 of the split reporter protein 101. Each targeting domain 112, 122 has an affinity to a particular target region 51, 52 of an analyte 50. In the depicted embodiment, the targeting domains are Fabs and the analyte 50 is a corresponding bivalent antigen.

When the first agent 110 and the second agent 120 are mixed with the antigen 50, the first targeting domain 112 of the first agent binds to a first epitope 51 of the antigen 50 and the second targeting domain 122 of the second agent 120 binds to the second epitope 52 of the antigen 50, thereby bringing the first peptide fragment 114 and the second peptide fragment 124 into close proximity. The close proximity of the first peptide fragment 114 to the second peptide fragment 124 leads to formation of an active reporter protein complex (i.e., intact split reporter protein 101). The active reporter protein complex can produce a detectable signal (e.g., fluorescence of luminescence). Thus, the signal produced by the active reporter protein complex may be used to detect the presence (and/or determine the quantity) of analyte 50 in a mixture.

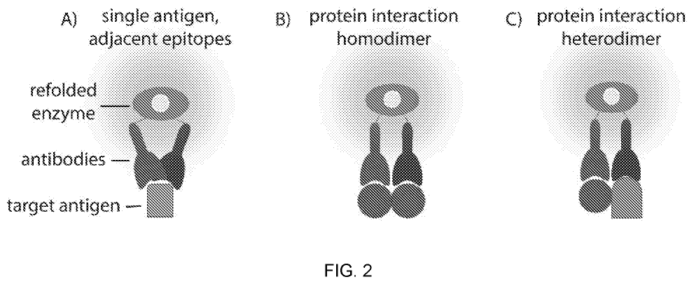

FIG. 2 is a schematic showing various targeting modes (FIGS. 2A-2C) for activating fragment complementation in a split reporter system. For example, FIG. 2A shows binding of targeting domains (e.g., antibodies) to adjacent target regions (e.g., epitopes) on a single bivalent analyte (e.g., antigen). FIG. 2B shows binding of targeting domains to identical target regions on separate proteins of a homomultimeric protein complex. And FIG. 2C shows binding of targeting domains to different proteins of a heteromultimeric protein complex. Any of the targeting modes shown in FIG. 2 may be used to detect an analyte.

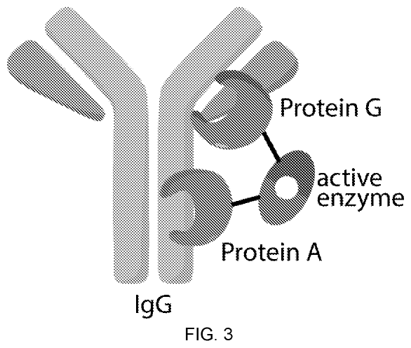

FIG. 3 is a schematic showing a fragment complementation system for detecting an antibody (e.g., IgG). In the depicted embodiment, a first agent includes (1) a first peptide fragment of a split reporter protein and (2) a first targeting domain (e.g., protein A) with affinity to a first target region of the antibody. The second agent includes (1) a second peptide fragment of a split reporter protein and (2) a second targeting domain (e.g., protein G) with affinity to a second target region of the antibody. When the first agent and the second agent are placed in a mixture that includes the antibody, the first agent and the second agent are drawn into proximity, which leads to formation of the split reporter protein complex.

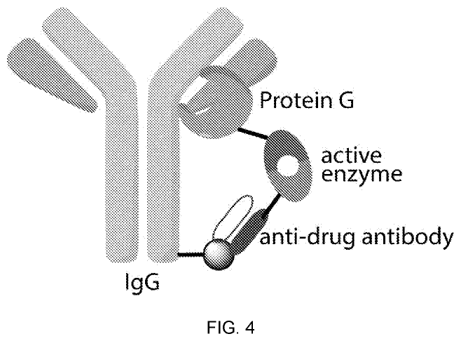

FIG. 4 is a schematic showing a fragment complementation system for detecting an antibody-drug conjugate. Such conjugates may be used, for example, to direct a drug to a particular location within a patient's body. In the depicted embodiment, the first agent includes a targeting domain with affinity to a drug that is attached to the antibody (e.g., via a linker), and the second agent includes a targeting domain (e.g., protein G) with affinity to a target region of the antibody. Such a fragment complementation system can be used to detect or quantify an antibody-drug conjugate in a mixture.

While the embodiments shown in FIGS. 1-4 are shown as two-component fragment complementation systems, a skilled artisan with the benefit of this disclosure will understand that analogous three-component fragment complementation systems are also within the scope of this disclosure.

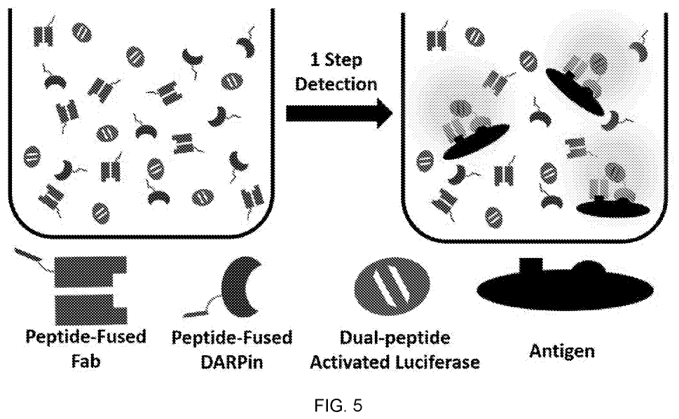

FIG. 5 is a schematic showing detection of a single antigen via a tripartite fragment complementation system. More particularly, FIG. 5 shows a first agent that includes a first targeting domain (e.g., a Fab) and a first peptide fragment of a split reporter protein. The second agent includes a second targeting domain (e.g., a DARPin) and a second peptide fragment of a split reporter system. The embodiment shown in FIG. 5 also includes a third peptide fragment (i.e., a dual-peptide activated luciferase) of a split reporter system. When mixed in the presence of a bivalent antigen, the first agent and the second agent bind to different epitopes on the same bivalent antigen through their respective targeting domains. The first peptide fragment and the second peptide fragment may bind to the third peptide fragment to form an active split reporter protein complex (e.g., an active luciferase). In embodiments where the affinity of the first targeting domain and the second targeting domain to the antigen is significantly higher than the affinity of the components of the tripartite complex to each other, a signal generated from the activated complex (e.g., luminescence) may indicate the presence (or quantity) of antigen in a mixture. In some embodiments, one-step detection is possible. For example, an antigen may be added to a mixture that includes the three components of the tripartite fragment complementation system. The increase in signal from the activated complex may be used to detect and/or quantify the analyte.

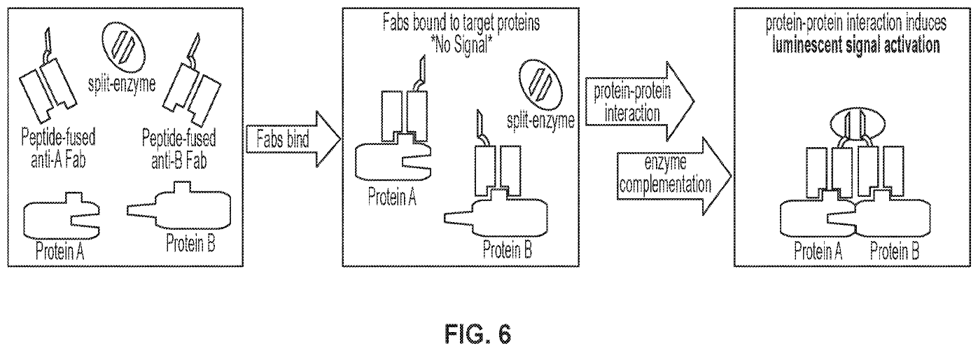

FIG. 6 is a schematic that shows one embodiment for using a tripartite fragment complementation system to detect a protein-protein interaction. As shown in FIG. 6, the components of the fragment complementation system can be mixed with a pair of protein interaction partners. One agent of the fragment complementation system may bind to the first protein while a second agent of the fragment complementation system binds to a second protein. Upon (1) binding of the first protein to the second protein and (2) complementation with a third component of the fragment complementation system, an activated complex is formed, thereby allowing for detection of the interaction between the two proteins. In some embodiments, analogous detection can be accomplished where two proteins do not directly interact, but are in close proximity to one another.

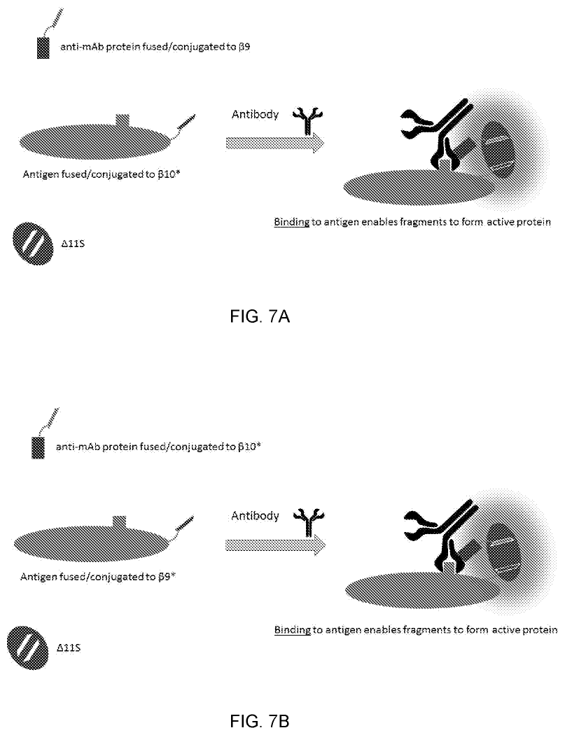

FIGS. 7A and 7B show alternative strategies for detection of an antibody (e.g., a therapeutic antibody or an autoantibody). In the schematics shown in FIGS. 7A and 7B, at least one component of the fragment complementation system is an agent that includes the corresponding antigen or epitope for the antibody. For example, in the depicted embodiments, a first agent includes a first peptide fragment and a first targeting domain that binds the antibody directly (e.g., an anti-mAb protein such as protein A, protein G, or protein L), while a second agent includes a second peptide fragment and a second targeting domain (e.g., the antigen or epitope to the targeted antibody). FIG. 7B differs from FIG. 7A in that, for FIG. 7A, the anti-mAb protein is fused or conjugated to β9 and the antigen is fused or conjugated to β10*. Conversely, in FIG. 7B, the anti-mAb protein is fused or conjugated to β10* while the antigen is fused or conjugated to β9. As shown in FIGS. 7A and 7B, binding of (1) the first targeting domain and the second targeting domain to the antibody and (2) the first peptide fragment and the second peptide fragment to a third fragment of the split reporter protein results in an active reporter protein complex, thereby allowing for detection of the antibody.

### Examples

### Recombinant Production of Split-Enzyme Reporter System Components

A tripartite split enzyme reporter system was engineered starting from NanoBiT®, a commercially available binary split enzyme reporter system for use in identifying protein-protein interactions. NanoBiT® includes an 11-amino acid peptide fragment (1.3 kDa) referred to as 114 and a 159-amino acid peptide fragment (18 kDa) referred to as 11S. The two fragments, when bound to one another, form a 10-stranded beta-barrel protein, with the 11S peptide fragment corresponding to the first nine beta stands of the protein, and 114 corresponding to the tenth beta strand.

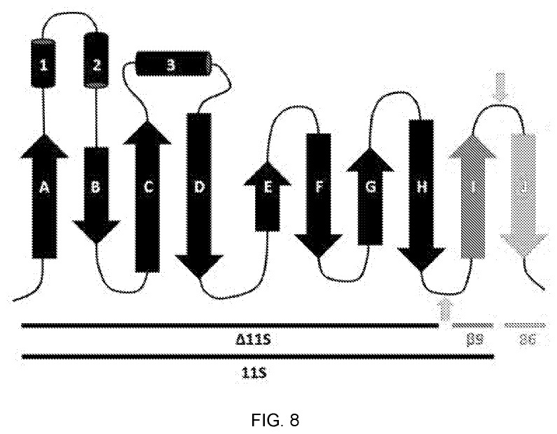

To develop the tripartite split enzyme, the 11S fragment was effectively split into two components: an 11-amino acid peptide (β9) corresponding to the most C-terminal amino acids of 11S, and a 148-amino acid peptide (A11S, 16.5 kDa) corresponding to the most N-terminal amino acids of 11S. Further, instead of using 114 (a relatively low affinity peptide, apparent K_{D} of 190 M) for the remaining β10 peptide fragment, a peptide “β10*” (VSGWRLFKKIS) with higher affinity (apparent K_{D} of ˜700 nM) for the remaining portions of the complex was used. In short, the tripartite enzyme reporter system included (1) a relatively large N-terminal fragment (Δ11S) that includes the first eight beta strands of the intact complex, (2) a relatively short peptide fragment (β9) that corresponds with the ninth beta strand, and (3) another relatively short peptide fragment (β10*) that corresponds with the tenth beta strand. Topological representations of the peptide fragments are depicted in FIG. 8. (β10* is sometimes referred to as 86 in the drawings.)

To generate the fragments of the tripartite split enzyme system, Δ11S was produced recombinantly, while β9 and β10* were synthesized by solid-phase peptide synthesis.

### Enzymatic Activity and Kinetics of Split Enzyme System

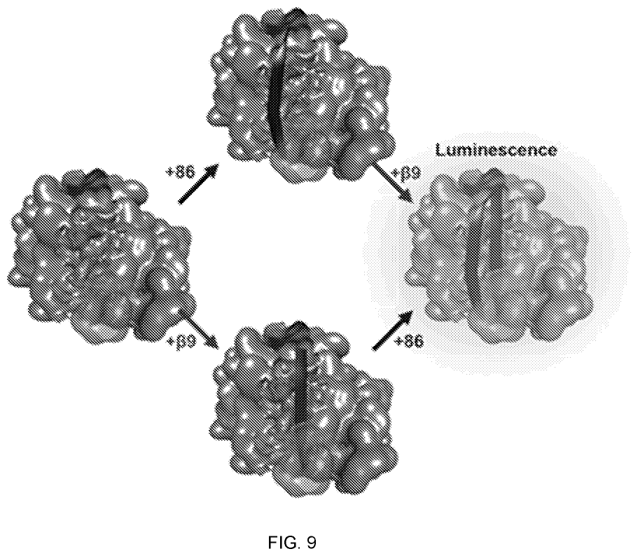

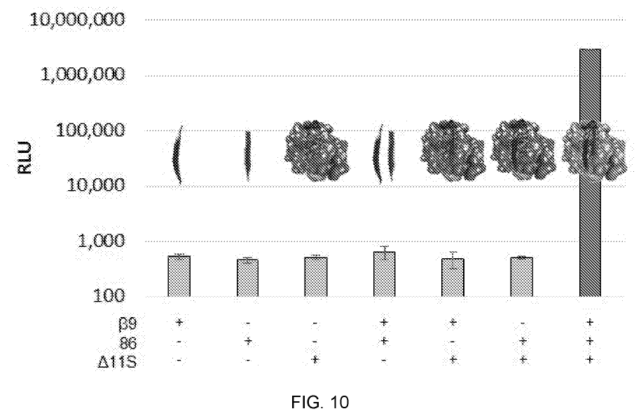

To confirm that the components of the reporter system were active only when all three peptide fragments were present as envisioned in the structural models shown in FIG. 9, the activity of various combinations of the peptide fragments (each at 1 μM concentration) was investigated in the presence of a substrate (furimazine). As shown in FIG. 10, the mixture of all three components is the only combination that produced significant luminescence. These findings confirmed that these fragments can be used to create a luminescent complementation system.

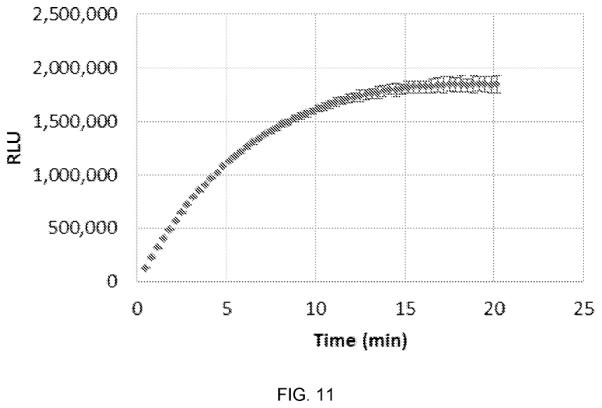

Further, the luminescent signal was monitored after the addition of Δ11S (1 μM) into a solution that included both β9 and β10* (each at 1 μM). The results, as depicted in FIG. 11, show that, under the reaction conditions, 90% of the maximal signal was obtained within approximately 10 minutes, demonstrating that complex formation can be rapid.

### Molecular Modeling and Screening to Identify Antibody Pairs for Target-Engaged Complementation in the Presence of HER2

Epidermal growth factor receptor 2 (HER2 or ErbB-2) was chosen for development and validation of a target-engaged complementation system. HER2 is a highly characterized biomarker (Kurebayashi, J., Breast Cancer 8, 45-51 (2001); Ross, J. S. et al. Mol Cell Proteomics 3, 379-98 (2004); Ross, J. S. et al., Oncologist 8, 307-25 (2003); Yu & Hung, Oncogene 19, 6115-21 (2000)) that has been the focus of significant research efforts stemming from its role in oncogenesis, including breast (Menard et al., J Cell Physiol 182, 150-62 (2000)), ovarian (Teplinsky & Muggia, Gynecol Oncol 135, 364-70 (2014); McAlpine et al., BMC Cancer 9, 433 (2009)), uterine (Santin et al., Clin Cancer Res 8, 1271-79 (2002); Todeschini et al., Br J Cancer 105, 1176-82 (2011)), gastric (Jorgensen, J. T., World J Gastroenterol 20, 4526-35 (2014); Hechtman & Polydorides, Arch Pathol Lab Med 136, 691-97 (2012)), and lung (Heinmoller et al., Clin Cancer Res 9, 5238-43 (2003)) cancers. HER2 expression levels can be used to diagnose and differentiate patients (Wesola & Jelen, Adv Clin Exp Med 24, 899-903 (2015)). Further, the extracellular domain of HER2 is often shed from cells, and its detection in serum can be used to monitor treatment and recurrence (Di Gioia et al., Clin Chim Acta 430, 86-91 (2014); Witzel et al., Breast Cancer Res Treat 123, 437-445 (2010); Esteva et al., Breast Cancer Res 7, R436-443 (2005)). Quantifying HER2 levels through targeted-engaged complementation can be simple and rapid, and may reduce the potential for error inherent to the multi-step protocols of typical immunoassays.

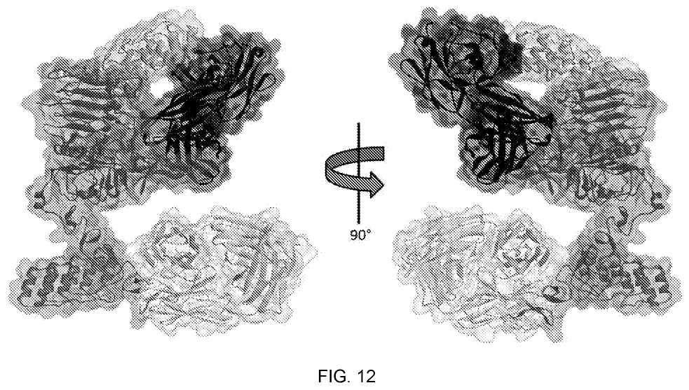

Molecular modeling was used to identify proximal, but non-overlapping epitopes on HER2. More specifically, crystal structures in the Protein Data Bank of antibodies and other binders bound to HER2 were analyzed by superimposing the HER2 portion of the crystal structures (Trastuzumab, PDB ID: 1N8Z; Pertuzumab, PDB ID: 1S78; DARPin G3, PDB ID: 4HRN; and DARPin 9.29, PDB ID: 4HRL (see e.g., Cho et al., Nature 421, 756-60 (2003); Zhou et al., J Biol Chem 286, 31676-83 (2011); Fisher et al., J Mol Biol 402, 217-29 (2010); Eigenbrot et al., PNAS 107, 15039-44 (2010)). The superimposed structures are shown in FIG. 12. Four HER2 binders were selected based primarily on their non-competitive binding of HER2 (see FIG. 12). The selected binders included two antibodies (Trastuzumab and Pertuzumab) (Franklin et al., Cancer Cell 5, 317-28 (2004); Pegram et al., J Clin Oncol 16, 2659-71 (1998); Walshe et al., Clin Breast Cancer 6, 535-39 (2006); Plosker & Keam, Drugs 66, 449-75 (2006); Jost et al., Structure 21, 1979-91 (2013); Owen et al., J Control Release 172, 395-404 (2013); Lewis et al., Nat Biotechnol 32, 191-98 (2014); Epa et al., PLOS One 8, e59163 (2013); Zahnd et al., J Mol Biol 369, 1015-28 (2007); Carter et al., PNAS 89, 4285-89 (1992)) and two designed ankyrin repeated proteins (DARPins, G3 and 9.29). Both Trastuzumab and Pertuzumab are FDA-approved drugs for the treatment of HER2-positive breast cancer. As shown in FIG. 12, each of these binders binds to separate regions of the extracellular domain of HER2. While Trastuzumab binds domain IV close to the cell surface and does not interfere with HER2 oligomerization, Pertuzumab binds the protruding knob of domain II that is necessary for dimerization. Like Trastuzumab, DARPin G3 also binds domain IV, but binds the opposite side from the Trastuzumab binding site. DARPin 9.29 binds domain I adjacent to Pertuzumab, suggesting that these two binders would be suitable binding domains for a target-engaged complementation pair. All four of these proteins bind HER2 with high affinity (K_{D} values between 0.09 nM and 3 nM), and do so on four distinct epitopes, suggesting that any two of them could yield a functional target-engaged complementation pair. As such, these binders were selected for screening. In addition to these binders that bind to characterized epitopes, an additional antibody that has not been crystallized but is known to not compete with Trastuzumab for binding—73J—was also screened.

To screen for functional target-engaged complementation pairs, constructs were designed in which each peptide fragment is fused to all five binders (either on the N- or C-terminus). For the antibodies, a fragment antigen binding fragment (Fab) was used, providing the opportunity to make a fusion at either of the light or heavy chain termini. Selecting one of each of the N- and C-termini produced a set of fusion protein fragments for screening (10 fusions with (9×10 fusions with β10* for a total of 100 possible pairs). For each of these fusions, a linker of 18 amino acids (SEQ ID NO 4) was used between the binder and the peptide. If fully extended, the linker is capable of spanning ˜70 angstroms, suggesting that the termini of each binding pair should be within ˜140 angstroms. As determined by molecular modeling, and shown in Table 1 (below), all pairs of termini are within this distance. (The measured distances ranged from 43-141 angstroms.)

TABLE 1
Distance between potential binding pairs (angstroms)

| | β9-LPert | PertH-β9 | β9-HTras | TrasH-β9 | β9-L73J | 73JH-β9 | β9-9.29 | 9.29-β9 | β9-G3 | G3-β9 |
|---|---|---|---|---|---|---|---|---|---|---|
| β10*-LPert | | | 47 | 43 | | | 67 | 56 | 97 | 66 |
| PertH-β10* | | | 92 | 93 | | | 69 | 52 | 141 | 121 |
| β10*-HTras | 47 | 92 | | | | | 93 | 100 | 47 | 55 |
| TrasH-β10* | 43 | 93 | | | | | 108 | 97 | 111 | 73 |
| β10*-L73J | | | | | | | | | | |
| 73JH-β10* | | | | | | | | | | |
| β10*-9.29 | 67 | 69 | 93 | 108 | | | | | 102 | 87 |
| 9.29-β10* | 56 | 52 | 100 | 97 | | | | | 132 | 104 |
| β10*-G3 | 97 | 141 | 47 | 111 | | | 102 | 132 | | |
| G3-β10* | 66 | 121 | 55 | 73 | | | 87 | 104 | | |

To identify functional target-engaged complementation pairs, all 20 fusions were expressed in E. coli. The SEQ ID numbers corresponding to nucleotide sequences and amino acid sequences for the fusion proteins are set forth in Table 2.

TABLE 2
Sequence Listings

| | |
|---|---|
| β9-L73J | SEQ ID NO 5 (nucleotide) |
| | SEQ ID NO 6 (amino acid: β9-His Tag-light chain) |
| | SEQ ID NO 7 (amino acid: heavy chain) |
| β10*-L73J | SEQ ID NO 8 (nucleotide) |
| | SEQ ID NO 9 (amino acid: β10*-His Tag-light chain) |
| | SEQ ID NO 10 (amino acid: heavy chain) |
| 73JH-β9 | SEQ ID NO 11 (nucleotide) |
| | SEQ ID NO 12 (amino acid: light chain) |
| | SEQ ID NO 13 (amino acid: heavy chain-His Tag-β9) |
| 73JH-β10* | SEQ ID NO 14 (nucleotide) |
| | SEQ ID NO 15 (amino acid: light chain) |
| | SEQ ID NO 16 (amino acid: heavy chain-His Tag-β9) |
| β9-HTras | SEQ ID NO 17 (nucleotide) |
| | SEQ ID NO 18 (amino acid: light chain) |
| | SEQ ID NO 19 (amino acid: β9-His Tag-heavy chain) |
| β10*-HTras | SEQ ID NO 20 (nucleotide) |
| | SEQ ID NO 21 (amino acid: light chain) |
| | SEQ ID NO 22 (amino acid: β10*-His Tag-heavy chain) |
| TrasH-β9 | SEQ ID NO 23 (nucleotide) |
| | SEQ ID NO 24 (amino acid: light chain) |
| | SEQ ID NO 25 (amino acid: heavy chain-His Tag-β9) |
| TrasH-β10* | SEQ ID NO 26 (nucleotide) |
| | SEQ ID NO 27 (amino acid: light chain) |
| | SEQ ID NO 28 (amino acid: heavy chain-His Tag-β10*) |
| β9-LPert | SEQ ID NO 29 (nucleotide) |
| | SEQ ID NO 30 (amino acid: β9-His Tag-light chain) |
| | SEQ ID NO 31 (amino acid: heavy chain) |
| β10*-LPert | SEQ ID NO 32 (nucleotide) |
| | SEQ ID NO 33 (amino acid: β10*-His Tag-light chain) |
| | SEQ ID NO 34 (amino acid: heavy chain) |
| PertH-β9 | SEQ ID NO 35 (nucleotide) |
| | SEQ ID NO 36 (amino acid: light chain) |
| | SEQ ID NO 37 (amino acid: heavy chain- His Tag-β9) |
| PertH-β10* | SEQ ID NO 38 (nucleotide) |
| | SEQ ID NO 39 (amino acid: light chain) |
| | SEQ ID NO 40 (amino acid: heavy chain-His Tag-β10*) |
| β9-G3 | SEQ ID NO 41 (nucleotide) |
| | SEQ ID NO 42 (amino acid: β9-His Tag-G3) |
| β10*-G3 | SEQ ID NO 43 (nucleotide) |
| | SEQ ID NO 44 (amino acid: β10*-His Tag-G3) |
| G3-β9 | SEQ ID NO 45 (nucleotide) |
| | SEQ ID NO 46 (amino acid: His Tag-G3-β9) |
| G3-β10* | SEQ ID NO 47 (nucleotide) |
| | SEQ ID NO 48 (amino acid: His Tag-G3-β10*) |
| β9-9.29 | SEQ ID NO 49 (nucleotide) |
| | SEQ ID NO 50 (amino acid: β9-His Tag-9.29) |
| β10*-9.29 | SEQ ID NO 51 (nucleotide) |
| | SEQ ID NO 52 (amino acid: β10*-His Tag-9.29) |
| 9.29-β9 | SEQ ID NO 53 (nucleotide) |
| | SEQ ID NO 54 (amino acid: 9.29-His Tag-β9) |
| 9.29-β10* | SEQ ID NO 55 (nucleotide) |
| | SEQ ID NO 56 (amino acid: 9.29-His Tag-β10*) |

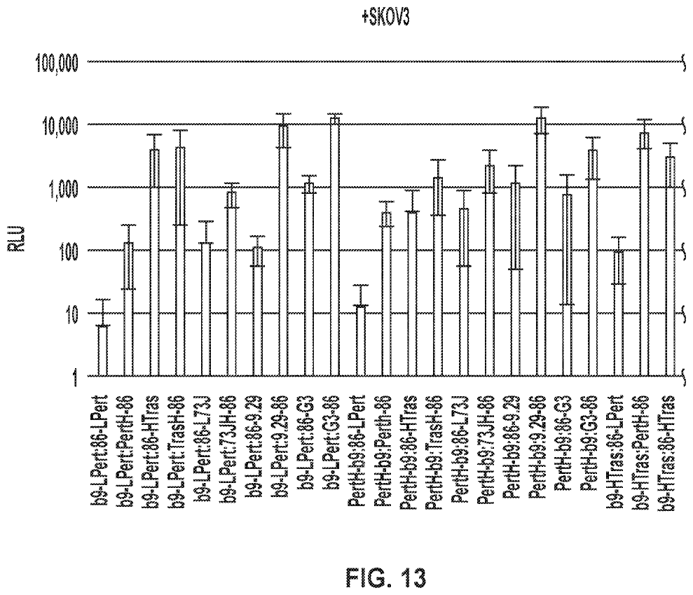

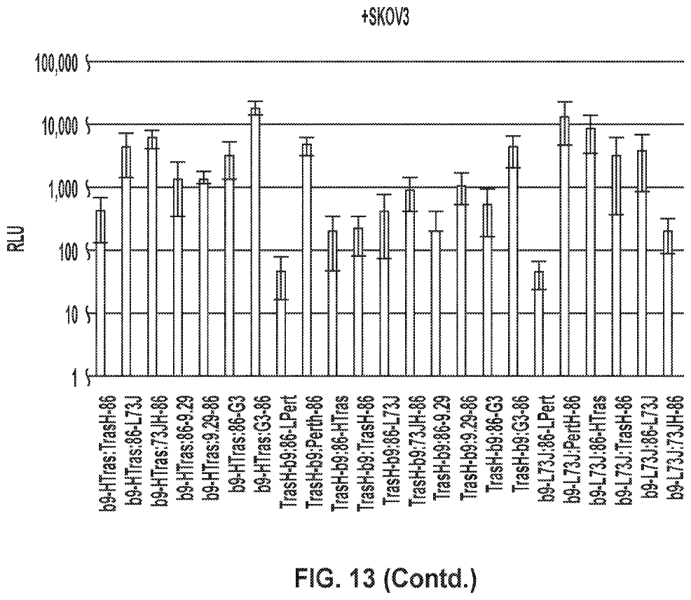

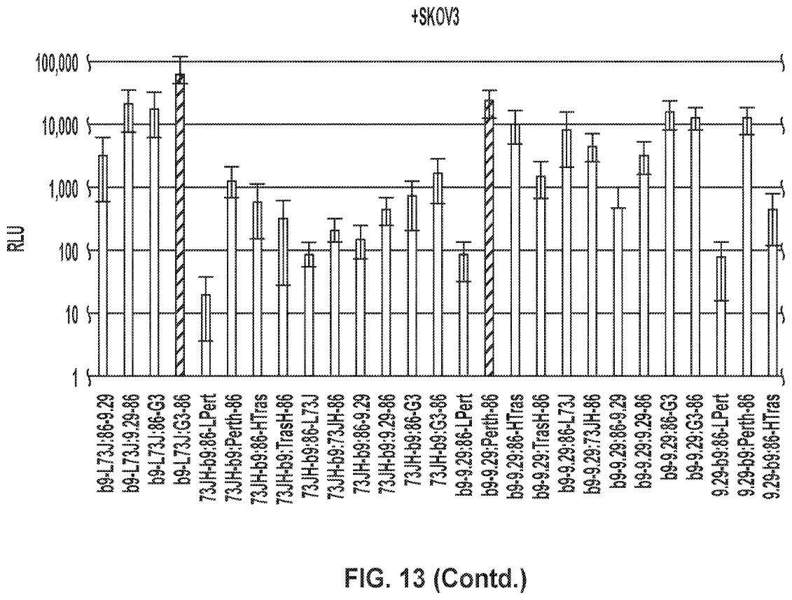

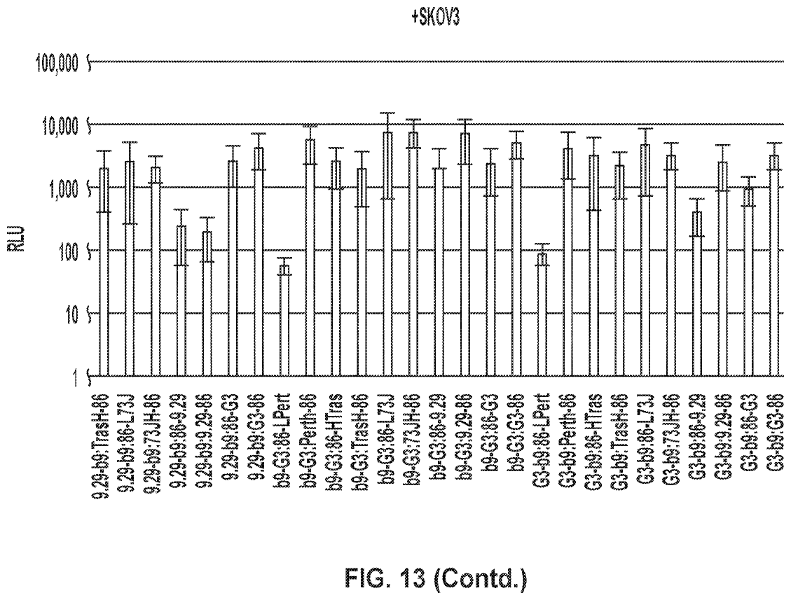

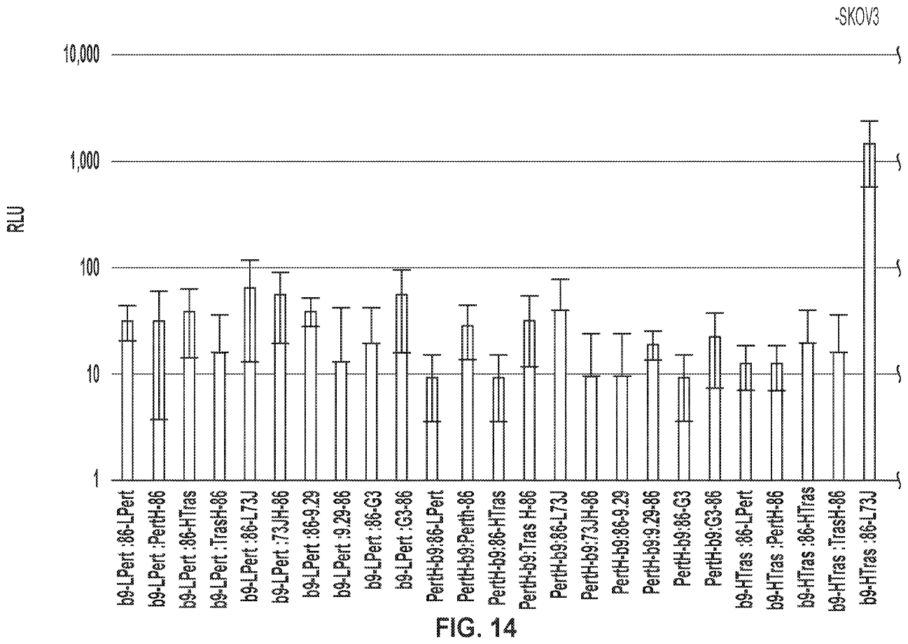

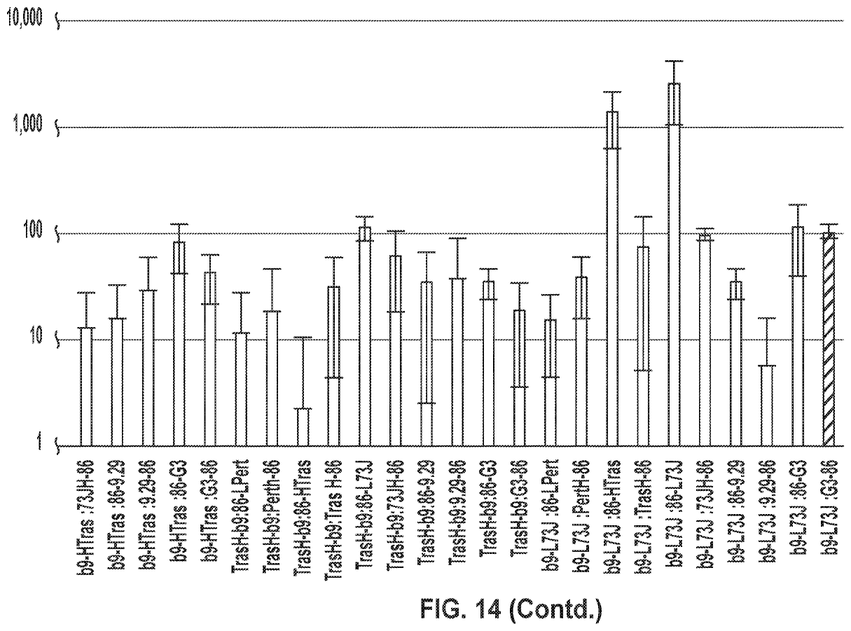

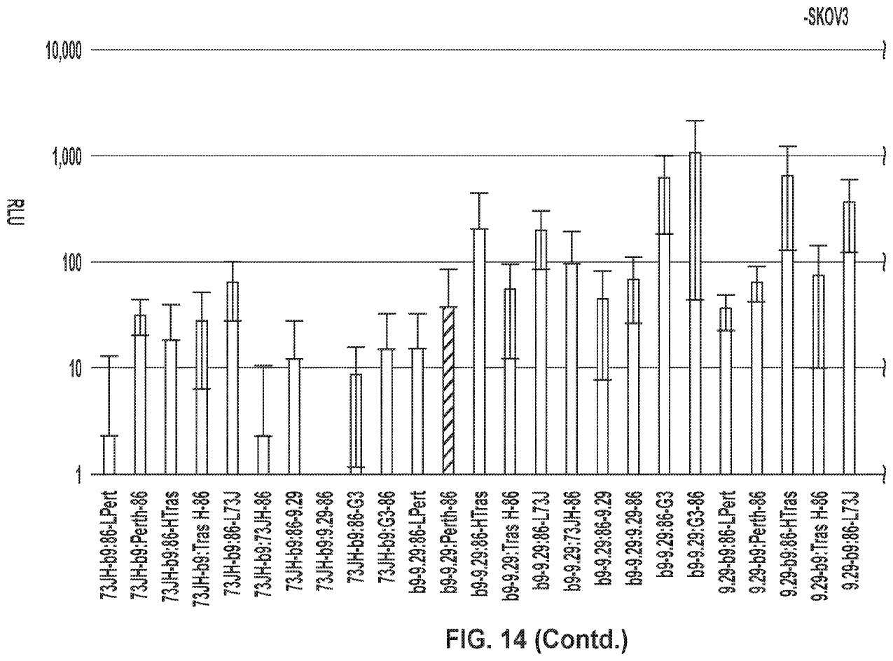

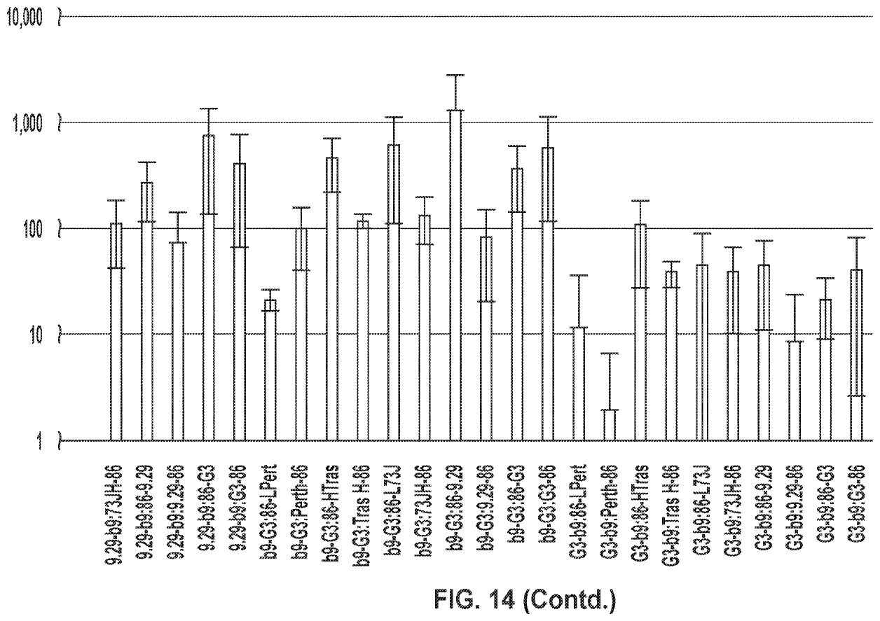

The cells were then lysed, and the concentration was normalized through luminescence. All possible pairs were screened on HER2⁺ SKOV3 cells. More particularly, cells were incubated with the two binder fusion proteins at ambient temperature. The cells were then washed, β11S and substrate (furimazine) were added, and luminescence was measured. The results are shown in FIGS. 13-14, with the −SKOV3 cells of FIG. 14 serving as a control.

The average signal-to-background results from this screen (as performed on three separate days) are summarized in Table 3.

TABLE 3
Signal-to-Background Measurements (Ratio of Relative Luminescence Units)

| | β9-LPert | PerH-β9 | β9-HTras | TrasH-β9 | β9-L73J | 73JH-β9 | β9-9.29 | 9.29-β9 | β9-G3 | G3-β9 |
|---|---|---|---|---|---|---|---|---|---|---|
| β10*-LPert | 0 ± 0 | 0 ± 0 | 3 ± 2 | 1 ± 1 | 1 ± 1 | 1 ± 1 | 2 ± 1 | 2 ± 2 | 2 ± 0 | 3 ± 1 |
| PertH-β10* | 4 ± 3 | 11 ± 4 | 211 ± 81 | 128 ± 31 | 367 ± 221 | 38 ± 17 | 671 ± 284 | 345 ± 130 | 65 ± 29 | 122 ± 80 |
| β10*-HTras | 108 ± 78 | 11 ± 12 | 80 ± 49 | 6 ± 4 | 7 ± 2 | 18 ± 13 | 167 ± 153 | 2 ± 1 | 5 ± 1 | 45 ± 26 |
| TrasH-β10* | 114 ± 104 | 40 ± 29 | 11 ± 7 | 6 ± 3 | 83 ± 71 | 9 ± 8 | 44 ± 22 | 60 ± 45 | 33 ± 26 | 61 ± 39 |
| β10*-L73J | 4 ± 4 | 12 ± 11 | 3 ± 1 | 6 ± 3 | 2 ± 1 | 3 ± 1 | 173 ± 208 | 9 ± 4 | 21 ± 23 | 126 ± 102 |
| 73JH-β10* | 23 ± 9 | 60 ± 37 | 170 ± 43 | 25 ± 12 | 6 ± 3 | 7 ± 2 | 81 ± 52 | 39 ± 26 | 219 ± 91 | 95 ± 35 |
| β10*-9.29 | 3 ± 1 | 31 ± 28 | 38 ± 26 | 5 ± 6 | 93 ± 72 | 4 ± 2 | 13 ± 13 | 1 ± 1 | 4 ± 4 | 12 ± 6 |
| 9.29-β10* | 265 ± 137 | 346 ± 135 | 40 ± 4 | 29 ± 14 | 597 ± 352 | 13 ± 5 | 99 ± 49 | 5 ± 3 | 122 ± 84 | 75 ± 48 |
| 8β10*G3 | 32 ± 8 | 21 ± 20 | 87 ± 45 | 15 ± 10 | 197 ± 80 | 21 ± 14 | 36 ± 13 | 11 ± 12 | 7 ± 3 | 28 ± 12 |
| G3-β10* | 353 ± 63 | 103 ± 62 | 508 ± 108 | 118 ± 58 | 1358 ± 455 | 47 ± 29 | 19 ± 12 | 31 ± 27 | 12 ± 7 | 93 ± 37 |

Within this set, each pair was tested with both possible orientations of the peptides (e.g., G3-β10*: TrasH-39 and G3-β9:TrasH-β10*). There was little correlation between the results obtained upon swapping the peptide fused to each binder. Furthermore, only four of the top 20 pairs involved 39 as a C-terminal fusion. Similarly, four (but not the same four found with C-terminal 39) out of the top 20 pairs involved β10* as an N-terminal fusion. Together, the data suggest that the complementation system functions preferentially, but not exclusively, when 9 is an N-terminal fusion and β10* is a C-terminal fusion. Twenty-three pairs produced a signal (relative luminescence units) at least 100-fold higher than background. The top two pairs—39-L73J:G3-β10* and 39-9.29: PertH-β10*—were selected for further characterization and analysis.

### Validation of Target-Engaged Complementation

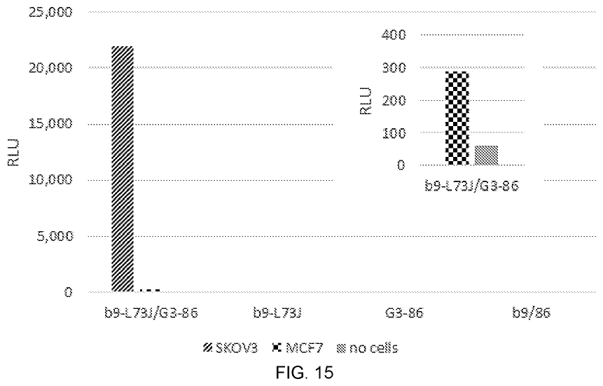

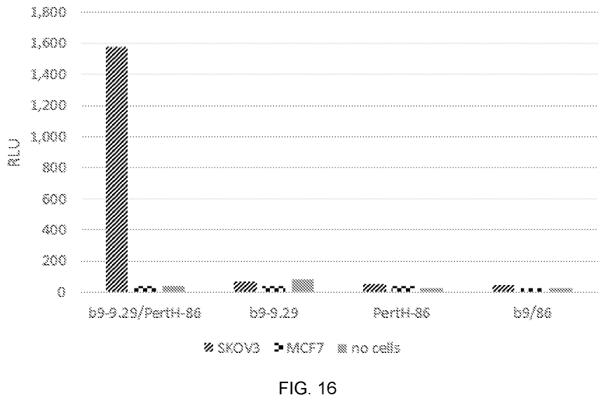

Unpurified antibody and DARPin fusions (β9-L73J:G3-β10* and β9-9.29: PertH-β10*) from lysates were validated by their binding to HER2⁺ SKOV3 cells as described above. As shown in FIGS. 15-16, in the absence of HER2 (no cells), negligible signal is generated. Similarly, no significant luminescence was produced when less than all of the components of the fragment complementation system were added. However, when the proper pair of fusion proteins was introduced into the system, a luminescent signal was produced. The assay also distinguished between low (MCF-7) and high (SKOV3) HER2 expressing cells. See FIGS. 15-16.

Subsequently, the same assay was run with purified fusion proteins. More particularly, the 39-L73J:G3-β10* and 39-9.29:PertH-β10* fusion proteins were produced recombinantly in E. coli. Each of these fusion proteins included a His-Tag, enabling purification using standard immobilized metal affinity chromatography (IMAC) procedures. Block et al., 463 Methods Enzymol 439-73 (2009). The purified proteins retained ≥90% of the activity of the originating lysate, even after storage for over one month.

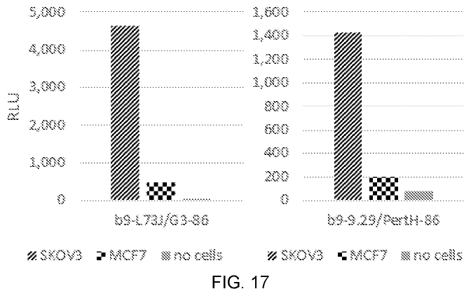

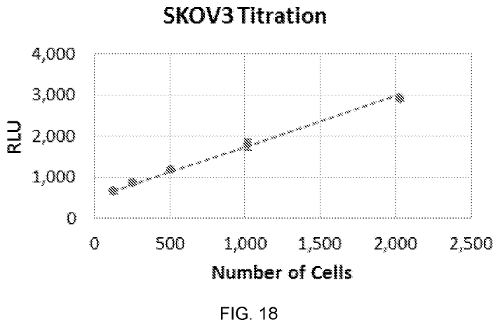

The data for the purified fusion proteins is shown in FIGS. 17 and 18. As shown in these figures, the purified proteins could be used to detect and quantify HER2, and could be used to differentiate between low (MCF-7) and high (SKOV3) HER2-expressing cells.

Immunoassay without Wash Step

HER2⁺ SKOV3 cells were introduced into wells of a multi-well plate. β9-binder, β10*-binder, Δ11S, and furimazine were added to each well. In contrast to other HER2 assays described herein, the unbound antibodies were not washed away. In other words, luminescence was detected without any intervening wash step.

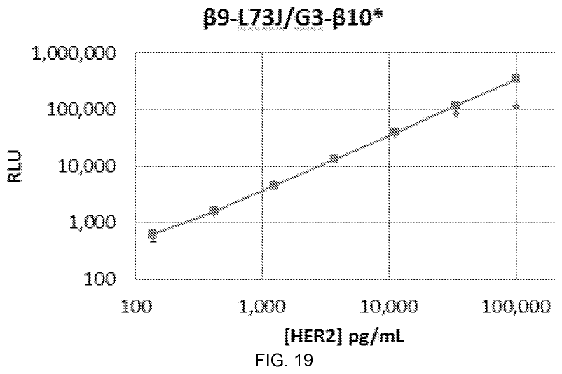

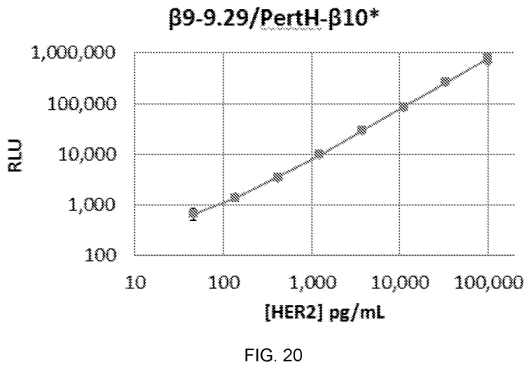

The concentration of each fusion protein was tailored so as to provide sufficient binder to bind the antigen (i.e., HER2) while keeping the concentration of the peptide relatively low so as to minimize complementation (and background signal) in the absence of the antigen. To this end, the concentration of each fusion in both target-engaged complementation pairs was optimized. Low nM concentrations were found to be appropriate. The resulting luminescence data collected two hours after incubation is shown in FIGS. 19-20. More particularly, FIG. 19 provides data for β9-L73J/G3-β10* target-engaged complementation. And FIG. 20 provides data for 39-9.29/PertH-β10* target-engaged complementation.

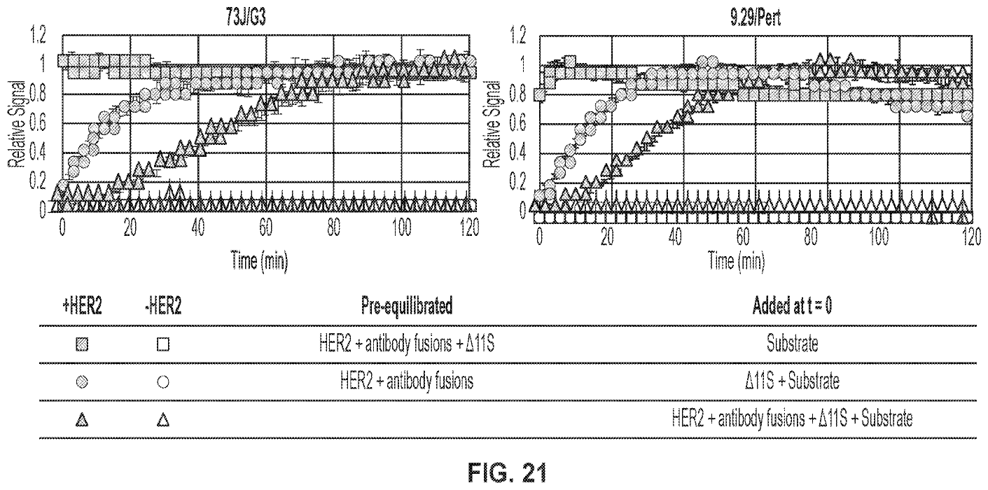

In assays such as this one, where each of the three components of the tripartite split enzyme are added at once, the binding kinetics may influence the optimal time for measuring luminescence. In addition to binding of the targeting domain (e.g., Fab or DARPin) to the analyte (e.g., HER2), Δ11S also needs to bind to B9/β10* to form the active luciferase complex. To evaluate when to measure the signal and to differentiate binding of the targeting domain from formation of the luciferase complex, kinetics studies were performed. As shown in FIG. 21, the signal kinetics from an experiment where each component of the target-engaged complementation system (and substrate) is simultaneously added to the mixture (HER2+β9-binder+β10*-binder+Δ11S+substrate; solid triangles) were compared to the kinetics where (1) both targeting domains (but not Δ11S or substrate) were pre-equilibrated with HER2 (HER2+β9-binder+β10*-binder) (closed circles) and (2) both targeting domains and Δ11S (but not substrate) were pre-equilibrated with HER2 (HER2+β9-binder+β10*-binder+Δ11S) (solid squares). When all components are pre-equilibrated with HER2 (solid squares), luminescence is immediately obtained. If Δ11S is not pre-equilibrated, but added with substrate to pre-equilibrated targeting domains (solid circles and solid triangles), the luminescent signal increases with time according to Δ11S binding and/or the formation of an active luciferase complex. The formation of the luciferase complex is slower for the 73J/G3 combination, reaching approximately 90% of the maximal signal at ˜50 minutes, as compared to ˜30 minutes for 9.29/Pertuzumab. Among other possibilities, this difference in rate may be a reflection of a larger distance of separation between the fused termini. As expected, when the targeting domains are not pre-equilibrated with HER2, an increased time was required to achieve the maximal signal, consistent with the requirement for both targeting domain and Δ11S binding. The kinetics data demonstrate that the maximal signal is obtained within two hours. Furthermore, for 9.29/Pertuzumab, signal-to-background of five was obtained within 20 minutes, demonstrating the feasibility of this relatively fast and simple single-step assay.

To determine the sensitivity of the assay, the following definitions were noted as outlined by Armbruster and Pry (Armbruster & Pry, Clin Biochem Rev 29 Suppl 1, S49-52 (2008)):

$$Limit of Blank ( LoB ) = mean {RLU}_{blank} + 1.645 \times {SD}_{blank} \\ Limit of Detection ( LoD ) = LoB + 1.645 \times {SD}_{low concentration sample}$$

As opposed to using the definition for the limit of detection and extrapolating to a concentration that had not actually been tested, the LoD was defined as the lowest concentration measured that produced a signal greater than the LoB+1.645 times the standard deviation. For 73J/G3, the LoD was 137 pg/mL and the linear range extended up to a concentration of 11 ng/ml. The combination of 9.29 and Pertuzumab was found to exceed these capabilities in terms of both the limit of detection and the linear range. The LoD for this pair was determined to be 45 pg/mL. Although 100 ng/ml seems to be close to lying within the linear range, the small standard deviation of this assay would suggest that it does not, and the highest concentration assayed that remained in the linear range was 33 ng/mL. A previous demonstration using complementation to detect HER2 reported the ability to detect 500 pM HER2 (Stains et al. ACS Chem Biol 5, 943-52 (2010)). These results demonstrate the ability to detect sub-picomolar concentrations (0.7 pM), an improvement of approximately 500-fold. Further, commercially available ELISA kits report sensitivities in the range of 8-24 pg/mL with linear ranges up to approximately 5 ng/ml. The sensitivity and dynamic range demonstrated here is comparable to the sensitivity and dynamic range of these commercially available ELISA assays. This is particularly impressive, considering that many standard ELISA kits involve signal amplification by binding multiple antibodies per antigen and/or multiple enzymes per antibody. In contrast, for every pair of bound targeting domains, the signal demonstrated here is limited to the signal generated from a single active luciferase complex that is formed via target-engaged complementation.

### Analyte Detection in Human Serum

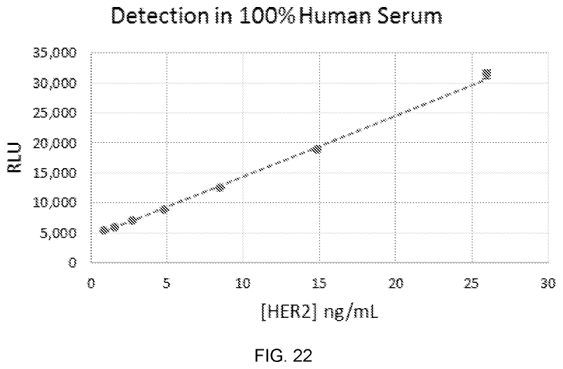

As soluble HER2 may be used to monitor the treatment efficacy and recurrence of cancer (see, e.g., Tse et al., Cancer Treat Rev 38, 133-42 (2012); Mokuyasu et al., Rinsho Byori 60, 612-20 (2012); Cook et al., Anticancer Res 21, 1465-70 (2001)), a target-engaged complementation reporter was used to investigate detection of HER2 in human serum. As the threshold for elevated HER2 in serum has been established at >15 ng/ml, HER2 was spiked into human serum at low ng/ml concentrations. A target-engaged complementation assay was then performed using the combination of 9.29 and Pertuzumab (for 39-9.29/PertH-β10*). Given the complex nature and high concentration of proteins in serum, serum is a particularly challenging mixture for a solution-based application of target-engaged complementation. Despite these challenges, target-engaged complementation successfully differentiated between small differences in HER2 concentrations and was linear across the low ng/ml range as shown in FIG. 22. This demonstrates that target-engaged complementation can be used to differentiate between biologically relevant concentrations of HER2 in human serum.

The foregoing data show that HER2 can be quantified via a simple, rapid, and sensitive method. The method is generalizable to other analytes (e.g., proteins) of interest. Further, while the data disclosed herein is most analogous to a traditional ELISA, analogous methods may be used in immunocytochemistry and immunohistochemistry as well.

Target-Engaged Complementation with Split Enzyme Fragments that are Produced by Chemical Conjugation

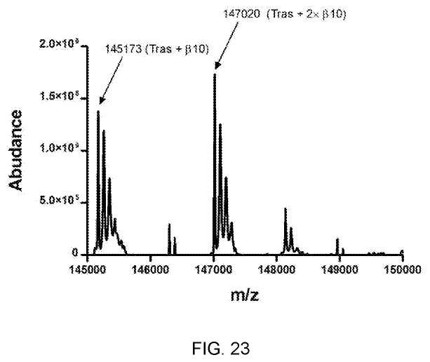

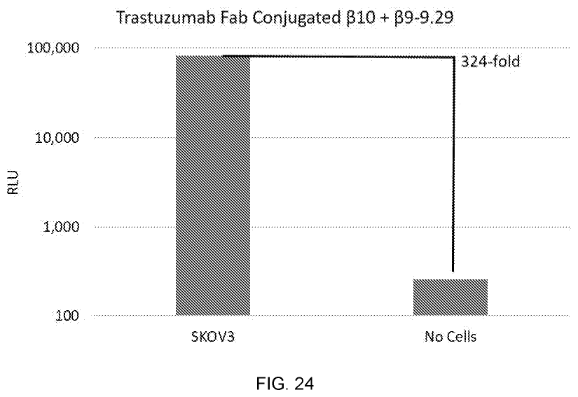

A maleimide-β10* peptide was chemically conjugated to the full IgG form of Trastuzumab using 2-iminothiolane. Conjugation was verified by mass spectrometry as shown in FIG. 23. The mass spectrometry data showed that either one or two β10* fragments were conjugated per antibody. The utility of the chemically conjugated fragment for use in target-engaged complementation was then investigated by combining the chemically conjugated fragment with complementary peptide fragments (β9-9.29 and Δ11S) in the presence and the absence of HER2⁺ cells (SKOV3). The results are shown in FIG. 24. A 324-fold increase in signal was observed in the presence of HER2⁺ cells, demonstrating that a chemically conjugated peptide fragment can be used for target-engaged complementation.

### Target-Engaged Complementation Using Protein A and Protein G as Targeting Domains

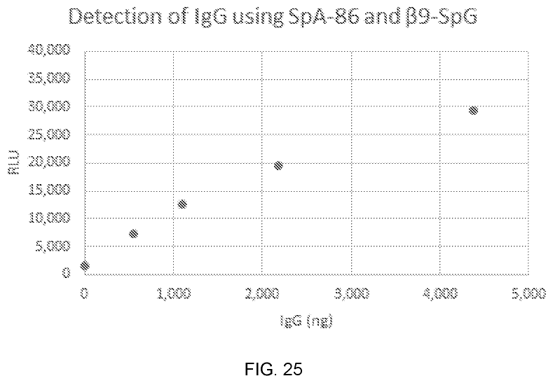

A split reporter system was used to detect IgG in solution. More specifically, a Protein A-β10* fusion protein and a Protein G-89 fusion protein were produced recombinantly. The ability of these peptide fragments to detect IgG (based on the affinity of Protein A and Protein G for IgG) was evaluated by target-engaged complementation. More specifically, the two fusion protein fragments were combined with Δ11S in the presence of various amounts of IgG, and luminescence was measured. The resulting data is shown in FIG. 25. The data show that increased luminescent signal is generated with increasing concentrations of IgG, and that this target-engaged complementation system can be used to assess IgG concentrations in solution.

### Further Target-Engaged Complementation Studies

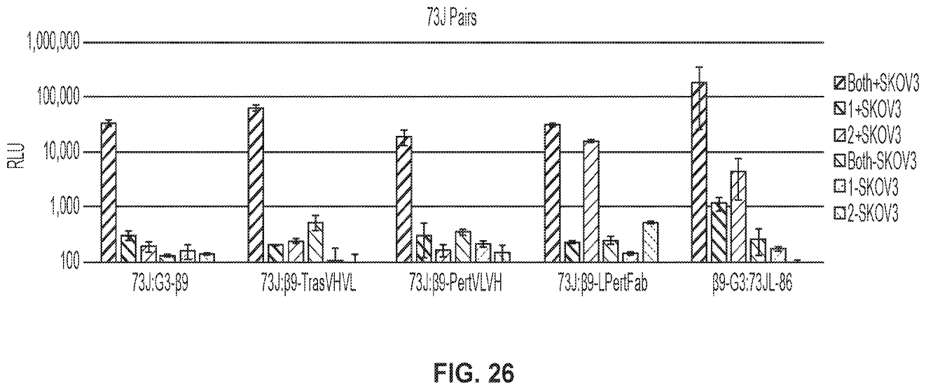

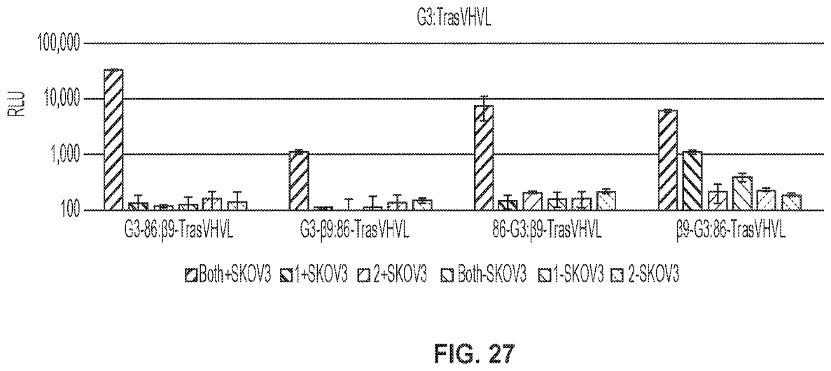

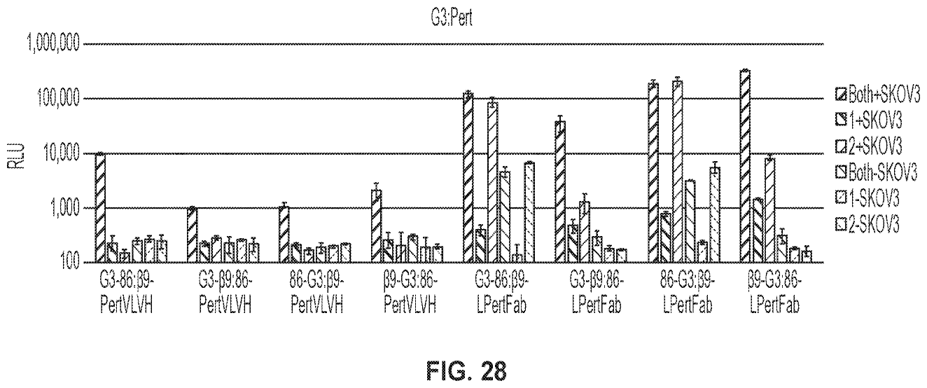

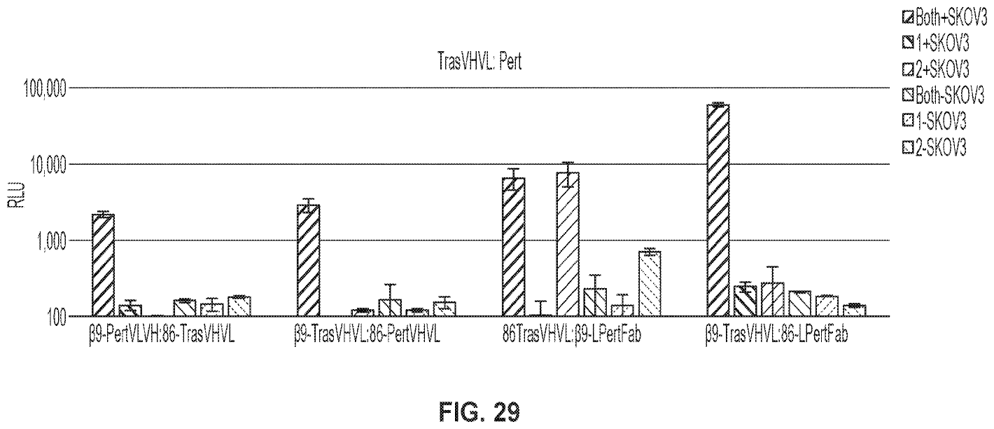

The various fusion proteins shown in FIGS. 26-29 were recombinantly produced. More particularly, 73J and PertFab domains were produced as Fabs, G3 was produced as a DARPin, and TrasVHVL and PertVLVH were produced as single-chain variable fragments (scFv). Each fragment of each split enzyme reporter system was combined with various other fragments of the split enzyme reporter system. For example, as shown in the first set of bar graphs of FIG. 26 for 73J-β10*: G3-β9, each of the following combinations of elements was combined with Δ11S and substrate in either the presence or the absence of HER2⁺ cells: (1) 73J-β10* and G3-β9, (2) 73J-β10* alone, and (3) G3-β9 alone. Similar tests were carried out for twenty other protein complexes. The results shown in FIGS. 26-29 show that a wide variety of different targeting domains may be used for target-engaged complementation.

### Detection of Adalimumab

Embodiments of the protein-fragment complementation system described herein (see FIG. 7 for a representative depiction) were used to detect adalimumab (also known as Humira), which is a monoclonal antibody with affinity to an epitope of tumor necrosis factor (TNF), also known as tumor necrosis factor alpha (TNFα). For instance, fusion proteins were created, where each fusion protein included both (1) a targeting domain (e.g., TNF, protein L, or Protein A) with affinity for adalimumab and (2) a peptide fragment β9 or β10*) of a split reporter protein.

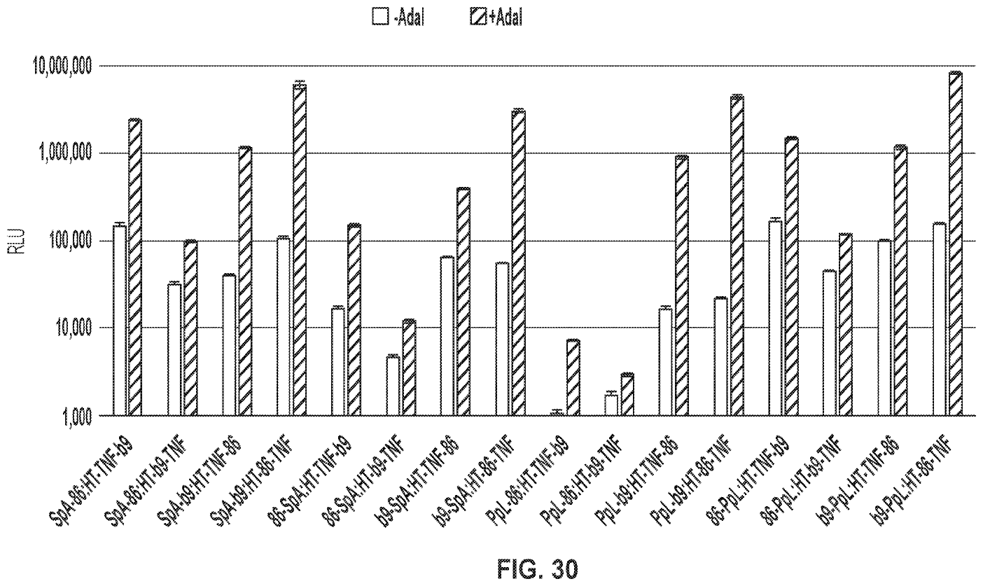

More particularly, Protein A (SpA), Protein L (PpL), and TNF (as a fusion to HaloTag® (“HT”)) were expressed in E. coli (T7 SHuffle Express) as fusions with 39 or β10* at the N- or C-terminus. A lysate was prepared by sonicating the cells and centrifuging to separate out the insoluble material. Each TNF lysate was tested in combination with the opposite peptide (β9 with β10, or β10 with β9) fusion of Protein A and Protein L diluted in PBST. The luminescence produced in the absence (−Adal) or presence (+Adal) of 10 μg/mL adalimumab is shown in the bar graph of FIG. 30. The values shown are the averages from three replicate wells±standard deviation. The sequence listings for some of the fusion proteins (and corresponding nucleotide sequences) are identified in Table 4.

TABLE 4
Sequence Listings

| | |
|---|---|
| HT-β9-TNF | SEQ ID NO 57 (nucleotide) |
| | SEQ ID NO 58 (amino acid) |
| HT-TNF-β9 | SEQ ID NO 59 (nucleotide) |
| | SEQ ID NO 60 (amino acid) |
| HT-β10*-TNF | SEQ ID NO 61 (nucleotide) |
| | SEQ ID NO 62 (amino acid) |
| HT-TNF-β10* | SEQ ID NO 63 (nucleotide) |
| | SEQ ID NO 64 (amino acid) |
| β9-TNF | SEQ ID NO 65 (nucleotide) |
| | SEQ ID NO 66 (amino acid) |
| TNF-β9 | SEQ ID NO 67 (nucleotide) |
| | SEQ ID NO 68 (amino acid) |
| β10*-TNF | SEQ ID NO 69 (nucleotide) |
| | SEQ ID NO 70 (amino acid) |
| TNF-β10* | SEQ ID NO 71 (nucleotide) |
| | SEQ ID NO 72 (amino acid) |
| β9-PpL | SEQ ID NO 73 (nucleotide) |
| | SEQ ID NO 74 (amino acid) |
| PpL-β9 | SEQ ID NO 75 (nucleotide) |
| | SEQ ID NO 76 (amino acid) |
| β10*-PpL | SEQ ID NO 77 (nucleotide) |
| | SEQ ID NO 78 (amino acid) |
| PpL-β10* | SEQ ID NO 79 (nucleotide) |
| | SEQ ID NO 80 (amino acid) |
| β9-SpA | SEQ ID NO 81 (nucleotide) |
| | SEQ ID NO 82 (amino acid) |
| SpA-β9 | SEQ ID NO 83 (nucleotide) |
| | SEQ ID NO 84 (amino acid) |
| β10*-SpA | SEQ ID NO 85 (nucleotide) |
| | SEQ ID NO 86 (amino acid) |
| SpA-β10* | SEQ ID NO 87 (nucleotide) |
| | SEQ ID NO 88 (amino acid) |
| β9-SpGA1 | SEQ ID NO 89 (nucleotide) |
| | SEQ ID NO 90 (amino acid) |
| SpGA1-β9 | SEQ ID NO 91 (nucleotide) |
| | SEQ ID NO 92 (amino acid) |
| β10*-SpGA1 | SEQ ID NO 93 (nucleotide) |
| | SEQ ID NO 94 (amino acid) |
| SpGA1-β10* | SEQ ID NO 95 (nucleotide) |
| | SEQ ID NO 96 (amino acid) |

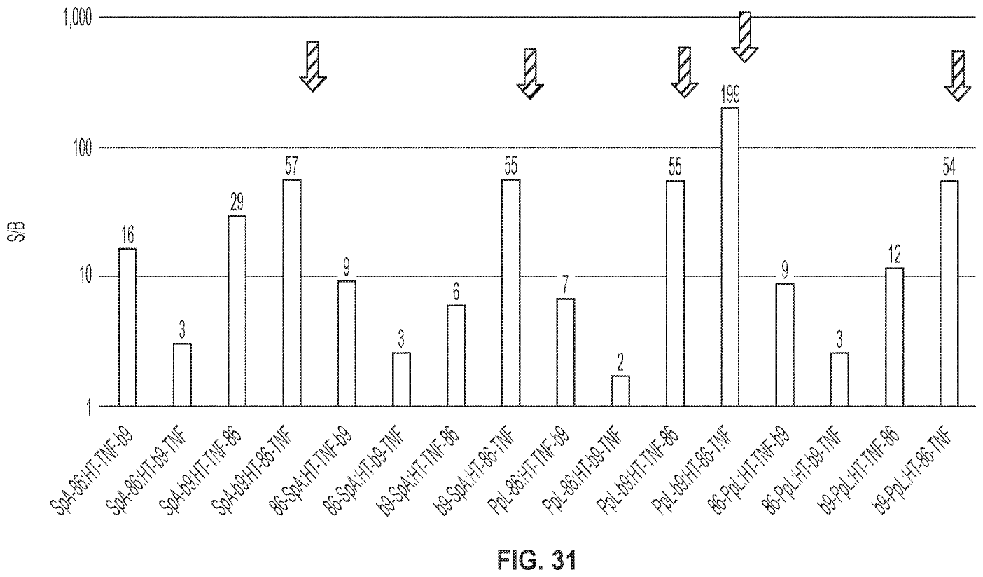

As shown in FIG. 30, increased luminescent signal was observed in the presence of adalimumab, demonstrating that the assay may be used to detect the antibody. The ratio of signal to noise for each pair of fusion proteins is shown in FIG. 31. Signal-to-background values were determined by dividing the luminescence produced in the presence of adalimumab (+Adal) by the luminescence produced in the absence of adalimumab (−Adal).

Four of the pairs of fusion proteins (protein A-β9:HT-β10*-TNF; β9-protein A:HT-β10*-TNF; protein L-β9:HT-TNF-β10*; protein L-β9:HT-β10*-TNF) were selected for further studies based on the high signal-to-noise ratio observed in the experiments reported in FIGS. 30 and 31.

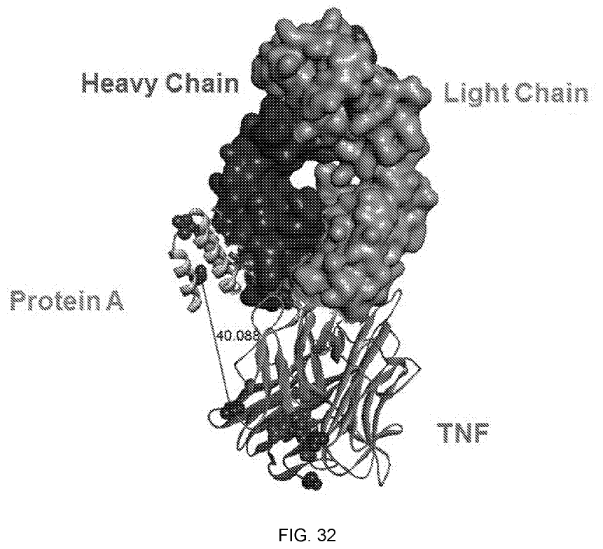

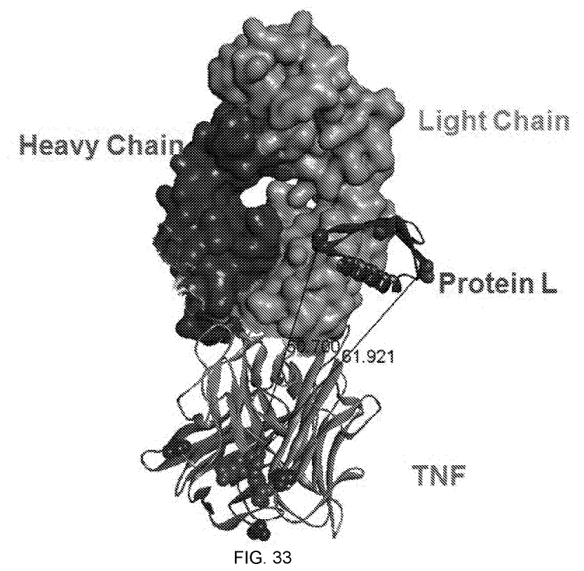

For example, the four pairs of fusion proteins were used to detect adalimumab and infliximab (another antibody with affinity for TNF). Infliximab is shown bound to TNF and protein A in FIG. 32 and bound to TNF and protein L in FIG. 33. More particularly, infliximab was superimposed with trastuzumab using crystal structures in which infliximab is bound to TNF (PDB ID: 4G3Y) and trastuzumab is bound by Protein A and Protein L (PDB ID: 4HKZ). Once superimposed, trastuzumab was removed leaving infliximab, Protein A, Protein L, and TNF in their expected positions within the complex. The distance from the N-terminus of Protein A to the N-terminus of TNF was estimated to be 40 Å as shown in FIG. 32. The distance from the N-terminus and C-terminus of Protein L to the C-terminus of TNF were estimated to be 50.7 Å and 61.9 Å, respectively, as shown in FIG. 33.

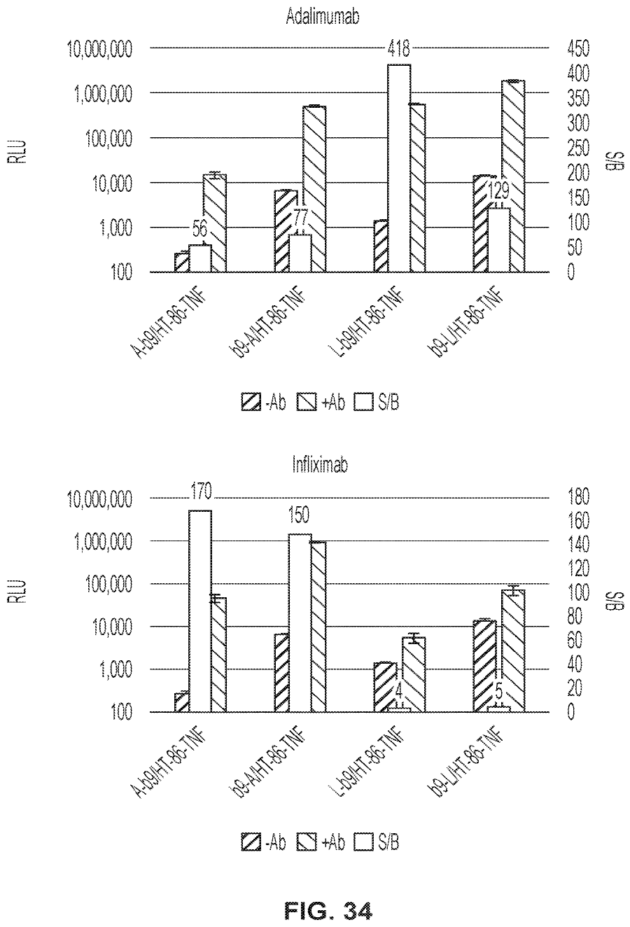

A lysate of HaloTag®-β10*-TNF was tested combination with β9 at the N- and C-terminus of Protein A and Protein L. The luminescence in the absence of antibody (−Ab) or presence of 10 μg/mL antibody (+Ab) is shown (FIG. 34; left y-axis) as well as the signal-to-background ratio (FIG. 34; +Ab signal divided by −Ab signal; right y-axis). Values shown are the averages from three replicate measurement±standard deviation.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method.

Each of the references cited herein is hereby incorporated by reference in its entirety. Further, reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated by one of skill in the art with the benefit of this disclosure that in the above description of embodiments, various features are sometimes grouped together in a single embodiment, figure, or description thereof for the purpose of streamlining the disclosure. This method of disclosure, however, is not to be interpreted as reflecting an intention that any claim requires more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. It will be apparent to those having skill in the art that changes may be made to the details of the above-described embodiments without departing from the underlying principles of the present disclosure.

1.-91. (canceled)

92. A kit comprising for detecting an analyte in a mixture, the kit comprising:
a first agent comprising a first targeting domain for binding to the analyte and a first peptide fragment of a split reporter protein;
a second agent comprising a second targeting domain for binding to the analyte and a second peptide fragment of the split reporter protein;
a third agent comprising a third fragment of the split reporter protein;
wherein the first targeting domain is configured to selectively bind to a first target region of the analyte and the second targeting domain is configured to selectively bind to a second target region of the analyte, and the first and second target regions of the analyte are different from one another; and
wherein the third agent is configured to bind to the first and second agents to form an active reporter complex.

93. The kit of claim 92, wherein the first peptide fragment has a mass of less than 3 kDa.

94. The kit of claim 92, wherein the second peptide fragment has a mass of less than 3 kDa.

95. The kit of claim 92, wherein the first targeting domain and the second targeting domain are each independently selected from the group consisting of an antibody, a designed ankyrin repeat protein, an affibody, a monobody, and an aptamer, or portions thereof.

96. The kit of claim 95, wherein at least one of the first targeting domain and the second targeting domain is an antibody or a portion thereof.

97. The kit of claim 95, wherein at least one of the first targeting domain and the second targeting domain is a fragment antigen binding fragment (Fab) or a single-chain variable fragment.

98. The kit of claim 95, wherein at least one of the first targeting domain and the second targeting domain is a designed ankyrin repeat protein.

99. The kit of claim 92, wherein the analyte is a monomeric protein.

100. The kit of claim 99, wherein the first target region and the second target region are different in structure.

101. The kit of claim 99, wherein the first target region and the second target region are substantially identical in structure, but are located at separate sites on the monomeric protein.

102. The kit of claim 92, wherein the analyte is a multimeric protein complex.

103. The kit of claim 102, wherein the first targeting domain and the second targeting domain bind to adjacent proteins of the multimeric protein complex.

104. The kit of claim 102, wherein the multimeric protein complex is a homomultimeric protein complex.

105. The kit of claim 102, wherein the multimeric protein complex is a heteromultimeric protein complex.

106. The kit of claim 92, wherein the first target region and the second target region are separated by less than 300 angstroms.

107. The kit of claim 92, wherein the split reporter protein is a beta-barrel protein.

108. The kit of claim 92, wherein the split reporter protein is a split enzyme, and wherein the kit further comprises a substrate of the split enzyme.

109. The kit of claim 108, wherein the enzymatic activity of the enzyme on the substrate results in emission of a detectable signal.

110. The kit of claim 109, wherein the split enzyme is a split luciferase, and the detectable signal is luminescence.

111. The kit of claim 108, wherein the split enzyme catalyzes the conversion of furimazine to furimamide.

112. The kit of claim 108, wherein the first peptide fragment is less than or equal to 11 amino acids in length.

113. The kit of claim 108, wherein the second peptide fragment is less than or equal to 11 amino acids in length.

114. The kit of claim 108, wherein the first peptide fragment and the second peptide fragment are each 11 amino acids in length.

115. The kit of claim 108, wherein the third peptide fragment has a mass of between 16 kDa and 17 kDa.

116. The kit of claim 92, wherein the split reporter protein is a split fluorescent protein.

117. The kit of claim 116, wherein the split fluorescent protein is a split green fluorescent protein.

118. The kit of claim 92, wherein one or both of the first agent and the second agent are recombinant fusion proteins.

119. The kit of claim 92, wherein the first agent, the second agent, and the third agent are configured to combine to form a non-covalent complex of the split reporter protein.
